SEQUENCE LISTING (19 sequences)

SEQ ID NO: 1 - RNA, 27 nt, Artificial Sequence
  Derived from Chikungunya virus strain TSI-GSD-218
gagauguuauuuuguuuuuaauauuuc

SEQ ID NO: 2 - RNA, 34 nt, Artificial Sequence
  Derived from Ebola virus Zaire strain
aagaagaaauagauuuauuuuuaaauuuuugugu

SEQ ID NO: 3 - RNA, 33 nt, Artificial Sequence
  Derived from Japanese encephalitis virus
guuuaucugugugaacgauagugcaguuuaaac

SEQ ID NO: 4 - RNA, 22 nt, Artificial Sequence
  Derived from Japanese encephalitis virus
guuuaucugugugaacgauagu

SEQ ID NO: 5 - RNA, 13 nt, Artificial Sequence
  Derived from Japanese encephalitis virus
guuuaucugugug

SEQ ID NO: 6 - RNA, 20 nt, Artificial Sequence
  Derived from Japanese encephalitis virus
cugugugaacgauagugcag

SEQ ID NO: 7 - RNA, 23 nt, Artificial Sequence
  Derived from Japanese encephalitis virus
guuuaucugugugcaguuuaaac

SEQ ID NO: 8 - RNA, 26 nt, Artificial Sequence
  Derived from Murray valley encephalitis virus
uuuuuuggagcuuuugauuucaaaug

SEQ ID NO: 9 - RNA, 20 nt, Artificial Sequence
  Derived from Murray valley encephalitis virus
uuuuuuggagcuuuugauuu

SEQ ID NO: 10 - RNA, 20 nt, Artificial Sequence
  Derived from Murray valley encephalitis virus
uuuggagcuuuugauuucaa

SEQ ID NO: 11 - RNA, 21 nt, Artificial Sequence
  Derived from Murray valley encephalitis virus
uuauuuuguuuuuaauauuuc

SEQ ID NO: 12 - RNA, 20 nt, Artificial Sequence
  Derived from Murray valley encephalitis virus
uuauuuuguuuuuaauauuu

SEQ ID NO: 13 - RNA, 20 nt, Artificial Sequence
  Derived from Murray valley encephalitis virus
uguuauuuuguuuuuaauau

SEQ ID NO: 14 - RNA, 21 nt, Artificial Sequence
  Derived from Murray valley encephalitis virus
agauguuauuguuuaauauuu

SEQ ID NO: 15 - RNA, 18 nt, Artificial Sequence
  Derived from Ebola virus Zaire strain
agaaauagauuuauuuuu

SEQ ID NO: 16 - RNA, 22 nt, Artificial Sequence
  Derived from Ebola virus Zaire strain
acaaaaaagaauaaauuuuugu

SEQ ID NO: 17 - RNA, 22 nt, Artificial Sequence
  Derived from Ebola virus Zaire strain
auuuauuuuuaaauuuuugugu

SEQ ID NO: 18 - RNA, 21 nt, Artificial Sequence
  Derived from Ebola virus Zaire strain
acacaaaaaagauuuuugugu

SEQ ID NO: 19 - RNA, 19 nt, Artificial Sequence
  Derived from Ebola virus Zaire strain
uggacacacaaaaaagaag

(19) United States
(12) Patent Application Publication
(10) Pub. No.: US 2012/0177701 A1
(43) Pub. Date: Jul. 12, 2012

(54) COMPOSITIONS COMPRISING IMMUNOSTIMULATORY NUCLEIC ACIDS AND RELATED METHODS

(75) Inventors: Petr O. Ilyinskii, Cambridge, MA (US); Grayson B. Lipford, Watertown, MA (US)

(73) Assignee: SELECTA BIOSCIENCES, INC., Watertown, MA (US)

(21) Appl. No.: 13/341,325

(22) Filed: Dec. 30, 2011

Related U.S. Application Data
(60) Provisional application No. 61/428,975, filed on Dec. 31, 2010.

(51) Int. Cl. A61K 39/00; A61K 31/7105; A61K 9/00; A61K 39/35; A61K 39/12; A61K 39/02; A61K 39/385; A61P 35/00; A61P 31/00; A61P 3/00; A61P 25/28; A61P 37/00; A61P 29/00; C12N 5/02; C12Q 1/02; C07H 21/02; B82Y 5/00

(52) U.S. Cl. 424/400; 536/23.72; 536/24.5; 514/44 R; 424/184.1; 424/275.1; 424/204.1; 424/234.1; 424/193.1; 435/375; 435/29; 977/906; 977/795

(57) ABSTRACT

Immunostimulatory compositions include an isolated nucleic acid molecule that includes one or more nucleotide sequences from 5′- or 3′-terminal regions of positive-sense, single-stranded RNA virus genomes and/or or nucleotide sequences from a 5′-terminal regions of negative-sense, single-stranded RNA virus genomes.

### CROSS REFERENCE TO RELATED APPLICATIONS

Under 35 U.S.C. §119(e)(1), this application claims the benefit of prior U.S. provisional application 61/428,975, filed Dec. 31, 2010, the entire contents of which are hereby incorporated by reference.

### TECHNICAL FIELD

The invention relates to immunostimulatory nucleic acid compositions and methods of use therefor. More specifically, the invention relates to immunostimulatory viral RNA sequences, variants and conjugates thereof, and their use.

### BACKGROUND

Toll-like receptors (TLRs) are multi-domain proteins expressed by antigen-presenting cells (APCs) of the immune system. These proteins are capable of sensing so-called pathogen-associated molecular patterns (PAMPs), molecular features that signal an organism's invasion by bacteria, viruses, etc. PAMP binding by TLRs induces a number of cellular pathways, ultimately leading to the activation of an immune response. TLR-driven recognition of PAMPs is genetically-encoded and is not pathogen-specific, but is aimed at broad classes of pathogens, e.g., gram-negative bacteria, RNA viruses, etc. Thus, it constitutes an integral part of an innate immune response (compared to an adaptive immune response, which is directed against a specific pathogen and develops throughout an organism's life-span by means of natural or artificial immunization). Moreover, if a strong induction of an innate immune response is coupled with exposure to a specific antigen, this leads to a stronger adaptive response against said antigen.

Therefore, various TLR agonists are being developed as molecular adjuvants. Of these, single-stranded RNA (ssRNA) sequences are known to be capable of activating TLR7 and TLR8 with a variety of artificial and natural sequences of this sort described in the literature (Dieblod et al., 2004, Science, 303:1529-31; Heil et al., 2004, Science, 303:1526-29; and Forsbach et al., 2008, J. Immunol., 180:3729-38). However, there is a need for additional compositions and methods for activating TLR7 and TLR8.

### SUMMARY

We have discovered that nucleic acid molecules that include sequences from the 5′- or 3′-terminal region of positive-sense, single-stranded RNA virus genomes or sequences from the 5′-terminal region of negative-sense, single-stranded RNA virus genomes are capable of activating TLR7 and/or TLR8.

Accordingly, in one aspect this disclosure features immunostimulatory compositions that include an isolated nucleic acid molecule having a nucleotide sequence derived from a 5′- or 3′-terminal region (e.g., the first 10, 20, 30, 40, 50, 60, 70, 80, 90, or 100 bases from the 5′- or 3′-terminus) of a positive-sense, single-stranded RNA virus genome or from a 5′-terminal region (e.g., the first 10, 20, 30, 40, 50, 60, 70, 80, 90, or 100 bases from the 5′-terminus) of a negative-sense, single-stranded RNA virus genome. In some embodiments, the isolated nucleic acid molecule includes 10 to 80 (e.g., 10 to 70, 10 to 60, 10 to 50, 10 to 40, 10 to 30, 10 to 20, or 10) bases derived from the terminal region of the single-stranded RNA virus genome. In some embodiments, the isolated nucleic acid molecule is 10 to 200 nucleotides (e.g., 10 to 100, 10 to 80, 10 to 70, 10 to 60, 10 to 50, 10 to 40, 10 to 30, 10 to 20, or 10 nucleotides) in length.

Also provided are compositions (e.g., immunostimulatory compositions) containing an isolated nucleic acid molecule 10 to 200 nucleotides (e.g., 10 to 100, 10 to 80, 10 to 70, 10 to 60, 10 to 50, 10 to 40, 10 to 30, 10 to 20, or 10 nucleotides) in length containing the sequence of any one of SEQ ID NOs: 1-19, and compositions containing a nucleic acid of the sequence of any one of SEQ ID NOs: 1-19 (shown in Table 1 below).

TABLE 1
Exemplary nucleotide sequences derived from a 5′- or 3′-terminal region of a
positive-sense, single-stranded RNA virus genome or from a 5′-terminal region of a
negative-sense, single-stranded RNA virus genome

| SEQ ID NO: | Sequence | Nickname | Sequence derived from: |
|---|---|---|---|
| 1 | GAGAUGUUAUUUUGUUUU | SL-0001 | 3′-terminus of Chikungunya virus |
| | UAAUAUUUC | | strain TSI-GSD-218 (corresponding |
| | | | to genomic sequence bases 12010-12036) |
| 2 | AAGAAGAAAUAGAUUUAU | SL-0005 | 5′-terminus of Ebola virus Zaire |
| | UUUUAAAUUUUUGUGU | | strain (corresponding to genomic |
| | | | sequence bases 14-47) |
| 3 | GUUUAUCUGUGUGAACGA | SL-0004 | 5′-terminus of Japanese encephalitis |
| | UAGUGCAGUUUAAAC | | virus (corresponding to genomic |
| | | | sequence bases 5-20, 45-46, and 58-71) |
| 4 | GUUUAUCUGUGUGAACGA | SL-0017 | 5′-terminus of Japanese encephalitis |
| | UAGU | | virus (corresponding to a sequence |
| | | | present in SEQ ID NO: 3) |
| 5 | GUUUAUCUGUGUG | SL-0018 | 5′-terminus of Japanese encephalitis |
| | | | virus (corresponding to a sequence |
| | | | present in SEQ ID NO: 3) |
| 6 | CUGUGUGAACGAUAGUGC | SL-0019 | 5′-terminus of Japanese encephalitis |
| | AG | | virus (corresponding to a sequence |
| | | | present in SEQ ID NO: 3) |
| 7 | GUUUAUCUGUGUGCAGUU | SL-0020 | 5′-terminus of Japanese encephalitis |
| | UAAAC | | virus (corresponding to a sequence |
| | | | present in SEQ ID NO: 3) |
| 8 | UUUUUUGGAGCUUUUGAU | SL-0002 | 5′-terminus of Murray valley |
| | UUCAAAUG | | encephalitis virus (corresponding to |
| | | | genomic sequence bases 73-98) |
| 9 | UUUUUUGGAGCUUUUGAU | SL-0015 | 5′-terminus of Murray valley |
| | UU | | encephalitis virus (corresponding to |
| | | | genomic sequence bases 73-92) |
| 10 | UUUGGAGCUUUUGAUUUC | SL-0016 | 5′-terminus of Murray valley |
| | AA | | encephalitis virus (corresponding to |
| | | | genomic sequence bases 76-95) |
| 11 | UUAUUUUGUUUUUAAUAU | SL-0011 | 5′-terminus of Murray valley |
| | UUC | | encephalitis virus (corresponding to |
| | | | a sequence present in SEQ ID NO: 10) |
| 12 | UUAUUUUGUUUUUAAUAU | SL-0012 | 5′-terminus of Murray valley |
| | UU | | encephalitis virus (corresponding to |
| | | | a sequence present in SEQ ID NO: 10) |
| 13 | UGUUAUUUUGUUUUUAAU | SL-0013 | 5′-terminus of Murray valley |
| | AU | | encephalitis virus (corresponding to |
| | | | a sequence present in SEQ ID NO: 10) |
| 14 | AGAUGUUAUUGUUUAAUA | SL-0014 | 5′-terminus of Murray valley |
| | UUU | | encephalitis virus (corresponding to |
| | | | a sequence present in SEQ ID NO: 10) |
| 15 | AGAAAUAGAUUUAUUUUU | SL-0021 | 5′-terminus of Ebola virus Zaire |
| | | | strain (corresponding to a sequence |
| | | | present in SEQ ID NO: 2) |
| 16 | ACAAAAAAGAAUAAAUUU | SL-0022 | 5′-terminus of Ebola virus Zaire |
| | GUGU | | strain (corresponding to genomic |
| | | | sequence bases 8-18 and 35-45) |
| 17 | AUUUAUUUUUAAAUUUUU | SL-0023 | 5′-terminus of Ebola virus Zaire |
| | GUGU | | strain (corresponding to a sequence |
| | | | present in SEQ ID NO: 2) |
| 18 | ACACAAAAAAGAUUUUUG | SL-0024 | 5′-terminus of Ebola virus Zaire |
| | UGU | | strain (corresponding to genomic |
| | | | sequence bases 6-17 and 39-47) |
| 19 | UGGACACACAAAAAAGAA | SL-0025 | 5′-terminus of Ebola virus Zaire |
| | G | | strain (corresponding to genomic |
| | | | sequence bases 1-19) |

In some embodiments, the nucleic acid molecules are at least partially double-stranded. In some embodiments, the nucleic acid molecules are completely double-stranded.

In some embodiments, the nucleic acid molecules have a stabilized backbone. For example, the stabilized backbone can include at least one (e.g., at least two, three, four, or five) phosphorothioate internucleoside linkage, e.g., a complete phosphorothioate backbone, or at least one (e.g., at least two, three, four, or five) pyrophosphate internucleoside linkage, e.g., a complete pyrophosphate backbone.

In some embodiments, the nucleic acid molecules include at least one (e.g., at least two, three, four, or five) deoxyribonucleotide. In some embodiments, the nucleic acid molecules are ribonucleic acids (RNAs).

In some embodiments, the nucleic acid molecule is a Toll-like receptor (TLR) agonist, e.g., a TLR7 or TLR8 agonist.

In some embodiments, the positive-sense, single-stranded RNA virus is a member of the family Flaviviridae, e.g., a flavivirus (e.g., dengue, West Nile, yellow fever, tick-borne encephalitis, Japanese encephalitis, Murray Valley encephalitis, St. Louis encephalitis, Powassan, or Modoc virus), pestivirus, or hepatitis C virus.

In some embodiments, the positive-sense, single-stranded RNA virus is a member of the family Picornaviridae, e.g., an enterovirus (e.g., a poliovirus, Coxsackie virus, or echovirus), rhinovirus, cardiovirus, or hepatitis A virus.

In some embodiments, the positive-sense, single-stranded RNA virus is a member of the family Caliciviridae.

In some embodiments, the positive-sense, single-stranded RNA virus is a member of the family Togaviridae, e.g., an alphavirus (e.g., a Sindbis virus, Semliki Forest virus, Eastern equine encephalitis virus, Western equine encephalitis virus, Venezuelan equine encephalitis virus, Ross River virus, Chikungunya virus, Getah virus, Mayaro virus, or O′ nyong-nyong virus) or rubella virus.

In some embodiments, the positive-sense, single-stranded RNA virus is a member of the family Coronaviridae, e.g., a severe acute respiratory syndrome coronavirus (SARS-CoV).

In some embodiments, the positive-sense, single-stranded RNA virus is a member of the family Astroviridae, e.g., a human astrovirus.

In some embodiments, the negative-sense, single-stranded RNA virus is a member of the family Filoviridae, e.g., an Ebola virus or a Marburg virus.

In some embodiments, the negative-sense, single-stranded RNA virus is a member of the family Arenaviridae, e.g., a Lassa virus or lymphocytic choriomeningitis virus.

In some embodiments, the negative-sense, single-stranded RNA virus is a member of the family Bunyaviridae, e.g., a hantavirus, nairovirus (e.g., Crimean-Congo hemorrhagic fever virus or Dugbe virus), or orthobunyafirus (e.g., California encephalitis virus).

In some embodiments, the negative-sense, single-stranded RNA virus is a member of the family Bornaviridae.

In some embodiments, the negative-sense, single-stranded RNA virus is a member of the family Paramyxoviridae, e.g., a Nipah virus, canine distemper virus, measles virus, Rinderpest virus, Sendai virus, mumps virus, or respiratory syncytial virus.

In some embodiments, the negative-sense, single-stranded RNA virus is a member of the family Rhabdoviridae, e.g., a rabies virus.

In some embodiments, the negative-sense, single-stranded RNA virus is a member of the family Orthomyxoviridae, e.g., an influenza A virus, influenza B virus, influenza C virus, isavirus, or Thogoto virus.

In some embodiments, the composition comprises a condensing agent, e.g., a cationic lipid.

In some embodiments, the immunostimulatory composition can be an antigen or includes an antigen. For example, the nucleic acid can be admixed with an antigen.

In some embodiments, the immunostimulatory compositions can be a carrier or includes a carrier, e.g., a protein carrier, liposome, virosome, virus-like particle, or synthetic nanocarrier (e.g., a synthetic nanocarrier that includes a biodegradable polymer), and/or one or more (e.g., at least two, three, four, five, or six) antigens (e.g., a tumor-associated antigen). In some embodiments, the nucleic acid molecule is coupled, covalently or noncovalently, to the surface of the carrier (e.g., a synthetic nanocarrier). In some embodiments, the nucleic acid molecule is encapsulated within the carrier (e.g., a synthetic nanocarrier). In some embodiments, the carrier includes an antigen (e.g., a tumor-associated antigen) encapsulated within the carrier or coupled, covalently or noncovalently, to the surface of the carrier.

In another aspect, the disclosure features methods for stimulating an immune response, e.g., in a mammalian subject, such as a human subject. The methods include contacting a cell of the immune system with an amount of a composition described herein, e.g., in an amount effective to stimulate an immune response. In some embodiments, the methods are performed in vitro. In some embodiments, the methods are performed in vivo.

The disclosure also features immunostimulatory methods, that include obtaining one or more of any of the compositions described herein; and contacting an immune cell with the composition in an amount effective to immunostimulate the cell. For example, the immunostimulated immune cell can express a type-1 interferon, interferon-γ, tumor necrosis factor α, interleukin-6, interleukin-12, interleukin-10, or interleukin-23.

In another aspect, the disclosure features methods for stimulating a Th1-like immune response (e.g., expression of a type 1 interferon or interferon-γ) and/or expression of one or more of interleukin 12 (IL-12), IL-10, and IL-23) that include contacting a cell of the immune system with an amount of a composition described herein (e.g., any of the compositions described herein), e.g., in an amount effective to stimulate a Th1-like immune response. In some embodiments, the methods are performed in vitro. In some embodiments, the methods are performed in vivo.

In another aspect, the disclosure features methods for stimulating a proinflammatory immune response (e.g., inducing the expression of one or more proinflammatory cytokines, e.g., TNF-α and IL-6) that include contacting a cell of the immune system with an amount of a composition described herein (e.g., any of the compositions described herein), e.g., in an amount effective to stimulate a proinflammatory immune response. In some embodiments, the methods are performed in vitro. In some embodiments, the methods are performed in vivo.

In another aspect, the disclosure features methods of treating or reducing the risk of developing a cancer in a subject (e.g., a human). These methods include administering to a subject (e.g., a subject diagnosed with cancer or identified as having an increased risk of developing cancer) at least one dose (e.g., at least two, three, four, or five doses) of any of the compositions described herein including at least one tumor-associated antigen, in an amount effective to treat or reduce the risk of a cancer in the subject.

In another aspect, the disclosure features methods for stimulating TLR signaling that include contacting a cell expressing a TLR (e.g., a TLR7 or TLR8) with an amount of a composition described herein (e.g., any of the compositions described herein), e.g., in an amount effective to stimulate signaling by the TLR. In some embodiments, the methods are performed in vitro. In some embodiments, the methods are performed in vivo.

In another aspect, the disclosure features methods for stimulating an immune response in a subject (e.g., a human) that include administering to a subject an amount of a composition described herein (e.g., any of the compositions described herein), e.g., in an amount effective to stimulate an immune response in the subject.

In another aspect, the disclosure features methods for stimulating a Th1-like immune response in a subject (e.g., a human) that include administering to a subject an amount of a composition described herein (e.g., any of the compositions described herein), e.g., in an amount effective to stimulate a Th1-like immune response in the subject.

In another aspect, the disclosure features methods for stimulating a proinflammatory immune response in a subject (e.g., a human subject) that include administering to a subject an amount of a composition described herein (e.g., any of the compositions described herein), e.g., in an amount effective to stimulate a proinflammatory immune response in the subject.

In another aspect, the disclosure features methods for stimulating an antigen-specific immune response in a subject (e.g., a human), wherein the methods include administering to a subject a composition described herein (e.g., any of the compositions described herein) that includes an antigen (e.g., an allergen, viral antigen, bacterial antigen, hapten, or an antigen autologous or allogeneic to the subject) in an amount effective to stimulate an antigen-specific immune response in the subject.

In another aspect, the disclosure features methods for screening for an antagonist of a TLR (e.g., TLR7 or TLR8). These methods include: contacting a reference cell expressing a TLR with an amount of a composition described herein (e.g., any of the compositions described herein), in the absence of a candidate antagonist of the TLR, and measuring a reference amount of signaling by the TLR; contacting a test cell expressing the TLR with an amount of the composition, in the presence of the candidate antagonist of the TLR, and measuring a test amount of signaling by the TLR; and identifying the candidate antagonist of the TLR as an antagonist of the TLR when the reference amount of signaling exceeds the test amount of signaling.

In another aspect, the disclosure features compositions as described herein for stimulating an immune response (e.g., a Th1-like immune response or a proinflammatory immune response) in a subject.

In another aspect, the disclosure features a combination of a composition as described herein and an antigen for stimulating an antigen-specific immune response in a subject.

In another aspect, the disclosure features combinations of the compositions described herein and one or more tumor-associated antigens for use in methods of treating or of reducing the risk of developing cancer in a subject (e.g., a human).

Also provided are methods of making immunostimulatory compositions. These methods include: (a) isolating at least one 10 to 40 nucleotide sequence from the first 80 bases from a 5′- or 3′-terminus of a positive-sense single-stranded RNA virus genome, or at least one 10 to 40 nucleotide sequence from the first 80 bases from a 5′-terminus of a negative-sense single-stranded RNA virus genome that has immunostimulatory activity; and (b) mixing the at least one isolated 10 to 40 nucleotide sequence from the first 80 bases from a 5′- or 3′-terminus of a positive-sense single-stranded RNA virus genome, or the at least one isolated 10 to 40 nucleotide sequence from the first 80 bases from a 5′-terminus of a negative-sense single-stranded RNA virus genome with a carrier or a pharmaceutically acceptable excipient (e.g., phosphate buffered saline).

Also provided are methods of administering a composition of one or more of any of the immunostimulatory compositions described herein to a subject. In some embodiments, the administering can occur in two or more doses. In some embodiments, the subject has a disease or disorder. In some embodiments, the disease or disorder is selected from the group of: a cancer, an infectious disease, a metabolic disease, a degenerative disease, an autoimmune disease, an inflammatory disease, or an immunological disease. In some embodiments, the infectious disease can be a viral, bacterial, parasitic, or fungal infection. In some embodiments, where the subject has a disease or disorder, the amount of the composition administered is effective to reduce the number of symptoms of the disease or disorder experienced by the subject; reduce the severity, frequency, or duration of one or more symptoms of the disease or disorder in the subject; and/or improve the therapeutic outcome in the subject (e.g., reduce a tumor size, reduce a number of tumor, bacterial, or other pathogenic cells circulating in the blood (e.g., reduce circulating tumor cells (CTCs)), or reduce a viral or bacterial load).

The immunostimulatory compositions provided herein are capable of significantly inducing an immune response (e.g., a Th1-like immune response, a proinflammatory immune response, or an antigen-specific immune response as described herein) in a subject that has been administered at least one dose of at least one of the immunostimulatory compositions described herein (e.g., at least a 10% increase (e.g., at least a 20%, 30%, 40%, 50%, 60%, 70%, 80%, 90%, 100%, 110%, 120%, 130%, 150%, or 200% increase) in the level of one or more measurable physical parameters of an immune response in a subject (e.g., an increase in the level of one or more cytokines selected from a type 1 interferon, interferon-γ, tumor necrosis factor α, interleukin-6, interleukin-12, interleukin-10, and interleukin-23) as compared to the level of the immune response (e.g., the level of one or more measurable physical parameters of an immune response) in the subject prior to administration of the at least one dose of at least one immunostimulatory composition described herein or the level of the immune response in a control subject not receiving a treatment or receiving a different treatment (e.g., a placebo or control scrambled nucleic acid).

The immunostimulatory compositions provided herein can also result in a significant decrease (e.g., at least a 10%, 20%, 30%, 40%, 50%, 60%, 70%, 80%, or 90% decrease) in a tumor mass present in a subject having cancer, a decrease (e.g., a significant or observable decrease) in the severity, frequency, or duration of one or more symptoms of cancer, a decrease in the number of symptoms of cancer, and/or a decrease (e.g., a significant, observable, or detectable decrease) in the rate of tumor growth in a subject that is administered at least one dose of at least one of the immunostimulatory compositions described herein (e.g., as compared to the same physical parameter(s) observed in the same subject prior to administration of the at least one dose of at least one immunostimulatory composition or in a control subject having the same cancer, but not receiving a treatment or receiving a different treatment (e.g., a placebo or control scrambled nucleic acid). In some embodiments, the administration of at least one dose of at least one immunostimulatory composition provided herein can significantly improve the prognosis of a subject having cancer (e.g., an increased longevity of life after administration of the at least one dose of at least one immunostimulatory composition provided herein) (e.g., as compared to the prognosis of a subject having cancer, but not receiving a treatment or receiving a different treatment (e.g., a placebo or control scrambled nucleic acid).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patents and patent applications cited herein, whether supra or infra, are hereby incorporated by reference in their entirety for all purposes. In case of conflict, the present specification, including definitions, will control. In addition, the materials, methods, and examples are illustrative only and not intended to be limiting.

It is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

Other features and advantages of the invention will be apparent from the following detailed description, and from the claims.

### DESCRIPTION OF DRAWINGS

FIG. 1 is a line graph depicting activation of TLR8-driven transcription by ribonucleotides SL-0001 and SL-0005 (shown in Table 1). Serial dilutions of test and control oligonucleotides were complexed with DOTAP (N-[1-(2,3-dioleoyloxy)propyl]-N,N,N-trimethyl-ammonium methylsulfate) and used for transfection of reporter HEK-Blue™ hTLR8 cells. Secreted embryonic alkaline phosphatase (SEAP) activity was measured in culture media at 40 hours after transfection.

FIG. 2 is a line graph depicting TNF-α induction by ribonucleotides SL-0001 and SL-0005. J774 murine macrophages (50,000 cells/well) were treated with serial dilutions of DOTAP-complexed oligonucleotides and the levels of TNF-α in culture medium measured at 18 hours after transfection.

FIG. 3A is a line graph depicting activation of TLR8-driven transcription by ribonucleotides SL-0004, SL-0017, SL-0018, SL-0019, SL-0020, and R-0006. Serial dilutions of test and control oligonucleotides were complexed with DOTAP and used for transfection of reporter HEK-Blue™ hTLR8 cells. Secreted embryonic alkaline phosphatase (SEAP) (reporter gene) activity was measured in culture media at 40 hours after transfection.

FIG. 3B is a line graph depicting activation of TLR8-driven transcription by ribonucleotides SL-0002, SL-0015, and SL-0016. Serial dilutions of test oligonucleotides were complexed with DOTAP and used for transfection of reporter HEK-Blue™ hTLR8 cells. SEAP activity was measured in culture media at 40 hours after transfection.

FIG. 4 is a line graph depicting TNF-α induction by ribonucleotides SL-0004, SL-0017, SL-0018, SL-0019, and SL-0020. J774 murine macrophages (50,000 cells/well) were treated with serial dilutions of DOTAP-complexed oligonucleotides and the levels of TNF-α in culture medium were measured at 24 hours after transfection.

FIG. 5A is a line graph depicting activation of TLR8-driven transcription by ribonucleotides SL-0001, SL-0011, SL-0012, SL-0013, SL-0014, and R-0006. Serial dilutions of test and control oligonucleotides were complexed with DOTAP and used for transfection of reporter HEK-Blue™ hTLR8 cells. SEAP activity was measured in culture media at 40 hours after transfection.

FIG. 5B is a line graph depicting activation of TLR8-driven transcription by ribonucleotides SL-0005, SL-0021, SL-0022, SL-0023, SL-0024, SL-0025, and R-0006. Serial dilutions of test and control oligonucleotides were complexed with DOTAP and used for transfection of reporter HEK-Blue™ hTLR8 cells. SEAP activity was measured in culture media at 40 hours after transfection.

FIG. 6A is a graph showing the levels of TNF-α and IL-6 secreted by murine lymphocytes (10⁶ cells/well) following no treatment (intact), treatment with DOTAP alone (DOTAP), TLR7/8 agonist (R848; 1 μM), or DOTAP-complexed ribonucleotide SL-0001, SL-0011, SL-0012, SL-0014, SL-0005, SL-0021, or SL-0023 (200 nm). The levels of TNF-α and IL-6 were measured in the culture media using ELISA specific for TNF-α and IL-6 following overnight incubation.

FIG. 6B is a graph showing the levels of IL-12 and IFN-γ secreted by murine lymphocytes (10⁶ cells/well) following no treatment (intact), treatment with DOTAP alone (DOTAP), TLR7/8 agonist (R848; 1 μM), or with DOTAP-complexed ribonucleotide SL-0001, SL-0011, SL-0012, SL-0014, SL-0005, SL-0021, or SL-0023 (200 nm). The levels of IL-12 and IFN-γ were measured in the culture media using ELISA specific for IL-12 and IFN-γ following overnight incubation.

FIG. 7A is a graph showing the percentage of CD69⁺ macrophages, plasmacytoid dendritic cells (pDC), and B-cells present in a population of murine splenocytes following treatment with R848 (1 μM), DOTAP alone, or DOTAP-complexed ribonucleotide SL-0001, SL-0011, SL-0012, SL-0014, or R-0008 (200 nm) for 20 hours. The percentage of CD69⁺ cells present in the population was determined using fluorescence-assisted cell sorting (FACS).

FIG. 7B is a graph showing the percentage of CD69⁺ natural killer (NK) cells and myeloid dendritic cells (mDC) present in a population of murine splenocytes following treatment with R848 alone (1 μM), DOTAP alone, or DOTAP-complexed ribonucleotide SL-0001, SL-0011, SL-0012, SL-0014, or R-0008 (200 nm) for 20 hours. The percentage of CD69⁺ cells present in the population was determined using fluorescence-assisted cell sorting (FACS).

FIG. 7C is a graph showing the percentage of CD69⁺ granulocytes, T cells (CD3⁺ CD69⁺ high cells), and natural killer T (NKT) cells present in a population of murine splenocytes following treatment with R848 alone (1 μM), DOTAP alone, or DOTAP-complexed ribonucleotide SL-0001, SL-0011, SL-0012, SL-0014, or R-0008 (200 nm) for 20 hours. The percentage of CD69⁺ cells present in the population was determined using FACS.

FIG. 8A is a graph showing the levels of TNF-α and IL-6 secreted by primary human lymphocytes (10⁶ cells/well) following treatment with DOTAP alone, R848 (1 μM), or DOTAP-complexed ribonucleotide SL-0001, ST-0005, or R-0006 (200 nm). The levels of TNF-α and IL-6 were measured in the culture media using Luminex assays (Aushon BioSystems, Billerica, Mass.) following 20-hour incubation.

FIG. 8B is a graph showing the levels of interferon-γ, IL-10, IL-12 (p40), and IL-23 secreted by primary human lymphocytes (10⁶ cells/well) following treatment with DOTAP alone, R848 (1 μM), or DOTAP-complexed ribonucleotide SL-0001, ST-0005, or R-0006 (200 nm). The levels of interferon-γ, IL-10, IL-12, and IL-23 were measured in the culture media using Luminex assays (Aushon BioSystems, Billerica, Mass.) following 20-hour incubation.

### DETAILED DESCRIPTION

### Introduction

This disclosure describes the discovery that certain nucleic acid sequences present in generally highly conserved regions of genomic RNA of certain RNA viruses are highly immunostimulatory. More specifically, the discovery is that sequences found near the 3′ and 5′ termini of single-stranded positive-sense RNA virus genomic RNA molecules and sequences near the 5′ terminus of negative-sense RNA virus genomic RNA molecules are immunostimulatory. Furthermore, the nucleic acid molecules described herein act as agonists for signaling by certain TLRs. The nucleic acid molecules described herein are potent inducers of Th1-like and proinflammatory immune responses, and thus are useful for directing an immune response toward a Th1-like or proinflammatory immune response and for methods of therapy that harness these effects.

### DEFINITIONS

“Adjuvant” means an agent that does not constitute a specific antigen, but boosts the strength and/or longevity of immune response to a concomitantly administered antigen. Such adjuvants may include, but are not limited to, stimulators of pattern recognition receptors, such as Toll-like receptors, retinoic acid-inducible gene-1 (RIG-1) and nucleotide-binding oligomerization domain (NOD)-like receptors (NLR), mineral salts, such as alum, alum combined with monphosphoryl lipid (MPL) A of Enterobacteriaceae, such as Escherichia coli, Salmonella minnesota, Salmonella typhimurium, or Shigella flexneri or specifically with MPL® (AS04), MPL A of above-mentioned bacteria separately, saponins, such as QS-21, Quil-A, ISCOMs, ISCOMATRIX™, emulsions such as MF59™, Montanide® ISA 51 and ISA 720, AS02 (QS21+squalene+MPL®), liposomes and liposomal formulations, such as AS01, synthesized or specifically prepared microparticles and microcarriers, such as bacteria-derived outer membrane vesicles (OMV) of Neisseria meningitidis, N. gonorrheae, Francisella novicida and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments.

In some embodiments, adjuvants include agonists for pattern recognition receptors (PRR), including, but not limited to Toll-Like Receptors (TLRs), specifically TLRs 2, 3, 4, 5, 7, 8, or 9 and/or combinations thereof. In other embodiments, adjuvants include agonists for Toll-Like Receptors 3, agonists for Toll-Like Receptors 7 and 8, or agonists for Toll-Like Receptor 9; e.g., the recited adjuvants include imidazoquinolines; such as R848; adenine derivatives, such as those disclosed in U.S. Pat. No. 6,329,381 (Sumitomo Pharmaceutical Company) (herein incorporated by reference); immunostimulatory DNA; or immunostimulatory RNA. In some embodiments, synthetic nanocarriers incorporate as adjuvants compounds that are agonists for toll-like receptors (TLRs) 7 and 8 (“TLR 7/8 agonists”). In some embodiments, the TLR 7/8 agonist compounds are those disclosed in U.S. Pat. No. 6,696,076 (herein incorporated by reference), including but not limited to, imidazoquinoline amines, imidazopyridine amines, 6,7-fused cycloalkylimidazopyridine amines, and 1,2-bridged imidazoquinoline amines. In some embodiments, the adjuvants include imiquimod and resiquimod (also known as R848). In some embodiments, an adjuvant may be an agonist for the DC surface molecule CD40. In some embodiments, to stimulate immunity rather than tolerance, a synthetic nanocarrier incorporates an adjuvant that promotes DC maturation (needed for priming of naive T cells) and the production of cytokines, such as type I interferons, which promote antibody immune responses.

In some embodiments, adjuvants also can include immunostimulatory RNA molecules, such as, but not limited to, dsRNA or poly I:C (a TLR3 stimulant), and/or those disclosed in Heil et al., 2004, Science, 303:1526-29; Vollmer et al., WO 2008/033432; Forsbach et al., WO 2007/062107; Uhlmann et al., US 2006/0241076; Lipford et al., WO 2005/097993 A2; Lipford et al., WO 2003/086280 (each of which is herein incorporated by reference). In some embodiments, an adjuvant can be a TLR-4 agonist, such as bacterial lipopolysacccharide (LPS), VSV-G, and/or HMGB-1. In some embodiments, adjuvants can include TLR-5 agonists, such as flagellin, or portions or derivatives thereof, including, but not limited to, those disclosed in U.S. Pat. Nos. 6,130,082, 6,585,980, and 7,192,725 (each of which is herein incorporated by reference). In some embodiments, synthetic nanocarriers incorporate a ligand for Toll-like receptor (TLR)-9, such as immunostimulatory DNA molecules comprising CpGs, which induce type I interferon or interferon-γ secretion, and stimulate T and B cell activation leading to increased antibody production and cytotoxic T cell responses (Krieg et al., 1995, Nature, 374:546-549; Chu et al., 1997, J. Exp. Med., 186:1623-31; Lipford et al., 1997, Eur. J. Immunol., 27:2340-44; Roman et al., 1997, Nat. Med., 3:849-854; Davis et al., 1998, J. Immunol., 160:870-876; Lipford et al., 1998, Trends Microbiol., 6:496-500; and U.S. Pat. Nos. 6,207,646; 7,223,398; 7,250,403; or 7,566,703 (each of which is incorporated by reference)).

In some embodiments, adjuvants can be proinflammatory stimuli released from necrotic cells (e.g., urate crystals). In some embodiments, adjuvants may be activated components of the complement cascade (e.g., CD21, CD35, etc.). In some embodiments, adjuvants can be activated components of immune complexes. The adjuvants also include complement receptor agonists, such as a molecule that binds to CD21 or CD35. In some embodiments, the complement receptor agonist induces endogenous complement opsonization of the synthetic nanocarrier. In some embodiments, adjuvants are cytokines, which are small proteins or biological factors (in the range of 5 kD-20 kD) that are released by cells and have specific effects on cell-cell interaction, communication, and/or behavior of other cells. In some embodiments, the cytokine receptor agonist is a small molecule, antibody, fusion protein, or aptamer.

In some embodiments, at least a portion of the dose of adjuvant can be coupled to synthetic nanocarriers, e.g., all of the dose of adjuvant is coupled to a synthetic nanocarrier. In other embodiments, at least a portion of the dose of the adjuvant is not coupled to a synthetic nanocarrier. In some embodiments, the dose of adjuvant includes two or more types of adjuvants. For instance, and without limitation, adjuvants that act on different TLR receptors can be combined. As an example, in some embodiments, a TLR 7/8 agonist can be combined with a TLR 9 agonist. In some embodiments, a TLR 7/8 agonist can be combined with a TLR 4 agonist. In some embodiments, a TLR 9 agonist can be combined with a TLR 3 agonist.

“Administering a drug” or “administration of a drug” means providing a drug to a subject in a manner that is pharmacologically useful.

“Antigen” means a B cell antigen or T cell antigen. In some embodiments, antigens are coupled to synthetic nanocarriers. In some embodiments, antigens are not coupled to the synthetic nanocarriers. In some embodiments, antigens are co-administered with the synthetic nanocarriers. In some embodiments, antigens are not co-administered with the synthetic nanocarriers. “Type(s) of antigens” means molecules that share the same, or substantially the same, antigenic characteristics.

“B cell antigen” means any antigen that is, or is recognized by, and triggers an immune response in a B cell (e.g., an antigen that is specifically recognized by a B cell receptor on a B cell). In some embodiments, an antigen that is a T cell antigen is also a B cell antigen. In some embodiments, the T cell antigen is not also a B cell antigen. B cell antigens include, but are not limited to proteins, peptides, small molecules, and carbohydrates. In some embodiments, the B cell antigen includes a non-protein antigen (i.e., not a protein or peptide antigen). In some embodiments, the B cell antigen includes a carbohydrate associated with an infectious agent. In some embodiments, the B cell antigen includes a glycoprotein or glycopeptide associated with an infectious agent. The infectious agent can be a bacterium, virus, fungus, protozoan, or parasite. In some embodiments, the B cell antigen includes a poorly immunogenic antigen. In some embodiments, the B cell antigen includes an abused substance or a portion thereof. In some embodiments, the B cell antigen includes an addictive substance or a portion thereof. Addictive substances include, but are not limited to, nicotine, a narcotic, a cough suppressant, a tranquilizer, and a sedative. In some embodiments, the B cell antigen includes a toxin, such as a toxin from a chemical weapon or natural sources. The B cell antigen can also include a hazardous environmental agent. In some embodiments, the B cell antigen includes a self antigen. In other embodiments, the B cell antigen includes an alloantigen, an allergen, a contact sensitizer, a degenerative disease antigen, a hapten, an infectious disease antigen, a cancer antigen, an atopic disease antigen, an autoimmune disease antigen, an addictive substance, a xenoantigen, or a metabolic disease enzyme or enzymatic product thereof “Carrier” means a substance that can be co-administered with one or more immunostimulatory isolated nucleic acids (e.g., any of the immunostimulatory nucleic acids described herein), and that may alter in vivo and/or in vitro characteristics of the nucleic acids, such as pharmacokinetics, stability, and/or trafficking Carriers differ from pharmaceutically acceptable excipients in that pharmaceutically acceptable excipients include pharmacologically inactive materials, while carriers include materials that may alter the in vivo and/or in vivo characteristics of the immunostimulatory isolated nucleic acids. In some embodiments, carriers can include nanocarriers comprising poly(lactic-co-glycolic acid) (PLGA), poly(lactide-co-glycolide) PLG, poly(lactic acid) (PLA), poly(D,L-lactide), poly(D,L-glycolide) (PG), poly(lactide-co-glycolide) (PLG), poly(cyanoacrylate) (PCA), and/or silica; perfluorocarbon(s); lipids; gelatin; chitosan; and/or cyclodextrin. In some embodiments, carriers can also include proteins, such as albumin, collagen, or CRM197.

By the term “CRM197” or “cross-reacting material 197” is meant a nontoxic version of a diphtheria toxin protein that shares the immunological properties of the native molecule and contains a sequence that is at least 80% (e.g., at least 85%, 90%, 91%, 92%, 93%, 94%, 95%, 96%, 97%, 98%, or 99%) identical to the sequence present in a wild type diphtheria toxin protein.

“Coadministered” means administering two or more substances to a subject in a manner that is correlated in time. In some embodiments, coadministration can occur through administration of two or more (e.g., at least two, three, four, five, or six) substances in the same dosage form. In other embodiments, coadministration can encompass administration of two or more substances in different dosage forms, but within a specified period of time, e.g., within 1 month, e.g., within 1 week, e.g., within 1 day, or within 1 hour, 45 minutes, 30 minutes, 15 minutes, 10 minutes, 5 minutes, or 1 minute, or at the same, or about the same, time.

“Couple” or “Coupled” or “Couples” (and the like) means to chemically associate one entity (for example a moiety) with another. In some embodiments, the coupling is covalent, meaning that the coupling occurs in the context of the presence of a covalent bond between the two entities. In non-covalent embodiments, the non-covalent coupling is mediated by non-covalent interactions including, but not limited to, charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In some embodiments, encapsulation is a form of coupling.

“Derived” means taken from a source, e.g., a biological source, and subjected to modification. For example, a “derived” peptide or nucleic acid has a sequence with at least 50% identity (e.g., at least 55%, 60%, 65%, 70%, 75%, 80%, 85%, 90%, or 95% identity) to a natural peptide or nucleic acid, and has one or more altered chemical or immunological properties as compared to the natural peptide or nucleic acidThese chemical or immunological properties include, for example, hydrophilicity, stability, affinity, and ability to couple with a carrier such as a synthetic nanocarrier. In some embodiments, a derived peptide or nucleic acid is produced synthetically. In some embodiments, the modifications can be the insertion of one or more (e.g., at least one, two, three, four, five, six, seven, eight, nine, or ten) nucleotides or amino acids at one or both terminal ends or within the nucleic acid or polypeptide from a biological source, respectively; the deletion of one or more (e.g., at least one, two, three, four, five, six, seven, eight, nine, or ten) nucleotides or amino acids at one or both terminal ends or within the nucleic acid or polypeptide from a biological source; the addition of one or more (e.g., at least one, two, three, four, five, six, seven, eight, nine, or ten) nucleotides or amino acids at one or both terminal ends or within the nucleic acid or polypeptide from a biological source; and/or the substitution of one or more (e.g., at least one, two, three, four, five, six, seven, eight, nine, or ten) nucleotides (e.g., substitution with a modified nucleotide or base) anywhere within the nucleic acid or polypeptide from a biological source.

“Dosage form” means a pharmacologically and/or immunologically active material in a medium, carrier, vehicle, or device suitable for administration to a subject.

“Encapsulate” means to enclose, e.g., within a synthetic nanocarrier, e.g., enclose completely, e.g., within a synthetic nanocarrier. Most or all of a substance that is encapsulated is not exposed to the local environment external to the synthetic nanocarrier. Encapsulation is distinct from absorption, which places most or all of a substance on a surface, e.g., of a synthetic nanocarrier, and leaves the substance exposed to the local environment external to the surface, e.g., of the synthetic nanocarrier.

The term “fragment,” when referring to a polypeptide, means less than 75% of the sequence or mass of the wild type or purified polypeptide. In some embodiments, less than 70%, less than 65%, less than 60%, less than 55%, or less than 50%, of the mass or sequence of the wild type or purified polypeptide is present. In general the mass or sequence of a wild type or purified protein that is present in a fragment can be determined using conventional methods. In one embodiment, GPC-HPLC (gel permeation chromatography-high pressure liquid chromatography) can be used for determining the molecular weight of a glycosylated polypeptide, and the Lowry assay and a phenol-sulfuric acid assay can be used to determine the amount of the amino acid and saccharide material present in a polypeptide, respectively.

“Immunostimulatory” means that a substance has a stimulatory effect on a mammalian immune system. Such substances can be readily identified using standard assays that indicate various aspects of the immune response, such as cytokine secretion, antibody production, NK cell activation, and T cell proliferation. See, e.g., WO 97/28259; WO 98/16247; WO 99/11275; Krieg et al., 1995, Nature, 374:546-549; Yamamoto et al., 1992, J. Immunol., 148:4072-76; Ballas et al., 1996, J. Immunol., 157:1840-45; Klinman et al., 1997, J. Immunol., 158:3635-39; Sato et al., 1996, Science, 273:352-354; Pisetsky, 1996, J. Immunol., 156:421-423; Shimada et al., 1986, Jpn. J. Cancer Res., 77:808-816; Cowdery et al., 1996, J. Immunol., 156:4570-75; Roman et al., 1997, Nat. Med., 3:849-854; Lipford et al., 1997, Eur. J. Immunol., 27:2340-44; WO 98/55495; and WO 00/61151. For example, an immunostimulatory composition can induce a Th1-like immune response (e.g., the production or secretion of one or more IFN-γ, IL-10, IL-12, and IL-23) or a proinflammatory immune response (e.g., the production or secretion of one or more proinflammatory cytokines, e.g., TNF-α and/or IL-6). Accordingly, these and other methods can be used to identify, test, and/or confirm immunostimulatory substances, such as immunostimulatory nucleotides, e.g., any of the immunostimulatory isolated nucleic acids described herein.

“Immunostimulate” means to activate an immune cell, e.g., a mammalian immune cell, and/or induce or increase (e.g., a detectable or measurable increase) one or more (e.g., two, three, four, or five) biological activities in an immune cell, e.g., such as a mammalian immune cell, e.g., a human immune cell, which are associated with an immune response, e.g., in a mammal. Non-limiting examples of biological activities associated with an immune response include the production of antibodies (e.g., antibodies specific towards any of the antigens described herein, e.g., IgG, IgA, IgE, and/or IgM) and/or the secretion of one or more cytokines (e.g., one or more cytokines selected from the group of type 1 interferon, interferon-γ, tumor necrosis factor α, interleukin-6, interleukin-12, interleukin-10, and interleukin-23). Non-limiting examples of mammalian immune cells include macrophages (e.g., F4/80⁺/GR1⁻ cells), plasmacytoid dendritic cells (e.g., CD11c⁺/CD220⁺ cells), B cells (e.g., CD220⁺/CD11c⁻ cells), NK cells (e.g., CD3⁻/Ly49b+ cells), myeloid dendritic cells (e.g., CD11c⁺/CD220⁺ cells), granulocytes (eosinophils) (GR1⁺high/F4/80⁻), T cells (CD3⁺), and natural killer T cells (CD3⁺/Ly49b⁺). Additional examples of biological activities associated with an immune response and mammalian immune cells are known in the art. In addition, immunostimulation of a cell can occur in vitro or in vivo.

“Isolated nucleic acid” means a nucleic acid that is separated from its native environment and present in sufficient quantity to permit its identification or use. An isolated nucleic acid can be one that is (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one that is readily manipulable by recombinant DNA techniques known in the art. Thus, a nucleotide sequence contained in a vector in which 5′ and 3′ restriction sites are known or for which polymerase chain reaction (PCR) primer sequences have been disclosed is considered isolated, but a nucleic acid sequence existing in its native state in its natural host is not. An isolated nucleic acid can be purified (e.g., at least 60%, 70%, 80%, 90%, 92%, 93%, 94%, 95%, 96%, 97%, 98%, or 99% by weight), but need not be. For example, a nucleic acid that is isolated within a cloning or expression vector is not pure in that it can comprise only a tiny percentage of the material in the cell in which it resides. Such a nucleic acid is isolated, however, as the term is used herein, because it is readily manipulable by standard techniques known to those of ordinary skill in the art. Any of the nucleic acids described herein can be isolated.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheriodal synthetic nanocarrier, the maximum and minimum dimension of a synthetic nanocarrier would be substantially identical, and would be the size of its diameter. Similarly, for a cubiodal synthetic nanocarrier, the minimum dimension of a synthetic nanocarrier would be the smallest of its height, width, or length, while the maximum dimension of a synthetic nanocarrier would be the largest of its height, width, or length.

“Nucleic acid” in the context of the immunostimulatory nucleic acids described herein means a string of linked nucleotides or modified nucleotides. The sugar moieties can be ribose, deoxyribose, or any of the various modified sugars as described herein (including combinations thereof) or known in the art. The base moieties can be any purine or pyrimidine bases, including C, A, T, G, and U, and any of the modified bases as described herein (including combinations thereof). The nucleic acids can be linked by natural phosphodiester bonds, or by any of the other linkages described herein (including for example phosphorothioate links, so exhibiting a modified backbone), including combinations thereof. The nucleic acid can be single or double stranded, and can be of any topology/conformation (including branched, circular, and hairpin). In some embodiments, modification of the nucleic acids described herein with such modified sugars, bases, and/or backbones are stabilizing modifications, as described herein.

“Obtained” means taken from a source, e.g., a biological source, without modification. For example, as a non-limiting example, an obtained nucleic acid can taken from a biological source (e.g., a sequence taken from a 5′- or 3′-terminal region of positive-sense, single-stranded RNA virus genome or a sequence taken from a 5′-terminal region of negative-sense, single-stranded RNA virus genome) and have chemical and/or immunological properties that are not significantly different from the natural nucleic acid. These chemical or immunological properties include hydrophilicity, stability, affinity, and ability to couple with a carrier such as a synthetic nanocarrier.

“Pharmaceutically acceptable excipient” means a pharmacologically inactive material used together with the recited nucleic acids to formulate compositions that can be administered to a subject. Pharmaceutically acceptable excipients include a variety of materials known in the art, including, but not limited to, saccharides (such as glucose, lactose, and the like), preservatives such as antimicrobial agents, reconstitution aids, colorants, saline (such as phosphate buffered saline), and buffers.

“Subject” means humans and animals, including waiin blooded mammals, such as primates; avians; domestic household or farm animals, such as cats, dogs, sheep, goats, cattle, horses, and pigs; laboratory animals, such as mice, rats, and guinea pigs; fish; reptiles; zoo and wild animals; and the like.

“Synthetic nanocarrier(s)” means a discrete object that is not found in nature, and that possesses at least one dimension that is less than or equal to 5 microns in size.

A synthetic nanocarrier can be, but is not limited to, one or a plurality of lipid-based nanoparticles, polymeric nanoparticles, metallic nanoparticles, surfactant-based emulsions, dendrimers, buckyballs, nanowires, virus-like particles, peptide or protein-based particles (such as albumin nanoparticles), and/or nanoparticles that are developed using a combination of nanomaterials such as lipid-polymer nanoparticles. Synthetic nanocarriers can be a variety of different shapes, including but not limited to spheroidal, cuboidal, pyramidal, oblong, cylindrical, toroidal, and the like. Synthetic nanocarriers as described herein can include one or more surfaces. Exemplary synthetic nanocarriers that can be adapted for use in disclosed compositions and methods can include: (1) the biodegradable nanoparticles disclosed in U.S. Pat. No. 5,543,158, (2) the polymeric nanoparticles of U.S. Patent Application Publication No. 2006/0002852, (3) the lithographically constructed nanoparticles of U.S. Patent Application Publication No. 2009/0028910, (4) the disclosure of WO 2009/051837, and (5) the nanoparticles disclosed in U.S. Patent Application Publication No. 2008/0145441. In some embodiments, synthetic nanocarriers can possess an aspect ratio greater than 1:1, 1:1.2, 1:1.5, 1:2, 1:3, 1:5, or 1:7, or greater than 1:10. Albumin nanoparticles are generally included as synthetic nanocarriers, however in some embodiments, the synthetic nanocarriers do not include albumin nanoparticles. In some embodiments, the synthetic nanocarriers do not include chitosan.

Synthetic nanocarriers can have a minimum dimension of equal to or less than 100 nm, e.g., equal to or less than about 100 nm, can optionally not include a surface with hydroxyl groups that activate complement, or alternatively can include a surface that consists essentially of moieties that are not hydroxyl groups that activate complement. In some embodiments, synthetic nanocarriers have a minimum dimension of equal to or less than about 100 nm, e.g., equal to or less than about 100 nm, and do not include a surface that substantially activates complement or alternatively include a surface that consists essentially of moieties that do not substantially activate complement. In another embodiment, synthetic nanocarriers have a minimum dimension of equal to or less than about 100 nm, e.g., equal to or less than about 100 nm, and do not include a surface that activates complement or alternatively include a surface that consists essentially of moieties that do not activate complement. In some embodiments, synthetic nanocarriers exclude virus-like particles (VLPs). In some embodiments, when synthetic nanocarriers include VLPs, the VLPs include non-natural adjuvant (meaning that the VLPs include an adjuvant other than naturally occurring RNA generated during the production of the VLPs). In some embodiments, synthetic nanocarriers can possess an aspect ratio greater than 1:1, 1:1.2, 1:1.5, 1:2, 1:3, 1:5, or 1:7, or greater than 1:10.

“T cell antigen” means any antigen that is recognized by and triggers an immune response in a T cell (e.g., an antigen that is specifically recognized by a T cell receptor on a T cell or a natural killer T (NKT) cell via presentation of the antigen or portion thereof bound to a Class I or Class II major histocompatability complex (MHC) molecule, or bound to a cluster of differentiation 1 (CD1) complex. In some embodiments, an antigen that is a T cell antigen is also a B cell antigen. In other embodiments, the T cell antigen is not also a B cell antigen. T cell antigens generally are proteins or peptides. T cell antigens can be an antigen that stimulates a CD8⁺ T cell response, a CD4⁺ T cell response, or both. The nanocarriers, therefore, in some embodiments can effectively stimulate both types of responses.

In some embodiments the T cell antigen is a T helper cell antigen (i.e., one that can generate an enhanced response to a B cell antigen, e.g., an unrelated B cell antigen, through stimulation of T cell help). In some embodiments, a T helper cell antigen can include one or more peptides obtained or derived from tetanus toxoid, Epstein-Barr virus, influenza virus, respiratory syncytial virus, measles virus, mumps virus, rubella virus, cytomegalovirus, adenovirus, diphtheria toxoid, or a PADRE peptide (described in U.S. Pat. No. 7,202,351; herein incorporated by reference). In other embodiments, a T helper cell antigen can include one or more lipids, or glycolipids, including but not limited to: α-galactosylceramide (α-GalCer), α-linked glycosphingolipids (from Sphingomonas spp.), galactosyl diacylglycerols (from Borrelia burgdorferi), lypophosphoglycan (from Leishmania donovani), and phosphatidylinositol tetramannoside (PIM4) (from Mycobacterium leprae). For additional lipids and/or glycolipids useful as a T helper cell antigen, see V. Cerundolo et al., 2009, Nature Rev. Immun., 9:28-38. In some embodiments, CD4⁺ T-cell antigens can be derivatives of a CD4⁺ T-cell antigen that is obtained from a source, such as a natural source. In such embodiments, CD4⁺ T-cell antigen sequences, such as those peptides that bind to MHC II, can have at least 70%, 80%, 90%, or 95% identity to the antigen obtained from the source. In some embodiments, the T cell antigen, e.g., a T helper cell antigen, can be coupled to, or uncoupled from, a synthetic nanocarrier.

“Th1-like immune response” refers to any adaptive immune response or aspect thereof that is characterized by production of a type 1 interferon, interferon gamma (IFN-gamma), IFN-gamma-inducible 10 kDa protein (IP-10), interleukin 10 (IL-10), interleukin 23 (IL-23), interleukin 12 (IL-12), IgG2a (in mice), IgG1 (in humans), or cell-mediated immunity, or any combination thereof. A Th1-like immune response includes, but is not limited to, a Th1 immune response.

“Th2-like immune response” refers to any adaptive immune response or aspect thereof that is characterized by production of interleukin 4 (IL-4), IgE, IgG1 (in mice), IgG2 (in humans), or humoral immunity, or any combination thereof. A Th2-like immune response includes, but is not limited to, a Th2 immune response.

“Proinflammatory cytokine” refers to any cytokine that is produced or secreted during a proinflammatory immune response. Non-limiting examples of proinflammatory cytokines include TNF-α, IL-6, and IL-1.

“Vaccine” means a composition of matter that improves the immune response to a particular pathogen or disease. A vaccine typically contains factors that stimulate a subject's immune system to recognize a specific antigen as foreign and eliminate it from the subject's body. A vaccine also establishes an immunologic ‘memory’ so the antigen will be quickly recognized and responded to if a person is re-challenged. Vaccines can be prophylactic or therapeutic. In some embodiments, a vaccine can include dosage forms as described herein.

“Positive-sense” as used herein to describe an RNA virus, means a virus that is naturally packaged into viral particles that contain a single strand of RNA that can be directly translated into a protein (i.e., contains a nucleic acid sequence encoding one or more viral proteins).

“Negative-sense” as used herein to describe an RNA virus, means a virus that is naturally packaged into viral particles that contain a single strand of RNA that contains a nucleic acid that is complementary to a nucleic acid sequence that can be directly translated into a protein (i.e., contains an antisense nucleic acid sequence).

By the term “tumor-associated antigen” is meant an antigen that is uniquely expressed or shows an increased level of expression in a cancer cell relative to other cells in the subject (e.g., relative to the non-cancer (normal) cells from the same tissue in the subject). A wide variety of tumor-associated antigens are known in the art. Non-limiting examples of tumor-associated antigens are described herein.

By the term “mixing” is meant placing at least two (e.g., at least three, four, five, or six) different agents together into a single composition. In non-limiting examples, at least one nucleic acid, and at least one carrier, at least one pharmaceutically acceptable excipients, and/or one antigen are placed together to form a single composition.

By the term “reducing the risk of developing a disease” is meant reducing the likelihood that a subject (e.g., a human) will develop the disease as compared to a control population (e.g., a population that receives an alternate treatment or no treatment). Methods for reducing the likelihood that a subject will develop a cancer are described herein. In some embodiments, the subject may be previously identified as having an increased risk of developing a disorder (e.g., a cancer).

As used in this specification and the appended claims, the singular forms “a,” “an,” and “the” include plural referents unless the content clearly dictates otherwise. For example, reference to “a polymer” includes a mixture of two or more such molecules or a mixture of differing molecular weights of a single polymer species, reference to “a solvent” includes a mixture of two or more such solvents, reference to “an adhesive” includes mixtures of two or more such materials, and the like.

### Nucleic Acids

The nucleic acids disclosed herein can have nucleotide sequences obtained or derived from a 5′- or 3′-terminal region of a positive-sense, single-stranded RNA virus genome or from a 5′-terminal region of a negative-sense, single-stranded RNA virus genome. RNA viruses are viruses that have ribonucleic acid as their genetic material. RNA viruses can be further classified according to the sense or polarity of their RNA into negative-sense and positive-sense RNA viruses. Positive-sense viral RNA is similar to mRNA, and thus can be immediately translated by the host cell. Negative-sense viral RNA is complementary to mRNA, and thus must be converted to positive-sense RNA by an RNA polymerase before translation. Positive-sense, single-stranded RNA viruses are classified in Baltimore Group IV, whereas negative-sense single stranded RNA viruses are classified in Baltimore Group V.

The nucleic acids disclosed herein can have nucleotide sequences obtained or derived from any positive-sense or negative-sense single-stranded RNA virus genome. Hundreds of single-stranded RNA virus genomes have been sequenced and their sequences made available, e.g., through the GenBank sequence database. The sequences and other information associated with the GenBank accession numbers listed herein are incorporated by reference.

The positive-sense, single-stranded RNA virus can be a member of the family Flaviviridae. Sequenced Flaviviridae genomes include: NC_{—}012932 (Aedes flavivirus), NC_{—}009029 (Kokobera virus), NC_{—}009028 (Ilheus virus), NC_{—}012812 (Bovine viral diarrhea virus 3 Th/04 KhonKaen), NC_{—}012735 (Wesselsbron virus), NC_{—}006947 (Karshi virus), NC_{—}006551 (Usutu virus), NC_{—}005062 (Omsk hemorrhagic fever virus), NC_{—}005064 (Kamiti River virus), NC_{—}003690 (Langat virus), NC_{—}009026 (Bussuquara virus), NC_{—}008604 (Culex flavivirus), NC_{—}004102 (Hepatitis C virus genotype 1), NC_{—}012671 (Quang Binh virus), NC_{—}004119 (Montana myotis leukoencephalitis virus), NC_{—}003635 (Modoc virus), NC_{—}003675 (Rio Bravo virus), NC_{—}005039 (Yokose virus), NC_{—}003678 (Pestivirus Giraffe-1), NC_{—}003676 (Apoi virus), NC_{—}001809 (Louping ill virus), NC_{—}001564 (Cell fusing agent virus), NC_{—}012534 (Bagaza virus), NC_{—}012533 (Kedougou virus), NC_{—}012532 (Zika virus), NC_{—}001474 (Dengue virus type 2), NC_{—}009942 (West Nile virus (lineage I strain NY99)), NC_{—}009827 (Hepattis C virus genotype 6), NC_{—}009826 (Hepatitis C virus genotype 5), NC_{—}009825 (Hepatitis C virus genotype 4), NC_{—}009824 (Hepatitis C virus genotype 3), NC_{—}009823 (Hepatitis C virus genotype 2), NC_{—}008719 (Sepik virus), NC_{—}008718 (Entebbe bat virus), NC_{—}007580 (St. Louis encephalitis virus), NC_{—}004355 (Alkhurma virus), NC_{—}003996 (Tamana bat virus), NC_{—}003687 (Powassan virus), NC_{—}003679 (Border disease virus X818), NC_{—}002657 (Classical swine fever virus), NC_{—}002640 (Dengue virus type 4), NC_{—}000943 (Murray Valley encephalitis virus), NC_{—}001837 (Hepatitis GB virus A), NC_{—}002032 (Bovine viral diarrhea virus genotype 2), NC_{—}001710 (GB virus C/Hepatitis G virus), NC_{—}001672 (Tick-borne encephalitis virus), NC_{—}001655 (Hepatitis GB virus B), NC_{—}002031 (Yellow fever virus), NC_{—}001563 (West Nile virus (lineage II strain 956)), NC_{—}001477 (Dengue virus type 1), NC_{—}001475 (Dengue virus type 3), NC_{—}001461 (Bovine viral diarrhea virus 1), and NC_{—}001437 (Japanese encephalitis virus).

In some embodiments, the positive-sense single-stranded RNA virus is a member of the family Picornaviridae. Sequenced Picornaviridae genomes include: NC_{—}014336 (Human enterovirus 109), NC_{—}014413 (Turdivirus 3), NC_{—}014412 (Turdivirus 2), NC_{—}014411 (Turdivirus 1), NC_{—}012957 (Salivirus NG-J1), NC_{—}001479 (Encephalomyocarditis virus), NC_{—}001366 (Theilovirus), NC_{—}003077 (Equine rhinitis B virus 2), NC_{—}013115 (Human enterovirus 107), NC_{—}013114 (Human enterovirus 98), NC_{—}013695 (Simian picornavirus strain N2O3), NC_{—}010384 (Simian picornavirus strain N125), NC_{—}011829 (Porcine kobuvirus swine/S-1-HUN/2007/Hungary), NC_{—}013755 (Kobuvirus pig/JY-2010a/CHN), NC_{—}012986 (Human klassevirus 1), NC_{—}010354 (Bovine rhinitis B virus), NC_{—}008250 (Duck hepatitis A virus), NC_{—}006553 (Avian sapelovirus), NC_{—}011452 (Foot-and-mouth disease virus—type SAT 3), NC_{—}011451 (Foot-and-mouth disease virus—type SAT 1), NC_{—}011450 (Foot-and-mouth disease virus—type A), NC_{—}003992 (Foot-and-mouth disease virus—type SAT 2), NC_{—}004915 (Foot-and-mouth disease virus—type Asia 1), NC_{—}004451 (Simian picornavirus 1), NC_{—}004004 (Foot-and-mouth disease virus—type O), NC_{—}003987 (Porcine enterovirus 8), NC_{—}002554 (Foot-and-mouth disease virus—type C), NC_{—}001490 (Human rhinovirus 14, complete genome), NC_{—}012802 (Human cosavirus D1), NC_{—}012801 (Human cosavirus B1), NC_{—}012800 (Human cosavirus A1), NC_{—}012798 (Human cosavirus E1), NC_{—}010411 (Simian picornavirus 17), NC_{—}010415 (Simian enterovirus SV6), NC_{—}010413 (Simian enterovirus SV43), NC_{—}010412 (Simian enterovirus SV19), NC_{—}009891 (Seal picornavirus type 1), NC_{—}009448 (Saffold virus), NC_{—}008715 (Possum enterovirus W6), NC_{—}008714 (Possum enterovirus W1), NC_{—}004421 (Bovine kobuvirus), NC_{—}003988 (Simian enterovirus A), NC_{—}003983 (Equine rhinitis B virus 1), NC_{—}009996 (Human rhinovirus C), NC_{—}009887 (Human enterovirus 100), NC_{—}009750 (Duck hepatitis virus AP), NC_{—}004441 (Porcine enterovirus B), NC_{—}003990 (Avian encephalomyelitis virus), NC_{—}003985 (Porcine teschovirus 1), NC_{—}003982 (Equine rhinitis A virus), NC_{—}003976 (Ljungan virus), NC_{—}001918 (Aichi virus), NC_{—}001897 (Human parechovirus), NC_{—}001859 (Bovine enterovirus), NC_{—}001617 (Human rhinovirus 89), NC_{—}001612 (Human enterovirus A), NC_{—}002058 (Poliovirus), NC_{—}001489 (Hepatitis A virus), NC_{—}001472 (Human enterovirus B), NC_{—}001430 (Human enterovirus D), NC_{—}001428 (Human enterovirus C), NC_{—}010810 (Human TMEV-like cardiovirus), and NC_{—}011349 (Seneca valley virus).

In some embodiments, the positive-sense single-stranded RNA virus is a member of the family Caliciviridae. Sequenced Caliciviridae genomes include: NC_{—}013287 (Calicivirus isolate Allston 2008/US), NC_{—}013286 (Calicivirus isolate Allston 2009/US), NC_{—}012699 (Calicivirus pig/AB90/CAN), NC_{—}004064 (Calicivirus strain NB), NC_{—}000940 (Porcine enteric sapovirus), NC_{—}002615 (European brown hare syndrome virus), NC_{—}008580 (Rabbit vesivirus), NC_{—}006875 (Calicivirus isolate TCG), NC_{—}004542 (Canine calicivirus), NC_{—}011050 (Steller sea lion vesivirus), NC_{—}011704 (Rabbit calicivirus Australia 1 MIC-07), NC_{—}008311 (Murine norovirus 1), NC_{—}006554 (Sapovirus C12 strain C12), NC_{—}006269 (Sapovirus Hu/Dresden/pJG-Sap01/DE), NC_{—}010624 (Sapovirus Mc10), NC_{—}002551 (Vesicular exanthema of swine virus), NC_{—}001959 (Norwalk virus), NC_{—}001543 (Rabbit hemorrhagic disease virus-FRG), NC_{—}001481 (Feline calicivirus), NC_{—}004541 (Walrus calicivirus), and NC_{—}007916 (Newbury agent 1 virus).

In some embodiments, the positive-sense single-stranded RNA virus is a member of the family Togaviridae. Sequenced Togaviridae genomes include: NC_{—}004162 (Chikungunya virus), NC_{—}013528 (Fort Morgan virus), NC_{—}006558 (Getah virus), NC_{—}001547 (Sindbis virus), NC_{—}001544 (Ross River virus), NC_{—}001512 (O′ nyong-nyong virus), NC_{—}012561 (Highlands J virus), NC_{—}003900 (Aura virus), NC_{—}003433 (Sleeping disease virus), NC_{—}003930 (Salmon pancreas disease virus), NC_{—}003908 (Western equine encephalomyelitis virus), NC_{—}001786 (Barmah Forest virus), NC_{—}003899 (Eastern equine encephalitis virus), NC_{—}003417 (Mayaro virus), NC_{—}003215 (Semliki forest virus), NC_{—}001545 (Rubella virus), and NC_{—}001449 (Venezuelan equine encephalitis virus).

In some embodiments, the positive-sense single-stranded RNA virus is a member of the family Coronaviridae. Sequenced Coronaviridae genomes include: NC_{—}004718 (SARS coronavirus), NC_{—}005831 (Human coronavirus L63), NC_{—}014470 (Bat coronavirus BM48-31/BGR/2008), NC_{—}012952 (Feline coronavirus UU8), NC_{—}012938 (Feline coronavirus UU7), NC_{—}012956 (Feline coronavirus UU9), NC_{—}012955 (Feline coronavirus UU10), NC_{—}012954 (Feline coronavirus UU16), NC_{—}012953 (Feline coronavirus UU15), NC_{—}012951 (Feline coronavirus UU11), NC_{—}012950 (Human enteric coronavirus strain 4408), NC_{—}012949 (Bovine respiratory coronavirus bovine/US/OH-440-TC/1996), NC_{—}012948 (Bovine respiratory coronavirus AH187, complete genome), NC_{—}012942 (Feline coronavirus UU3), NC_{—}012941 (Feline coronavirus UU2), NC_{—}012940 (Feline coronavirus UU5), NC_{—}012939 (Feline coronavirus UU4), NC_{—}012937 (Feline coronavirus RM), NC_{—}012936 (Rat coronavirus Parker), NC_{—}002645 (Human coronavirus 229E), NC_{—}013795 (Feline coronavirus UU23), NC_{—}013794 (Feline coronavirus UU22), NC_{—}010646 (Beluga Whale coronavirus SW1), NC_{—}009657 (Scotophilus bat coronavirus 512), NC_{—}007732 (Porcine hemagglutinating encephalomyelitis virus), NC_{—}007025 (Feline coronavirus), NC_{—}007447 (Breda virus), NC_{—}005147 (Human coronavirus OC43), NC_{—}002306 (Transmissible gastroenteritis virus), NC_{—}010438 (Bat coronavirus HKU8), NC_{—}010437 (Bat coronavirus 1A), NC_{—}010436 (Bat coronavirus 1B), NC_{—}010327 (Equine coronavirus), NC_{—}008516 (White bream virus), NC_{—}001846 (Murine hepatitis virus strain A59), NC_{—}001451 (Avian infectious bronchitis virus), NC_{—}011550 (Munia coronavirus HKU13-3514), NC_{—}011549 (Thrush coronavirus HKU12-600), NC_{—}010800 (Turkey coronavirus), NC_{—}009988 (Bat coronavirus HKU2), NC_{—}009021 (Bat coronavirus HKU9-1), NC_{—}009020 (Bat coronavirus HKU5-1), NC_{—}009019 (Bat coronavirus HKU4-1), NC_{—}006852 (Murine hepatitis virus strain JHM), NC_{—}006577 (Human coronavirus HKU1), NC_{—}003436 (Porcine epidemic diarrhea virus), NC_{—}003045 (Bovine coronavirus), NC_{—}008315 (Bat coronavirus (BtCoV/133/2005)).

In some embodiments, the positive-sense single-stranded RNA virus is a member of the family Astroviridae. Sequenced Astroviridae genomes include: NC_{—}005790 (Turkey astrovirus 2), NC_{—}013443 (HMO Astrovirus A), NC_{—}014320 (Astrovirus MLB1 HK05), NC_{—}013060 (Astrovirus VA1), NC_{—}002469 (Ovine astrovirus), NC_{—}003790 (Chicken astrovirus), NC_{—}004579 (Mink astrovirus), NC_{—}002470 (Turkey astrovirus), NC_{—}012437 (Duck astrovirus C-NGB), NC_{—}001943 (Human astrovirus), and NC_{—}011400 (Astrovirus MLB1).

In some embodiments, the negative-sense single-stranded RNA virus is a member of the family Filoviridae. Sequenced Filoviridae genomes include: NC_{—}014372 (Cote d'Ivoire ebolavirus), NC_{—}004161 (Reston ebolavirus), NC_{—}002549 (Zaire ebolavirus), NC_{—}014373 (Bundibugyo ebolavirus), NC_{—}006432 (Sudan ebolavirus), and NC_{—}001608 (Lake Victoria marburgvirus—Musoke).

In some embodiments, the negative-sense single-stranded RNA virus is a member of the family Arenaviridae. Sequenced Arenaviridae genomes include: NC_{—}012777 (Lujo virus segment L), NC_{—}012776 (Lujo virus segment S), NC_{—}013058 (Morogoro virus segment L), NC_{—}013057 (Morogoro virus segment S), NC_{—}006575 (Mopeia virus AN20410 segment S), NC_{—}006574 (Mopeia virus AN20410 segment L), NC_{—}006573 (Mopeia Lassa reassortant 29 segment S), NC_{—}006572 (Mopeia Lassa reassortant 29 segment L), NC_{—}006313 (Sabia virus segment L), NC_{—}005897 (Pirital virus segment L), NC_{—}005082 (Guanarito virus segment L), NC_{—}005081 (Junin virus segment S), NC_{—}005080 (Junin virus segment L), NC_{—}005079 (Machupo virus segment L), NC_{—}010756 (Parana virus segment S (small)), NC_{—}010701 (Tamiami virus segment S (small)), NC_{—}006447 (Pichinde virus), NC_{—}006439 (Pichinde virus L RNA), NC_{—}006317 (Sabia virus), NC_{—}005078 (Machupo virus segment S), NC_{—}005077 (Guanarito virus segment S), NC_{—}010252 (Cupixi virus segment L), NC_{—}010250 (Oliveros virus segment L), NC_{—}010249 (Allpahuayo virus segment L), NC_{—}010248 (Oliveros virus segment S), NC_{—}004293 (Tacaribe virus segment S), NC_{—}004292 (Tacaribe virus segment L), NC_{—}010758 (Latino virus segment S), NC_{—}010757 (Flexal virus segment S), NC_{—}010703 (Whitewater Arroyo virus segment L), NC_{—}010702 (Tamiami virus segment L), NC_{—}010700 (Whitewater Arroyo virus segment S), NC_{—}010256 (Bear Canyon virus segment S), NC_{—}010255 (Bear Canyon virus segment L), NC_{—}010253 (Allpahuayo virus segment S), NC_{—}010251 (Amapari virus segment L), NC_{—}010247 (Amapari virus segment S), NC_{—}005894 (Pirital virus segment S), NC_{—}004297 (Lassa virus segment L), NC_{—}004296 (Lassa virus segment S), NC_{—}004294 (Lymphocytic choriomeningitis virus segment S), NC_{—}004291 (Lymphocytic choriomeningitis virus segment L), NC_{—}010761 (Parana virus segment L), NC_{—}010760 (Latino virus segment L), NC_{—}010759 (Flexal virus segment L), NC_{—}010254 (Cupixi virus segment S), NC_{—}007906 (Ippy virus segment L), NC_{—}007905 (Ippy virus segment S), NC_{—}007904 (Mobala virus segment L), NC_{—}007903 (Mobala virus segment S), NC_{—}010563 (Chapare virus segment L), and NC_{—}010562 (Chapare virus segment S).

In some embodiments, the negative-sense single-stranded RNA virus is a member of the family Bunyaviridae. Sequenced Bunyaviridae genomes include: NC_{—}003468 (Andes virus segment L), NC_{—}003467 (Andes virus segment M), NC_{—}005219 (Hantaan virus), NC_{—}006435 (Hantavirus Z10 chromosome L), NC_{—}006433 (Hantavirus Z10 chromosome S segment), NC_{—}006437 (Hantavirus Z10 segment M), NC_{—}005218 (Hantaan virus), NC_{—}005217 (Sin Nombre virus chromosome L segment), NC_{—}005216 (Sin Nombre virus chromosome S segment), NC_{—}005215 (Sin Nombre virus chromosome M segment), NC_{—}005225 (Puumala virus segment L), NC_{—}010704 (Thottapalayam virus segment S), NC_{—}005226 (Tula virus segment L), NC_{—}005228 (Tula virus segment M), NC_{—}010708 (Thottapalayam virus segment M), NC_{—}010707 (Thottapalayam virus segment L), NC_{—}005238 (Seoul virus strain Seoul 80-39 clone 1), NC_{—}005237 (Seoul virus segment M), NC_{—}005236 (Seoul virus strain 80-39 segment S), NC_{—}005235 (Dobrava-Belgrade virus strain DOBV/Ano-Poroia/Afl9/1999), NC_{—}005234 (Dobrava virus segment M), NC_{—}005233 (Dobrava virus segment S), NC_{—}005227 (Tula virus segment S), NC_{—}005224 (Puumala virus segment S), NC_{—}005223 (Puumala virus segment M), NC_{—}005222 (Hantaan virus segment L), NC_{—}003466 (Andes virus segment S), NC_{—}004159 (Dugbe virus segment L), NC_{—}004158 (Dugbe virus segment M), NC_{—}005302 (Crimean-Congo hemorrhagic fever virus segment S), NC_{—}005301 (Crimean-Congo hemorrhagic fever virus segment L), NC_{—}005300 (Crimean-Congo hemorrhagic fever virus segment M), NC_{—}004157 (Dugbe virus segment S), NC_{—}001927 (Bunyamwera virus segment S), NC_{—}005777 (Oropouche virus segment S), NC_{—}005776 (Oropouche virus segment L), NC_{—}004110 (La Crosse virus segment S), NC_{—}004109 (La Crosse virus segment M), NC_{—}004108 (La Crosse virus segment L), NC_{—}009894 (Akabane virus segment L), NC_{—}001925 (Bunyamwera virus L segment), NC_{—}009896 (Akabane virus segment S), NC_{—}009895 (Akabane virus segment M), NC_{—}005775 (Oropouche virus segment M), NC_{—}001926 (Bunyamwera virus M segment), NC_{—}014397 (Rift Valley fever virus segment L), NC_{—}014396 (Rift Valley fever virus segment M), NC_{—}014395 (Rift Valley fever virus segment S), NC_{—}005221 (Uukuniemi virus), NC_{—}005220 (Uukuniemi virus chromosome segment M), NC_{—}005214 (Uukuniemi virus segment L), NC_{—}006320 (Toscana virus segment M), NC_{—}006319 (Toscana virus segment L), and NC_{—}006318 (Toscana virus segment S).

In some embodiments, the negative-sense single-stranded RNA virus is a member of the family Bornaviridae. One exemplary sequenced Bornaviridae genome is NC_{—}001607 (Boma disease virus).

In some embodiments, the negative-sense single-stranded RNA virus is a member of the family Paramyxoviridae. Sequenced Paramyxoviridae genomes include: NC_{—}001498 (Measles virus), NC_{—}002200 (Mumps virus), NC_{—}005339 (Mossman virus), NC_{—}002199 (Tupaia paramyxovirus), NC_{—}001552 (Sendai virus), NC_{—}006430 (Parainfluenza virus 5), NC_{—}006579 (Pneumonia virus of mice J3666), NC_{—}003043 (Avian paramyxovirus 6), NC_{—}007620 (Menangle virus), NC_{—}007454 (J-virus), NC_{—}005036 (Goose paramyxovirus SF02), NC_{—}004074 (Tioman virus), NC_{—}002617 (Newcastle disease virus B1), NC_{—}001921 (Canine distemper virus), NC_{—}001906 (Hendra virus), NC_{—}001781 (Human respiratory syncytial virus), NC_{—}001989 (Bovine respiratory syncytial virus), NC_{—}001803 (Respiratory syncytial virus), NC_{—}003461 (Human parainfluenza virus 1), NC_{—}009640 (Porcine rubulavirus), NC_{—}007803 (Beilong virus), NC_{—}006428 (Simian virus 41), NC_{—}005283 (Dolphin morbillivirus), NC_{—}005084 (Fer-de-lance virus), NC_{—}003443 (Human parainfluenza virus 2), NC_{—}001796 (Human parainfluenza virus 3), NC_{—}007652 (Avian metapneumovirus), NC_{—}006383 (Peste-des-petits-ruminants virus), NC_{—}006296 (Rinderpest virus (strain Kabete O)), NC_{—}004148 (Human metapneumovirus), NC_{—}002728 (Nipah virus), NC_{—}002161 (Bovine parainfluenza virus 3), and NC_{—}009489 (Mapuera virus).

In some embodiments, the negative-sense single-stranded RNA virus is a member of the family Rhabdoviridae. Sequenced Rhabdoviridae genomes include: NC_{—}003243 (Australian bat lyssavirus), NC_{—}009528 (European bat lyssavirus 2), NC_{—}009527 (European bat lyssavirus 1), NC_{—}006429 (Mokola virus), NC_{—}001542 (Rabies virus), NC_{—}002803 (Spring viraemia of carp virus), NC_{—}001560 (Vesicular stomatitis Indiana virus), NC_{—}002526 (Bovine ephemeral fever virus), NC_{—}005093 (Hirame rhabdovirus), NC_{—}000903 (Snakehead rhabdovirus), NC_{—}001652 (Infectious hematopoietic necrosis virus), NC_{—}000855 (Viral hemorrhagic septicemia virus), NC_{—}013955 (Ngaingan virus), NC_{—}007020 (Tupaia virus), NC_{—}011639 (Wongabel virus).

In some embodiments, the negative-sense single-stranded RNA virus is a member of the family Orthomyxoviridae. Sequenced Orthomyxoviridae genomes include: NC_{—}007364 (Influenza A virus (A/Goose/Guangdong/1/96(H5N1)), segment 8), NC_{—}007363 (Influenza A virus (A/Goose/Guangdong/1/96(H5N1)) strain A/Goose/Guangdong/1/96(H5N1), segment 7), NC_{—}007378 (Influenza A virus (A/Korea/426/1968(H2N2)), segment 1), NC_{—}007361 (Influenza A virus (A/Goose/Guangdong/1/96(H5N1)) strain A/Goose/Guangdong/1/96(H5N1), segment 6), NC_{—}007360 (Influenza A virus (A/Goose/Guangdong/1/96(H5N1)), segment 5), NC_{—}007359 (Influenza A virus (A/Goose/Guangdong/1/96(H5N1)), segment 3), NC_{—}007357 (Influenza A virus (A/Goose/Guangdong/1/96(H5N1)), segment 1), NC_{—}007377 (Influenza A virus (A/Korea/426/68(H2N2)) segment 7), NC_{—}007376 (Influenza A virus (A/Korea/426/68(H2N2)) segment 3), NC_{—}007374 (Influenza A virus (A/Korea/426/68(H2N2)) segment 4), NC_{—}007373 (Influenza A virus (A/New York/392/2004(H3N2)) segment 1), NC_{—}007372 (Influenza A virus (A/New York/392/2004(H3N2)) segment 2), NC_{—}007371 (Influenza A virus (A/New York/392/2004(H3N2)) segment 3), NC_{—}007370 (Influenza A virus (A/New York/392/2004(H3N2)) segment 8), NC_{—}007369 (Influenza A virus (A/New York/392/2004(H3N2)) segment 5), NC_{—}007368 (Influenza A virus (A/New York/392/2004(H3N2)) segment 6), NC_{—}007367 (Influenza A virus (A/New York/392/2004(H3N2)) segment 7), NC_{—}007366 (Influenza A virus (A/New York/392/2004(H3N2)) segment 4), NC_{—}007362 (Influenza A virus (A/Goose/Guangdong/1/96(H5N1)) segment 4), NC_{—}007358 (Influenza A virus (A/Goose/Guangdong/1/96(H5N1)) segment 2), NC_{—}004912 (Influenza A virus (A/Hong Kong/1073/99(H9N2)) segment 3), NC_{—}004911 (Influenza A virus (A/Hong Kong/1073/99(H9N2)) segment 2), NC_{—}004910 (Influenza A virus (A/Hong Kong/1073/99(H9N2)) segment 1), NC_{—}004909 (Influenza A virus (A/Hong Kong/1073/99(H9N2)) segment 6), NC_{—}004908 (Influenza A virus (A/Hong Kong/1073/99(H9N2)) segment 4), NC_{—}004907 (Influenza A virus (A/Hong Kong/1073/99(H9N2)) segment 7), NC_{—}004906 (Influenza A virus (A/Hong Kong/1073/99(H9N2)) segment 8), NC_{—}004905 (Influenza A virus (A/Hong Kong/1073/99(H9N2)) segment 5), NC_{—}002023 (Influenza A virus (A/Puerto Rico/8/34(H1N1)) segment 1), NC_{—}002022 (Influenza A virus (A/Puerto Rico/8/34(H1N1)) segment 3), NC_{—}002020 (Influenza A virus (A/Puerto Rico/8/34(H1N1)) segment 8), NC_{—}002018 (Influenza A virus (A/Puerto Rico/8/34(H1N1)) segment 6), NC_{—}002017 (Influenza A virus (A/Puerto Rico/8/34(H1N1)) segment 4), NC_{—}002016 (Influenza A virus (A/Puerto Rico/8/34(H1N1)) segment 7), NC_{—}007375 (Influenza A virus (A/Korea/426/68(H2N2)) segment 2), NC_{—}002021 (Influenza A virus (A/Puerto Rico/8/34(H1N1)) segment 2), NC_{—}002019 (Influenza A virus (A/Puerto Rico/8/34(H1N1)) segment 5), NC_{—}007382 (Influenza A virus (A/Korea/426/68(H2N2)) segment 6), NC_{—}007381 (Influenza A virus (A/Korea/426/68(H2N2)) segment 5), NC_{—}007380 (Influenza A virus (A/Korea/426/68(H2N2)) segment 8), NC_{—}002211 (Influenza B virus RNA 8), NC_{—}002209 (Influenza B virus RNA 6), NC_{—}002208 (Influenza B virus RNA 5), NC_{—}002206 (Influenza B virus RNA-3), NC_{—}002205 (Influenza B virus RNA-2), NC_{—}002204 (Influenza B virus RNA 1), NC_{—}002210 (Influenza B virus RNA 7), NC_{—}002207 (Influenza B virus RNA 4), NC_{—}006312 (Influenza C virus (C/Ann Arbor/1/50) segment 6), NC_{—}006311 (Influenza C virus (C/Ann Arbor/1/50) segment 5), NC_{—}006310 (Influenza C virus (C/Ann Arbor/1/50) segment 4), NC_{—}006309 (Influenza C virus (C/Ann Arbor/1/50) segment 3), NC_{—}006308 (Influenza C virus (C/Ann Arbor/1/50) segment 2), NC_{—}006307 (Influenza C virus (C/Ann Arbor/1/50) segment 1), NC_{—}006306 (Influenza C virus (C/Ann Arbor/1/50) segment 7), NC_{—}006496 (Thogoto virus chromosome segment 3), NC_{—}006495 (Thogoto virus chromosome segment 2), NC_{—}006506 (Thogoto virus segment 4), NC_{—}006504 (Thogoto virus segment 6), NC_{—}006508 (Thogoto virus segment 1), and NC_{—}006507 (Thogoto virus segment 5).

In some embodiments, this disclosure includes nucleic acid sequences that are substantially identical to a nucleotide sequence obtained or derived from a 5′- or 3′-terminal region of a positive-sense, single-stranded RNA virus genome or from a 5′-terminal region of a negative-sense, single-stranded RNA virus genome. A nucleic acid sequence that is “substantially identical” to such a nucleic acid is at least 75% identical (e.g., at least about 80%, 85%, 90%, 95, 98%, or 99% identical) to the nucleic acid sequence.

To determine the percent identity of two amino acid or nucleic acid sequences, the sequences are aligned for optimal comparison purposes (i.e., gaps can be introduced as required in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of overlapping positions×100). The two sequences can be of the same length.

The percent identity or homology between two sequences can be determined using a mathematical algorithm. For the purposes of our definition, the percent identity or homology between two sequences is determined using the algorithm of Karlin and Altschul, 1990, Proc. Natl. Acad. Sci. U.S.A., 87:2264-68, as modified in Karlin and Altschul, 1993, Proc. Natl. Acad. Sci. USA, 90:5873-77. This algorithm is incorporated into the NBLAST program of Altschul et al., 1990, J. Mol. Biol., 215:403-410. To obtain gapped alignments for comparison purposes, Gapped BLAST can be utilized as described in Altschul et al., 1997, Nucleic Acids Res., 25:3389-3402.

The compositions described herein can include certain artificially synthesized oligonucleotides having a base sequence that corresponds to a base sequence found in nature, i.e., a base sequence found in the 3′ end of a single-stranded minus-sense RNA virus genome. The compositions are artificially synthesized to include the feature of the stabilized backbone. The backbone of an oligonucleotide can be stabilized using any suitable chemical method or modification, provided the oligonucleotide having a stabilized backbone is relatively more resistant to nuclease degradation than a corresponding oligonucleotide having an all-phosphodiester backbone.

The immunostimulatory oligonucleotides described herein can encompass various chemical modifications and substitutions, in comparison to natural RNA and DNA, involving a phosphodiester internucleoside bridge, a beta-D-ribose unit, and/or a natural nucleoside base (adenine, guanine, cytosine, thymine, and uracil). Examples of chemical modifications are known to the skilled person and are described, for example, in Uhlmann et al., 1990, Chem. Rev. 90:543; “Protocols for Oligonucleotides and Analogs” in Synthesis and Properties & Synthesis and Analytical Techniques, S. Agrawal, Ed., Humana Press, Totowa, USA 1993; Crooke et al., 1996, Ann. Rev. Pharmacol. Toxicol. 36:107-29; and Hunziker et al., 1995, Mod. Synth. Methods 7:331-417. An oligonucleotide described herein can have one or more modifications, wherein each modification is located at a particular internucleoside bridge and/or at a particular beta-D-ribose unit and/or at a particular natural nucleoside base position in comparison to an oligonucleotide of the same sequence which is composed of natural DNA or RNA.

For example, the oligonucleotides can include one or more modifications wherein each modification is independently selected from:

a) the replacement of a phosphodiester internucleoside bridge located at the 3′ and/or the 5′ end of a nucleoside by a modified internucleoside bridge;

b) the replacement of a phosphodiester internucleoside bridge located at the 3′ and/or the 5′ end of a nucleoside by a dephospho bridge;

c) the replacement of a sugar phosphate unit from the sugar phosphate backbone by another unit;

d) the replacement of a beta-D-ribose unit by a modified sugar unit; and

e) the replacement of a natural nucleoside base by a modified nucleoside base.

More detailed examples for the chemical modification of an oligonucleotide are as follows.

The oligonucleotides can include modified internucleoside linkages, such as those described in a) or b) above. These modified linkages can be partially resistant to degradation (e.g., are stabilized). A “stabilized oligonucleotide molecule” is an oligonucleotide molecule that is relatively resistant to in vivo degradation (e.g., via an exo- or endo-nuclease) resulting from such modifications. Oligonucleotides having phosphorothioate linkages, in some embodiments, can provide maximal activity and protect the oligonucleotide from degradation by intracellular exo- and endo-nucleases. A phosphodiester internucleoside bridge located at the 3′ and/or the 5′ end of a nucleoside can be replaced by a modified internucleoside bridge, wherein the modified internucleoside bridge is, for example, selected from phosphorothioate, phosphorodithioate, NR¹R²-phosphoramidate, boranophosphate, alpha-hydroxybenzyl phosphonate, phosphate-(C1-C21)-O-alkyl ester, phosphate-[(C6-C2)aryl-(C1-C21)-β-alkyl]ester, (C1-C8)alkylphosphonate and/or (C6-C12)arylphosphonat-e bridges, (C7-C12)-alpha-hydroxymethyl-aryl (e.g., disclosed in WO 95/01363), wherein (C6-C12)aryl, (C6-C20)aryl, and (C6-C14)aryl are optionally substituted by halogen, alkyl, alkoxy, nitro, cyano, and where R¹ and R² are, independently of each other, hydrogen, (C1-C8)-alkyl, (C6-C20)-aryl, (C6-C14)-aryl-(C1-C8)-alkyl, e.g., hydrogen, (C1-C8)-alkyl, (C1-C4)-alkyl and/or methoxyethyl, or R¹ and R² form, together with the nitrogen atom carrying them, a 5-6-membered heterocyclic ring which can additionally contain a further heteroatom from the group O, S, and N.

The replacement of a phosphodiester bridge located at the 3′ and/or the 5′ end of a nucleoside by a dephospho bridge (dephospho bridges are described, for example, in Uhlmann and Peyman, “Methods in Molecular Biology”, Vol. 20, “Protocols for Oligonucleotides and Analogs”, S. Agrawal, Ed., Humana Press, Totowa 1993, Chapter 16, pp. 355 ff), e.g., can be a dephospho bridge selected from the dephospho bridges formacetal, 3′-thioformacetal, methylhydroxylamine, oxime, methylenedimethyl-hydrazo, dimethylenesulfone, and/or silyl groups.

A sugar phosphate unit (i.e., a beta-D-ribose and phosphodiester internucleoside bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al. (1989) Nucleic Acids Res 17:6129-41), that is, e.g., the replacement by a morpholino-derivative unit; or to build up a polyamide nucleic acid (“PNA;” as described for example, in Nielsen et al., 1994, Bioconjug. Chem. 5:3-7), that is, e.g., the replacement by a PNA backbone unit, e.g., by 2-aminoethylglycine. The oligonucleotide can have other carbohydrate backbone modifications and replacements, such as peptide nucleic acids with phosphate groups (PHONA), locked nucleic acids (LNA), and oligonucleotides having backbone sections with alkyl linkers or amino linkers. The alkyl linker can be branched or unbranched, substituted or unsubstituted, and chirally pure or a racemic mixture.

In addition to the stabilized backbones disclosed above, the compositions described herein can alternatively or in addition contain pyrophosphate internucleoside linkages. The synthesis and ribonuclease inhibition by 3′,5′-pyrophosphate-linked nucleotides have been described, for example, in Russo et al., 1999, J. Biol. Chem., 274:14902-8.

The compositions described herein can alternatively or in addition contain a chimeric RNA:DNA backbone in which at least one nucleotide is a deoxynucleotide, e.g., a deoxyribonucleotide. The number and position of the at least one deoxynucleotide can affect immunostimulatory activity of the oligonucleotide. In various embodiments the number of deoxynucleotides in an immunostimulatory nucleic acid can be 1, 2, 3, 4, 5, 6, 7, 8, 9, 10, 11, 12, 13, 14, 15, 16, 17, 18, 19, 20, 21, 22, 23, 24, 25, or 26. In some embodiments in which there is more than one deoxynucleotide, deoxynucleotides are adjacent (i.e., directly linked) to one another. In various embodiments the number of consecutive adjacent deoxynucleotides can be 2, 3, 4, 5, 6, 7, 8, 9, 10, 11, 12, 13, 14, 15, 16, 17, 18, 19, 20, 21, 22, 23, 24, 25, or 26. Groups of adjacent deoxynucleotides can also be present, separated from one another by at least one intervening nucleotide that is not a deoxynucleotide. In some embodiments in which there is more than one deoxynucleotide, no deoxynucleotide is adjacent to another deoxynucleotide. In some embodiments the position of the at least one deoxynucleotide can increase the immunostimulatory effect of the oligonucleotide compared to a corresponding oligonucleotide that is strictly RNA. In other embodiments the position of the at least one deoxynucleotide can decrease the immunostimulatory effect of the oligonucleotide compared to a corresponding oligonucleotide that is strictly RNA.

Nucleic acid compositions described herein can include modified sugar units. A beta-ribose unit or a beta-D-2′-deoxyribose unit can be replaced by a modified sugar unit, wherein the modified sugar unit is for example selected from beta-D-ribose, alpha-D-2′-deoxyribose, L-2′-deoxyribose, 2′-F-2′-deoxyribose, 2′-F-arabinose, 2′-O—(C1-C6)alkyl-ribose, 2′-O-methylribose, 2′-O—(C2-C6)alkenyl-ribose, 2′-[O—(C1-C6)alkyl-O—(C.-sub.1-C6)alkyl]-ribose, 2′—NH2-2′-deoxyribose, beta-D-xylo-furanose, alpha-arabinofuranose, 2,4-dideoxy-beta-D-erythro-hexo-pyranose, and carbocyclic (described, for example, in Froehler, 1992, J. Am. Chem. Soc. 114:8320) and/or open-chain sugar analogs (described, for example, in Vandendriessche et al., 1993, Tetrahedron 49:7223) and/or bicyclosugar analogs (described, for example, in Tarkov et al., 1993, Helv. Chim Acta 76:481).

Nucleic acid compositions described herein can include nucleosides found in nature, including guanosine, cytidine, adenosine, thymidine, and uridine, but the nucleic acid compositions are not so limited. Nucleic acid compositions described herein can include modified nucleosides. Modified nucleosides include nucleoside derivatives with modifications involving the base, the sugar, or both the base and the sugar.

Nucleic acids also include substituted purines and pyrimidines such as C-5 propyne pyrimidine and 7-deaza-7-substituted purine modified bases (see, e.g., Wagner et al., 1996, Nat. Biotechnol. 14:840-4. Purines and pyrimidines include, but are not limited to, adenine, cytosine, guanine, thymine, and uracil, and other naturally and non-naturally occurring nucleobases, substituted and unsubstituted aromatic moieties.

A modified base is any base which is chemically distinct from the naturally occurring bases typically found in DNA and RNA, such as T, C, G, A, and U, but which shares basic chemical structure with at least one of these naturally occurring bases. The modified nucleoside base can be, for example, selected from hypoxanthine, dihydrouracil, pseudouracil, 2-thiouracil, 4-thiouracil, 5-aminouracil, 5-(C1-C6)-alkyluraci-1,5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-(C2-C6)-alkenylcytosine, 5-(C2-C6)-alkynylcytosine, 5-chlorocytosine, 5-fluorocytosine, 5-bromocytosine, N.sup.2-dimethylguanine, 2,4-diamino-purine, 8-azapurine, a substituted 7-deazapurine (e.g., 7-deaza-7-substituted purine and/or 7-deaza-8-substituted purine), 5-hydroxymethylcytosine, N4-alkylcytosine, e.g., N4-ethylcytosine, 5-hydroxydeoxycytidine, 5-hydroxymethyldeoxycytid-ine, N4-alkyldeoxycytidine, e.g., N4-ethyldeoxycytidine, 6-thiodeoxyguanosine, and deoxyribonucleosides of nitropyrrole, C5-propynylpyrimidine, and diaminopurine e.g., 2,6-diaminopurine, inosine, 5-methylcytosine, 2-aminopurine, and 2-amino-6-chloropurine, or other modifications of a natural nucleoside base. This list is meant to be exemplary and is not to be interpreted to be limiting.

In particular embodiments described herein modified bases can be incorporated. For instance a cytosine can be replaced with a modified cytosine. A modified cytosine as used herein is a naturally occurring or non-naturally occurring pyrimidine base analog of cytosine which can replace this base without impairing the immunostimulatory activity of the oligonucleotide.

Modified cytosines include, but are not limited to, 5-substituted cytosines (e.g., 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g., N4-ethyl-cytosine), 5-aza-cytosine, 2-mercapto-cytosine, isocytosine, pseudo-isocytosine, cytosine analogs with condensed ring systems (e.g., N,N′-propylene cytosine or phenoxazine), and uracil and its derivatives (e.g., 5-fluoro-uracil, 5-bromo-uracil, 5-bromovinyl-uracil, 4-thio-uracil, 5-hydroxy-uracil, 5-propynyl-uracil). In certain some embodiments, the cytosine base is substituted by a universal base (e.g., 3-nitropyrrole, P-base), an aromatic ring system (e.g., fluorobenzene or difluorobenzene), or a hydrogen atom (Spacer or dSpacer).

Cytidine derivatives generally will also include, without limitation, cytidines with modified sugars. Cytidines with modified sugars include but are not limited to cytosine-beta-D-arabinofuranoside (Ara-C), ribo-C, and 2′-O—(C1-C6)alkyl-cytidine (e.g., 2′-O-methylcytidine, 2′-OMe-C).

A guanine can be replaced with a modified guanine base. A modified guanine as used herein is a naturally occurring or non-naturally occurring purine base analog of guanine which can replace this base without impairing the immunostimulatory activity of the oligonucleotide.

Modified guanines include, but are not limited to, 7-deazaguanine, 7-deaza-7-substituted guanine (such as 7-deaza-7-(C2-C6)alkynylguanine), 7-deaza-8-substituted guanine, hypoxanthine, N2-substituted guanines (e.g., N2-methyl-guanine), 5-amino-3-methyl-3H,6H-thiazolo[4,5-d]pyrimidi-ne-2,7-dione, 2,6-diaminopurine, 2-aminopurine, purine, indole, adenine, substituted adenines (e.g., N6-methyl-adenine, 8-oxo-adenine), 8-substituted guanine (e.g., 8-hydroxyguanine and 8-bromoguanine), and 6-thioguanine. In certain embodiments, the guanine base is substituted by a universal base (e.g., 4-methyl-indole, 5-nitro-indole, and K-base), an aromatic ring system (e.g., benzimidazole or dichloro-benzimidazole, 1-methyl-1H-[1,2,4]triazole-3-carboxylic acid amide), or a hydrogen atom (Spacer or dSpacer).

The nucleic acid compositions described herein can include oligonucleotides of 10 to 200 nucleotides long. It is believed, however, that oligonucleotides as short as 4 or 5 nucleotides in length can be sufficient to bind to a TLR. In various embodiments the oligonucleotide is 10, 11, 12, 13, 14, 15, 16, 17, 18, 19, 20, 21, 22, 23, 24, 25, 26, 27, 28, 29, or 30 nucleotides long. In certain embodiments the oligonucleotides are 10 to 20 nucleotides long. In one embodiment, the oligonucleotide is 10 nucleotides long.

The immunostimulatory compositions described herein can contain, e.g., a nucleic acid of 10 to 200 nucleotides (e.g., 10 to 20, 15 to 25, 20 to 30, 25 to 35, 30 to 50, 15 to 150, 20 to 150, 10 to 100, 10 to 150, or 40 to 100 nucleotides), that contains a 4- to 80-nucleotide sequence (e.g., to 4 to 10, 5 to 15, 10 to 20, 15 to 25, 20 to 30, or 20 to 50-nucleotide sequence), from anywhere within the first 80 bases from a 5′- or 3′-terminus of a positive-sense single-stranded RNA virus genome. For example, the sequence of 4 to 80 nucleotides can be derived from a sequence starting at the end nucleotide of the 5′- or 3′-terminus, or may begin at any nucleotide within 76 nucleotides of the end nucleotide of the 5′ or the 3′-terminus of the positive-sense single-stranded RNA virus genome. The additional sequences present in the 10 to 200 nucleotide sequence can be one or more other immunostimulatory nucleic acids or a random nucleic acid sequence as described herein.

The immunostimulatory compositions described herein can contain, e.g., a nucleic acid of 10 to 200 nucleotides (e.g., 10 to 20, 15 to 25, 20 to 30, 25 to 35, 30 to 50, 15 to 150, 20 to 150, 20 to 100, 10 to 150, or 40 to 100 nucleotides), that contains a 4- to 80-nucleotide sequence (e.g., to 4 to 10, 5 to 15, 10 to 20, 15 to 25, 20 to 30, or 20 to 50-nucleotide sequence) from anywhere within the first 80 bases from a 5′-terminus of a negative-sense single-stranded RNA virus genome. For example, the sequence of 4 to 80 nucleotides can be derived from a sequence starting at the end nucleotide of the 5′-terminus, or may begin at any nucleotide within 76 nucleotides of the end nucleotide of the 5′-terminus of the negative-sense single-stranded RNA virus genome. The immunostimulatory compositions described herein can be formed by placing the sequence derived from the 5′- or 3′-terminus of a positive-sense single-stranded RNA virus genome or the 5′-terminus of a negative-sense single-stranded RNA virus genome at the 5′-terminus and/or 3′-terminus of another nucleic acid to yield a nucleic acid having a total of 10 to 200 nucleotides. The immunostimulatory compositions described herein can also be formed by placing the sequence derived from the 5′- or 3′-terminus of a positive-sense single-stranded RNA virus genome or the 5′-terminus of a negative-sense single-stranded RNA virus genome between the 5′-terminus of a first nucleic acid and the 3′-terminus of a second nucleic acid to yield a nucleic acid having a total of 10 to 200 nucleotides.

Non-limiting examples of a 4- to 80-nucleotide sequence from anywhere within the first 80 bases from a 5′- or 3′-terminus of a positive-sense single-stranded RNA virus genome or a 5′-terminus of a negative-sense single-stranded RNA virus genome are described in the Examples or listed in Table 1 (e.g., any one of SEQ ID NOS: 1-19).

In some embodiments, the immunostimulatory composition contains a nucleic acid of 10 to 200 nucleotides that contains two or more (e.g., two, three, four, five, six, or seven) 2- to 80-nucleotide sequences (e.g., 2- to 5-, 2- to 10-, 10- to 15-, or 15- to 20-nucleotide sequence) from anywhere within the first 80 bases from a 5′- or 3′-terminus of a positive-sense single-stranded RNA virus genome, or two or more (e.g., two, three, four, five, six, or seven) 2- to 80-nucleotide sequences (e.g., 2- to 5-, 2- to 10-, 10- to 15-, or 15- to 20-nucleotide sequence) from anywhere within the first 80 bases from a 5′-terminus of a negative-sense single-stranded RNA virus genome. In some embodiments, the nucleic acid of 10 to 200 nucleotides contains two or more 2- to 80-nucleotide sequences from anywhere within the first 80 bases from a 5′- or 3′-terminus of a positive-sense single-stranded RNA virus genome, or two or more (e.g., two, three, four, five, six, or seven) 2- to 80-nucleotide sequences from anywhere within the first 80 bases from a 5′-terminus of a negative-sense single-stranded RNA directly, where two or more (e.g., three, four, five, six, or all) of the individual 2- to 80-nucleotide sequences directly abut each other (e.g., the 5′ end of a first sequence directly abuts the 3′ end of a second sequence).

In some embodiments, the nucleic acid of 10 to 200 nucleotides contains two or more 2- to 80-nucleotide sequences from anywhere within the first 80 bases from a 5′- or 3′-terminus of a positive-sense single-stranded RNA virus genome, or two or more (e.g., two, three, four, five, six, or seven) 2- to 80-nucleotide sequences from anywhere within the first 80 bases from a 5′-terminus of a negative-sense single-stranded RNA, where two or more (e.g., three, four, five, six, or all) of the individual 2- to 80-nucleotide sequences do not directly abut each other (e.g., one or more (e.g., 2-5, 6-10, 11-15, or 16-20) nucleotides (e.g., any of the additional immunostimulatory nucleic acid sequences described herein or known in the art or a random nucleic acid sequence) are present between the 5′ end of a first sequence and the 3′ end of a second 2- to 80-nucleotide sequence). In some embodiments, the different individual 2- to 80-nucleotide sequences are derived from different (non-contiguous) or overlapping sequences present within the first 80 bases from a 5′- or 3′-terminus of a positive-sense single-stranded RNA virus genome, or the different individual 2- to 80-nucleotide sequences are derived from different (non-contiguous) or overlapping sequences within the first 80 bases from a 5′-terminus of a negative-sense single-stranded RNA genome. In some embodiments, the nucleic acid of 10 to 200 nucleotides contains two or more (e.g., two, three, four, five, six, or seven) copies of the same 2- to 80-nucleotide sequence present within the first 80 bases from a 5′- or 3′-terminus of a positive-sense single-stranded RNA virus genome, or contains two or more (e.g., two, three, four, five, six, or seven) copies of the same 2- to 80-nucleotide sequence present within the first 80 bases from a 5′-terminus of a negative-sense single-stranded RNA virus genome.

In some embodiments, the immunostimulatory composition contains a nucleic acid of 10 to 200 nucleotides that contains one or more (e.g., one, two, three, four, five, or six) 2- to 80-nucleotide sequence(s) (e.g., 2- to 5-, 2- to 10-, 10- to 15-, or 15- to 20-nucleotide sequence) from anywhere within the first 80 bases from a 5′- or 3′-terminus of a positive-sense single-stranded RNA virus genome, and one or more (e.g., one, two, three, four, five, or six) 2- to 80-nucleotide sequence(s) (e.g., 2- to 5-, 2- to 10-, 10- to 15-, or 15- to 20-nucleotide sequence) from anywhere within the first 80 bases from a 5′-terminus of a negative-sense single-stranded RNA virus genome. In some embodiments, the nucleic acid of 10 to 200 nucleotides contains one or more 2- to 80-nucleotide sequences from anywhere within the first 80 bases from a 5′- or 3′-terminus of a positive-sense single-stranded RNA virus genome, and one or more 2- to 80-nucleotide sequences from anywhere within the first 80 bases from a 5′-terminus of a negative-sense single-stranded RNA directly, where two or more (e.g., three, four, five, six, or all) of the individual 2- to 80-nucleotide sequences directly abut each other (e.g., the 5′ end of a first sequence directly abuts the 3′ end of a second sequence). In some embodiments, the nucleic acid of 10 to 200 nucleotides contains one or more 2- to 80-nucleotide sequences from anywhere within the first 80 bases from a 5′- or 3′-terminus of a positive-sense single-stranded RNA virus genome, and one or more 2- to 80-nucleotide sequences from anywhere within the first 80 bases from a 5′-terminus of a negative-sense single-stranded RNA directly, where two or more (e.g., three, four, five, six, or all) of the individual 2- to 80-nucleotide sequences do not directly abut each other (e.g., one or more (e.g., 2-5, 6-10, 11-15, or 16-20) nucleotides are present between the 5′ end of a first sequence and the 3′ end of a second 2- to 80-nucleotide sequence).

The nucleic acid compositions described herein can be single-stranded or double-stranded, including partially double-stranded. When the oligonucleotide includes double-stranded nucleic acid, the double-stranded portion includes sufficient complementary sequence to maintain the double-stranded structure under physiological conditions. This can include a plurality of adjacent or nonadjacent base pairs chosen from G-C, A-U, A-T, G-T, and G-U. In one embodiment the base pairs are chosen from G-C, A-U, and G-U. The double-stranded structure can involve RNA-RNA duplex formation, RNA-DNA duplex formation, DNA-DNA duplex formation, or duplex formation involving at least one chimeric RNA:DNA sequence (i.e., chimeric RNA:DNA-DNA duplex, chimeric RNA:DNA-RNA duplex, or chimeric RNA:DNA-chimeric RNA:DNA duplex).

For use in the compositions and methods described herein, oligonucleotides can be synthesized de novo using any of a number of procedures well known in the art, for example, the beta-cyanoethyl phosphoramidite method (Beaucage et al., 1981, Tetrahedron Lett. 22:1859); or the nucleoside H-phosphonate method (Garegg et al., 1986, Tetrahedron Lett. 27:4051-4; Froehler et al., 1986, Nucleic Acids Res. 14:5399-407; Garegg et al., 1986, Tetrahedron Lett. 27:4055-8; Gaffney et al., 1988, Tetrahedron Lett. 29:2619-22). These chemistries can be performed by a variety of automated nucleic acid synthesizers available in the market. These oligonucleotides are referred to as synthetic oligonucleotides. An isolated oligonucleotide generally refers to an oligonucleotide which is separated from components with which it is normally associated in nature. As an example, an isolated oligonucleotide can be one which is separated from a cell, from a nucleus, from mitochondria or from chromatin. In one embodiment an isolated oligonucleotide is a synthetic oligonucleotide.

Modified backbones such as phosphorothioates can be synthesized using automated techniques employing either phosphoramidate or H-phosphonate chemistries. Aryl- and alkyl-phosphonates can be made, e.g., as described in U.S. Pat. No. 4,469,863 (herein incorporated by reference); and alkylphosphotriesters (in which the charged oxygen moiety is alkylated as described in U.S. Pat. No. 5,023,243 and European Patent No. 092,574 (herein incorporated by reference)) can be prepared by automated solid phase synthesis using commercially available reagents. Methods for making other DNA and RNA backbone modifications and substitutions have been described (e.g., Uhlmann et al., 1990, Chem. Rev. 90:544; Goodchild, 1990, Bioconjugate Chem. 1:165).

In some embodiments, the immunostimulatory nucleic acid molecules described herein can be conjugated with another agent. In some embodiments, an agent that can be conjugated with a nucleic acid molecule described herein can be a TLR ligand, including, without limitation, another nucleic acid molecule described herein. In some embodiments an agent that can be conjugated with the nucleic acid molecule described herein can be an immunostimulatory nucleic acid molecule that is not an immunostimulatory nucleic acid described herein. For example, the other agent can be a CpG-DNA molecule (see, for example, U.S. Pat. Nos. 6,194,388; 6,207,646; 6,214,806; 6,218,371; 6,239,116; 6,339,086; 6,406,705; 6,429,199; and 6,653,292; each of which is herein incorporated by reference). In some embodiments, an agent that can be conjugated with a nucleic acid molecule described herein can be a TLR agonist. A TLR agonist is any agent that induces or augments a TLR-mediated signal TLR agonists include, e.g., a small molecule such as R-837 (imiquimod) or R-848 (resiquimod). In some embodiments, an agent that can be conjugated with a nucleic acid molecule described herein can be a TLR antagonist. A TLR antagonist is any agent that inhibits a TLR-mediated signal. In some embodiments, a TLR antagonist is a small molecule (see, for example, U.S. Pat. Nos. 6,221,882; 6,399,630; and 6,479,504; each of which is herein incorporated by reference) as well as certain immuno-inhibitory oligonucleotides (see, for example, Lenert et al., 2001, Antisense Nucleic Acid Drug Dev. 11:247-56; Stunz et al., 2002, Eur. J. Immunol. 32:1212-22; Lenert et al., 2003, Antisense Nucleic Acid Drug Dev. 13:143-50; and Lenert et al., 2003, DNA Cell Biol. 22:621-31).

In some embodiments, an agent that can be conjugated with a nucleic acid molecule described herein is an antigen, including an antigen per se or a nucleic acid molecule that encodes an antigen. In some embodiments, an agent that can be conjugated with a nucleic acid molecule described herein can be a medicament. In each of these embodiments, an immunostimulatory nucleic acid molecule described herein can be conjugated with the other agent through any suitable direct or indirect physicochemical linkage. In some embodiments, the linkage is a covalent bond. In some embodiments an immunostimulatory nucleic acid molecule described herein can be conjugated with the other agent through a linker.

A composition can include a conjugate of an antigen or other therapeutic agent and an isolated immunostimulatory oligonucleotide described herein. In some embodiments, the antigen or other therapeutic agent is linked directly to the immunostimulatory oligonucleotide, for example through a covalent bond. In some embodiments, the antigen or other therapeutic agent is linked indirectly to the immunostimulatory oligonucleotide, for example through a linker. When the antigen or other therapeutic agent of the conjugate is a polynucleotide encoding a peptide or polypeptide, the antigen or other therapeutic agent and the isolated immunostimulatory oligonucleotide can be incorporated into a single expression vector. When the antigen or other therapeutic agent of the conjugate is a preformed polypeptide or polysaccharide, the antigen or other therapeutic agent and the isolated immunostimulatory oligonucleotide can be linked using methods well known in the art.

In some embodiments, an immunostimulatory nucleic acid molecule described herein is conjugated with the antigen or other therapeutic agent through a linkage that involves the 3′ end of the nucleic acid molecules described herein. In some embodiments, the immunostimulatory nucleic acid molecules is conjugated with the antigen or other therapeutic agent through a linkage that involves the 5′ end of the nucleic acid molecule.

In some embodiments, an immunostimulatory nucleic acid molecule described herein is conjugated with the antigen or other therapeutic agent through a linkage that does not involve the 3′ end of the nucleic acid molecule. In other embodiments, the immunostimulatory nucleic acid molecule is conjugated with the antigen or other therapeutic agent through a linkage that does not involve the 5′ end of the nucleic acid molecule.

For administration in vivo, immunostimulatory nucleic acid molecules described herein can be associated with a molecule that results in higher affinity binding to target cell (e.g., B cell, monocytic cell, NK cell, dendritic cell) surfaces and/or increased cellular uptake by target cells to form a “nucleic acid delivery complex.” Nucleic acids can be ionically or covalently associated with appropriate molecules using techniques which are well known in the art. A variety of coupling or crosslinking agents can be used, e.g., protein A, carbodiimide, and N-succinimidyl-3-(2-pyridyldithio) propionate (SPDP). Nucleic acids can alternatively be encapsulated in liposomes or virosomes using well-known techniques.

In some embodiments, the immunostimulatory nucleic acid molecules described herein are mixed with or otherwise associated with a condensing agent, e.g., a cationic lipid. Immunostimulatory nucleic acid molecules described herein that are mixed with or otherwise associated with a cationic lipid can take the form of cationic lipid/nucleic acid complexes, including liposomes. Although immunostimulatory nucleic acid molecules described herein are biologically active when used alone (i.e., as “naked” oligonucleotides), association with one or more different cationic lipids can increase biological activity of the immunostimulatory nucleic acid molecules described herein. Without meaning to be bound to any particular theory or mechanism, it is believed that the increased biological activity associated with the use of cationic lipids is due to increased efficiency of cellular uptake of the immunostimulatory nucleic acid molecules described herein. Such lipids are commonly used for transfection applications in molecular biology. Cationic lipids can include, without limitation, DOTAP (N-[1-(2,3-dioleoyloxy)propyl]-N,N,N-trimethyla-mmonium methylsulfate), DOTMA (N-[1-(2,3-dioleoyloxy)propyl]-N,N,N-trimeth-ylammonium chloride), DOSPA (2,3-dioleyloxy-N-[2(sperminecarboxamido)ethyl-]-N,N-dimethyl-1-propanaminium trifluoroacetate), DMRIE (N,N-dimethyl-2,3-bis(tetradecyloxy)-1-propanaminium bromide), DOGS (dioctadecylamidoglycyl spermine), cholesterol, and liposomes, and any combination thereof.

Other exemplary condensing agents include other types of cationic moieties, including, for example, polycationic peptides (e.g., polyarginine, polylysine, polyarginine/polylysine, and protamine), spermine, spermidine, polyamine, pseudopeptide-polyamine, peptidomimetic polyamine, dendrimer polyamine, arginine, amidine, cationic lipid, cationic porphyrin, quarternary salt of a polyamine, or an alpha helical peptide.

In each of the foregoing aspects, the immunostimulatory nucleic acid molecules described herein can be present optionally as a salt or hydrate of the free nucleic acid.

In each of the foregoing aspects, the composition can also further include a pharmaceutically acceptable carrier, such that this disclosure also provides pharmaceutical compositions containing the isolated immunostimulatory oligonucleotides described herein. Such pharmaceutical compositions can be prepared by placing an isolated immunostimulatory oligonucleotide described herein in contact with a pharmaceutically acceptable carrier.

### Antigens

The disclosed compositions and methods are applicable to a wide variety of antigens. In some embodiments, the antigen is a protein, polypeptide, or peptide. In other embodiments the antigen is DNA. The antigen can also be a lipid, a carbohydrate, or an organic molecule, in particular a small organic molecule. Exemplary antigens are disclosed in US 2003/0099668 and WO 03/024481, both of which are incorporated herein by reference. The compositions provided herein can contain two or more antigens (e.g., at least three, four, five, or six antigens).

Antigens can be selected from the group consisting of the following: (a) polypeptides suited to induce an immune response against cancer cells; (b) polypeptides suited to induce an immune response against infectious diseases; (c) polypeptides suited to induce an immune response against allergens; (d) polypeptides suited to induce an immune response in farm animals or pets; and (e) fragments (e.g., a domain) of any of the polypeptides set out in (a)-(d).

Exemplary antigens include those from a pathogen (e.g. virus, bacterium, parasite, fungus) and tumors (especially tumor-associated antigens or “tumor markers”). Other exemplary antigens include autoantigens.

In some embodiments, the antigen or antigenic determinant is one that is useful for the prevention of infectious disease. Such treatment will be useful to treat a wide variety of infectious diseases affecting a wide range of hosts, e.g., human, cow, sheep, pig, dog, cat, and other mammalian species and non-mammalian species as well. Thus, antigens or antigenic determinants selected for the compositions will be well known to those in the medical art.

Examples of antigens or antigenic determinants include the following: the HIV antigens gp140 and gp160; the influenza antigens hemagglutinin, M2 protein, and neuraminidase; hepatitis B surface antigen or core; and circumsporozoite protein of malaria, or fragments thereof.

As discussed above, antigens include infectious microbes such as viruses, bacteria, and fungi, and fragments thereof, obtained or derived from natural sources or synthetically. Infectious viruses of both human and non-human vertebrates include retroviruses, RNA viruses, and DNA viruses. The group of retroviruses includes both simple retroviruses and complex retroviruses. The simple retroviruses include the subgroups of B-type retroviruses, C-type retroviruses, and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, T-cell leukemia viruses, and the foamy viruses. Lentiviruses include HIV-1, but also include HIV-2, SIV, Visna virus, feline immunodeficiency virus (FIV), and equine infectious anemia virus (EIAV). The T-cell leukemia viruses include HTLV-1, HTLV-T1, simian T-cell leukemia virus (STLV), and bovine leukemia virus (BLV). The foamy viruses include human foamy virus (HFV), simian foamy virus (SFV), and bovine foamy virus (BFV).

Polypeptides of bacterial pathogens include, but are not limited to, an iron-regulated outer membrane protein (TROMP), an outer membrane protein (OMP), and an A-protein of Aeromonis salmonicida which causes furunculosis, p57 protein of Renibacterium salmoninarum which causes bacterial kidney disease (BKD), major surface associated antigen (msa), a surface expressed cytotoxin (mpr), a surface expressed hemolysin (ish), and a flagellar antigen of Yersinia; an extracellular protein (ECP), an iron-regulated outer membrane protein (TROMP), and a structural protein of Pasteurella; an OMP and a flagellar protein of Vibrio anguillarum and V. ordalii; a flagellar protein, an OMP protein, aroA, and purA of Edwardsiella ictaluri and E. tarda; and surface antigen of Ichthyophthirius; and a structural and regulatory protein of Cytophaga columnari; and a structural and regulatory protein of Rickettsia.

The selection of antigens or antigenic determinants for compositions and methods of treatment for drug addiction, in particular recreational drug addiction, would be known to those skilled in the medical arts treating such disorders. Representative examples of such antigens or antigenic determinants include, for example, opioids and morphine derivatives, such as codeine, fentanyl, heroin, morphium, and opium; stimulants such as amphetamine, cocaine, MDMA (methylenedioxymethamphetamine), methamphetamine, methylphenidate, and nicotine; hallucinogens, such as LSD, mescaline, and psilocybin; as well as cannabinoids, such as hashish and marijuana.

The selection of antigens or antigenic determinants for compositions and methods of treatment for other diseases or conditions associated with self antigens would be also known to those skilled in the medical arts treating such disorders. Representative examples of such antigens or antigenic determinants are, for example, lymphotoxins (e.g. lymphotoxin alpha (LT-alpha), and lymphotoxin beta (LT-beta)), and lymphotoxin receptors, receptor activator of nuclear factor kappaB ligand (RANKL), vascular endothelial growth factor (VEGF), and VEGF receptor (VEGF-R), interleukin 17, and amyloid beta peptide (Aβ1-42), TNF-α, macrophage migration inhibitory factor (MTF), monocyte chemo-attractant protein-1 (MCP-1), stromal cell-derived factor-1 (SDF-1), Rank-L, macrophage-colony stimulating factor (M-CSF), Angiotensin II, Endoglin, Eotaxin, Grehlin, B-lymphocyte chemoattractant (BLC), chemokine ligand 21 (CCL21) interleukin-13(IL-13), interleukin-17 (IL-17), interleukin-5 (IL-5), interleukin-8 (IL-8), interleukin-15 (IL-15), Bradykinin, Resistin, luteinizing hormone-releasing hormone (LHRH), growth hormone-releasing hormone (GHRH), growth inhibiting hormone (GIH), corticotropin-releasing hormone (CRH), thyrotropin-releasing hormone (TRH), and Gastrin, as well as fragments of each which can be used to elicit immunological responses.

In some embodiments, the antigen or antigenic determinant is selected from the group consisting of: (a) a recombinant polypeptide of HIV; (b) a recombinant polypeptide of Influenza virus (e.g., an Influenza virus M2 polypeptide or a fragment thereof); (c) a recombinant polypeptide of Hepatitis C virus; (d) a recombinant polypeptide of Hepatitis B virus (e) a recombinant polypeptide of Toxoplasma; (f) a recombinant polypeptide of Plasmodium falciparum; (g) a recombinant polypeptide of Plasmodium vivax; (h) a recombinant polypeptide of Plasmodium ovale; (i) a recombinant polypeptide of Plasmodium malariae; (j) a recombinant polypeptide of agent/allergen of bee sting allergy; (k) a recombinant polypeptide of agent/allergen of nut allergy; (l) a recombinant polypeptide of agent/allergen of pollen allergy; (m) a recombinant polypeptide of house dust mite; (n) a recombinant polypeptide of agents of (allergens responsible for) cat or cat hair allergy; (o) a recombinant protein of agents of food allergies; (p) a recombinant protein of asthma allergens; (q) a recombinant protein of Chlamydia; and (r) a fragment of any of the proteins set out in (a)-(q).

In some embodiments, the antigen is a tumor-associated antigen. Non-limiting examples of tumor-associated antigens include melanoma antigen E (MAGE)-3, MAGE-C1, MAGE-B1, MAGE-B2, MAGE-2, MAGE-4A, MAGE-4B, mucin (MUC)-1, MUC-2, human telomerase reverse transcriptase (hTERT), cytokeratin fragment 19 (CYFRA 21-1), squamous cell carcinoma antigen 1 (SCCA-1), squamous cell carcinoma antigen 2 (SCCA-2), ovarian carcinoma antigen CA125 (1A1-3B), Mucin 1, cutaneous T-cell lymphoma (CTCL) antigen se1-1, CTCL antigen se14-3, CTCL antigen se20-4, CTCL antigen se20-9, CTCL antigen se33-1, CTCL antigen se37-2, CTCL antigen se57-1, CTCL antigen se89-1, prostate-specific membrane antigen, 5T4 oncofetal trophoblast glycoprotein, Orf73 Kaposi's sarcoma-associated herpesvirus, colon cancer antigen NY-CO45, lung cancer antigen NY-LU-12 variant A, cancer associated surface antigen, adenocarcinoma antigen ART1, paraneoplastic associated brain-testis-cancer antigen, neuro-oncological ventral antigen-2 (NOVA2), hepatocellular carcinoma antigen gene 520, tumor-associated antigen MAGE-X2, synovial sarcoma, X breakpoint 2, breast cancer antigen NY-BR-15, breast cancer antigen BY-BR-16, chromograninin A, alpha-fetoprotein (AFP), carcinoembryonic antigen (CEA), epithelial tumor antigen (ETA), tyrosinase, 707 alanine proline (707-AP), interferon-inducible protein absent in melanoma 2 (AIM-2), adenocarcinoma antigen recognized by T cells 4 (ART-4), B-antigen (BAGE), breakpoint cluster region Abelson (Bcr-abl), CTL-recognized antigen on melanoma (CAMEL), carcinoembryonic antigen peptide-1 (CAP-1), caspase-8 (CASP-8), cell-division cycle 27 (CDC27), cyclin-dependent kinase 4 (CDK4), carcino-embryonic antigen (CEA), calcium-activated chloride channel-2 (CLCA2), cancer/testis (CT) antigen, cyclophilin B (Cyp-B), elongation factor 2 (ELF2), epithelial cell adhesion molecule (Ep-CAM), Ephrin type-A receptor 2, 3 (EphA2, 3), Ets variant gene 6/acute myeloid leukemia 1 gene ETS (ETV6-AML1), fibroblast growth factor-5 (FGF-5), fibronectin (FN), glycoprotein 250, G antigen (GAGE), N-acetylglucosaminyltransferase V (GnT-V), glycoprotein 100 kD (Gp100), helicase antigen (HAGE), human epidermal receptor-2/neurological (HER-2/neu), heat shock protein 70-2 mutated (HSP70-2M), human signet ring tumor-2 (HST-2), intestinal carboxyl esterase (iCE), interleukin 13 receptor α2 chain (IL-13Rα2), L antigen (LAGE), low density lipid receptor/GDP-L-fucose (LDLR/FUT), melanoma antigen recognized by T cells-1/Melanoma antigen A (MART-1/Melan-A), melanoma Ag recognized by T cells-2 (MART-2), melanocortin 1 receptor (MC1R), macrophage colony-stimulating factor gene (M-CSF), melanoma ubiquitous mutated (MUM)-1, MUM-2, MUM-3, Neo-poly(A) polymerase (Neo-PAP), nucleophosmin/anaplastic lymphoma kinase fusion protein (NPM/ALK), New York-esophagus 1 (NY-ESO-1), ocular albinism type 1 protein (OA1), O-linked N-acetylglucosamine transferase gene (OS-9), protein 15 (P15), p190 minor bcr-abl, promyelocytic leukemia/retinoic acid receptor α (Pml/RARα), preferentially expressed antigen of melanoma (PRAME), prostate specific antigen (PSA), prostate-specific membrane antigen (PSMA), receptor-type protein-tyrosine phosphatase kappa (PTPRK), renal antigen (RAGE), renal ubiquitous (RU)-1, RU-2, sarcoma antigen (SAGE), squamous antigen rejecting tumor (SART)-1, SART-2, SART-3, intron 2-retaining surviving (Survivin-2B), synaptotagmin Usynovial sarcoma, X fusion protein (SYT/SSX), translocation Ets-family leukemia/acute myeloid leukemia 1 (TEL/AML1), transforming growth factor β receptor 2 (TGFβRII), triosephosphate isomerase (TPI), taxol resistant associated protein 3 (TRAG-3), testin-related gene (TRG), tyrosinase related protein (TRP)-1, TRP-2, TRP-2/intron 2 (TRP-2/INT2), TRP-2/novel exon 6b (TRP-2/6b), and Wilms' tumor gene (WT1). In some embodiments, the compositions provided herein contain two or more tumor-associated antigens that are present or associated with a particular type of cancer (e.g., breast cancer, bladder cancer, colon cancer, rectal cancer, endometrial cancer, kidney cancer, lung cancer, melanoma, non-Hodgkin lymphoma, pancreatic cancer, prostate cancer, or thyroid cancer).

### Compositions

The compositions described herein can include a variety of materials and substances in addition to the recited nucleic acids. As noted elsewhere herein, such materials and substances can include adjuvants, antigens, carriers, pharmaceutically acceptable excipients, and the like.

In some embodiments, carriers useful in the compositions and methods disclosed herein include protein carriers or synthetic nanocarriers. In some embodiments, synthetic nanocarriers comprising the compositions are coupled to an antigen. In additional embodiments, such synthetic nanocarriers further include an additional synthetic nanocarrier not coupled to the immunostimulatory isolated nucleic acid, and in some embodiments the additional synthetic nanocarrier is coupled to an antigen.

In some embodiments, the compositions are administered together with conjugate, or non-conjugate, vaccines. In some embodiments, the compositions include a carrier peptide or protein, or to another type of carrier. Useful carriers include carrier proteins known to be useful in conjugate vaccines, including but not limited to tetanus toxoid (TT), diphtheria toxoid (DT), the nontoxic mutant of diphtheria toxin, CRM197, the outer membrane protein complex from group B N. meningitidis, and keyhole limpet hemocyanin (KLH). Other carriers can include the synthetic nanocarriers described elsewhere herein, and other carriers that might be known conventionally.

Coupling of antigens or the recited nucleic acids to carriers can be performed using conventional covalent or non-covalent coupling techniques. Useful techniques for developing conjugated vaccines include, but are not limited to, those generally described in Lairmore et al., 1995, J. Virol., 69(10):6077-89; Rittershause, 2000, Arterioscler. Thromb. Vasc. Biol., 20(9):2106-12; Chengalvala et al., 1999, Vaccine, 17(9-10):1035-41; Dakappagari et al., 2003, J. Immunol., 170(8):4242-53; Garrett et al., 2007, J. Immunol., 178(11):7120-31.

In other embodiments, the compositions described herein can be combined with antigen, or a conventional vaccine, in a vehicle to form an injectable mixture. The mixtures can be made using conventional pharmaceutical manufacturing and compounding techniques to arrive at useful dosage forms. Techniques suitable for use in practicing the present compositions and methods can be found in a variety of sources, including, but not limited to, Powell et al., Vaccine Design, 1995, Springer-Verlag; or Paoletti et al., Eds., Vaccines: from Concept to Clinic: A Guide to the Development and Clinical Testing of Vaccines for Human Use, 1999, CRC Press.

In some embodiments, synthetic nanocarriers are used as carriers. A wide variety of synthetic nanocarriers can be used. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size, shape, and/or composition so that each synthetic nanocarrier has similar properties. For example, at least 80%, at least 90%, or at least 95% of the synthetic nanocarriers, based on the total number of synthetic nanocarriers, can have a minimum dimension or maximum dimension that falls within 5%, 10%, or 20% of the average diameter or average dimension of the synthetic nanocarriers. In some embodiments, a population of synthetic nanocarriers can be heterogeneous with respect to size, shape, and/or composition.

In various embodiments, a minimum dimension of at least 75%, e.g., at least 80% or at least 90%, of the synthetic nanocarriers in a sample, based on the total number of synthetic nanocarriers in the sample, is greater than 100 nm. In an embodiment, a maximum dimension of at least 75%, e.g., at least 80%, or at least 90%, of the synthetic nanocarriers in a sample, based on the total number of synthetic nanocarriers in the sample, is equal to or less than 5 μm. In some embodiments, a minimum dimension of at least 75%, at least 80%, or at least 90%, of the synthetic nanocarriers in a sample, based on the total number of synthetic nanocarriers in the sample, is greater than 110 nm, e.g., greater than 120 nm, greater than 130 nm, or greater than 150 nm. Aspect ratios of the maximum and minimum dimensions of synthetic nanocarriers can vary depending on the embodiment. For instance, aspect ratios of the maximum to minimum dimensions of the synthetic nanocarriers can vary from 1:1 to 1,000,000:1, e.g., from 1:1 to 100,000:1, from 1:1 to 1000:1, from 1:1 to 100:1, or from 1:1 to 10:1. In some embodiments, a maximum dimension of at least 75%, at least 80%, or at least 90% of the synthetic nanocarriers in a sample, based on the total number of synthetic nanocarriers in the sample is equal to or less than 3 μm, equal to or less than 2 μm, equal to or less than 1 μm, equal to or less than 800 nm, equal to or less than 600 nm, or equal to or less than 500 nm. In some embodiments, a maximum dimension of at least 75%, at least 80%, or at least 90% of the synthetic nanocarriers in a sample, based on the total number of synthetic nanocarriers in the sample, is equal to or greater than 100 nm, equal to or greater than 120, greater than 130 nm, greater than 140 nm, or greater than 150 nm. Measurement of synthetic nanocarrier sizes is obtained by suspending the synthetic nanocarriers in a liquid (usually aqueous) media and using dynamic light scattering (e.g., using a Brookhaven ZetaPALS instrument).

Synthetic nanocarriers can be solid or hollow, and can include one or more layers. In some embodiments, each layer has a unique composition and unique properties relative to the other layer(s). To give but one example, synthetic nanocarriers can have a core/shell structure, wherein the core is one layer (e.g., a polymeric core) and the shell is a second layer (e.g., a lipid bilayer or monolayer). Synthetic nanocarriers can include a plurality of different layers.

In some embodiments, synthetic nanocarriers can optionally include one or more lipids. In some embodiments, a synthetic nanocarrier can include a liposome. In some embodiments, a synthetic nanocarrier can include a lipid bilayer. In some embodiments, a synthetic nanocarrier can include a lipid monolayer. In some embodiments, a synthetic nanocarrier can include a micelle. In some embodiments, a synthetic nanocarrier can include a core comprising a polymeric matrix surrounded by a lipid layer (e.g., lipid bilayer, lipid monolayer, etc.). In some embodiments, a synthetic nanocarrier can include a non-polymeric core (e.g., metal particle, quantum dot, ceramic particle, bone particle, viral particle, proteins, nucleic acids, carbohydrates, etc.) surrounded by a lipid layer (e.g., lipid bilayer, lipid monolayer, etc.).

In some embodiments, synthetic nanocarriers can include one or more polymers. In some embodiments, such a polymer can be surrounded by a coating layer (e.g., liposome, lipid monolayer, micelle, etc.). In some embodiments, various elements of the synthetic nanocarriers can be coupled with the polymer.

In some embodiments, an antigen, targeting moiety, and/or nucleic acid are covalently associated with a polymeric matrix. In some embodiments, covalent association is mediated by a linker. In some embodiments, an antigen, targeting moiety, and/or oligonucleotide can be noncovalently associated with a polymeric matrix. For example, in some embodiments, an antigen, targeting moiety, and/or nucleic acid can be encapsulated within, surrounded by, and/or dispersed throughout a polymeric matrix. Alternatively or additionally, an antigen, targeting moiety, and/or nucleotide can be associated with a polymeric matrix by hydrophobic interactions, charge interactions, van der Waals forces, etc.

A wide variety of polymers and methods for forming polymeric matrices therefrom are known conventionally. In general, a polymeric matrix includes one or more polymers. Polymers can be natural or unnatural (synthetic) polymers. Polymers can be homopolymers or copolymers comprising two or more monomers. In terms of sequence, copolymers can be random, block, or include a combination of random and block sequences. Typically, polymers in accordance with the present disclosure are organic polymers.

Examples of polymers suitable for use in the present compositions and methods include, but are not limited to, polyethylenes, polycarbonates (e.g. poly(1,3-dioxan-2one)), polyanhydrides (e.g. poly(sebacic anhydride)), polypropylfumarates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), and poly(ethylene imine)-PEG copolymers.

In some embodiments, polymers in accordance with the present disclosure include polymers that have been approved for use in humans by the U.S. Food and Drug Administration (FDA) under 21 C.F.R. §177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers are hydrophilic. For example, polymers can include anionic groups (e.g., phosphate group, sulfate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers are hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer can have an impact on the nature of materials that are incorporated (e.g. coupled) within the synthetic nanocarrier.

In some embodiments, polymers can be modified with one or more moieties and/or functional groups. A variety of moieties or functional groups can be used in accordance with the present compositions and methods. In some embodiments, polymers can be modified with polyethylene glycol (PEG), with a carbohydrate, and/or with acyclic polyacetals derived from polysaccharides (Papisov, 2001, ACS Symposium Series, 786:301). Certain embodiments can be made using the general teachings of U.S. Pat. No. 5,543,158, or WO 2009/051837 (each of which is incorporated by reference).

In some embodiments, polymers can be modified with a lipid or fatty acid group. In some embodiments, a fatty acid group can be one or more of butyric, caproic, caprylic, capric, lauric, myristic, palmitic, stearic, arachidic, behenic, or lignoceric acid. In some embodiments, a fatty acid group can be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers can be polyesters, including copolymers comprising lactic acid and glycolic acid units, such as poly(lactic acid-co-glycolic acid) and poly(lactide-co-glycolide), collectively referred to herein as “PLGA;” and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof). In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer can be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present compositions and methods is characterized by a lactic acid:glycolic acid ratio of approximately 85:15, approximately 75:25, approximately 60:40, approximately 50:50, approximately 40:60, approximately 25:75, or approximately 15:85.

In some embodiments, the polymers are or include one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer can include fully-polymerized copolymers of acrylic and methacrylic acid esters with a low content of quaternary ammonium groups.

In some embodiments, polymers are or include cationic polymers. In general, cationic polymers are able to condense and/or protect negatively charged strands of nucleic acids (e.g., DNA, or derivatives thereof) Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines. In some embodiments, the synthetic nanocarriers do not include (or can exclude) cationic polymers.

In some embodiments, polymers are or include degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and general methods for preparing such polymers are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929 (each of which is incorporated by reference); Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732 (each of which is incorporated by reference).

In some embodiments, polymers are or include linear or branched polymers. In some embodiments, polymers are or include dendrimers. In some embodiments, polymers are substantially cross-linked to one another. In some embodiments, polymers are substantially free of cross-links. In some embodiments, polymers are used in accordance with the present disclosure without undergoing a cross-linking step. It is further to be understood that synthetic nanocarriers can include block copolymers, graft copolymers, blends, mixtures, and/or adducts of any of the foregoing and other polymers. Those skilled in the art will recognize that the polymers listed herein represent an exemplary, not comprehensive, list of polymers that can be of use in the disclosed compositions and methods.

In some embodiments, synthetic nanocarriers do not include a polymeric component. In some embodiments, synthetic nanocarriers can include metal particles, quantum dots, ceramic particles, etc. In some embodiments, a non-polymeric synthetic nanocarrier is an aggregate of non-polymeric components, such as an aggregate of metal atoms (e.g., gold atoms).

In some embodiments, synthetic nanocarriers can optionally include one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present disclosure. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester, such as sorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present disclosure.

In some embodiments, synthetic nanocarriers can optionally include one or more carbohydrates. Carbohydrates may be natural or synthetic. A carbohydrate may be a derivatized natural carbohydrate. In certain embodiments, a carbohydrate includes monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In some embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In some embodiments, the synthetic nanocarriers do not include (or specifically exclude) carbohydrates, such as a polysaccharide. In some embodiments, the carbohydrate can include a carbohydrate derivative such as a sugar alcohol, including but not limited to mannitol, sorbitol, xylitol, erythritol, maltitol, and lactitol.

In some embodiments, when preparing carriers for use with the compositions, methods for covalently coupling the recited nucleic acids or other elements of the inventive compositions to the carriers may be useful. In some embodiments, the recited nucleic acids or other elements of the compositions can be coupled non-covalently to the carriers. If the element to be coupled includes a small molecule it can be of advantage to attach the element to a polymeric carrier prior to the assembly of synthetic nanocarriers. In some embodiments, it can be an advantage (for manufacturing or other reasons) to prepare carriers, especially synthetic nanocarriers, with surface groups that are used to couple the adjuvant to the carrier through the use of these surface groups

In some embodiments, non-covalent coupling can be accomplished using adsorption. Adsorption of nucleic acids to the surface of a nanoparticle can be accomplished by salt formation. When using this method, the nanoparticle is prepared in such a manner that the nanoparticle includes a material that introduces a charge to the nanoparticle. Often the use of a charged surfactant, a cationic surfactant is used to adsorb the negatively charged nucleic acids during the nanoparticle preparation is sufficient to provide surface charge to the nanoparticle. Contacting the charged nanoparticles with a solution of nucleic acids causes adsorption of the nucleic acids. This method is described in WO 00/06123 (herein incorporated by reference). Encapsulation of nucleic acids can be accomplished by dissolving the nucleic acids in an aqueous buffer and then using this solution in the single or double emulsion process to form nanoparticles by self-assembly. This process is described in Tse et al., 2009, Int. J. Pharmaceutics, 370 (1-2):33. Additional encapsulation methods are described elsewhere herein.

Covalent coupling can be accomplished by a number of methods. These methods are covered in detail in Bioconjugate Techniques, 2nd edition, Elsevier (2008) by Hermanson. One method that is particularly suited to coupling nucleic acids to polymers or nanoparticles carrying amine functional groups is to activate the 5′ phosphate of the nucleic acid with 1-(3-dimethylamino)propyl-3-ethylcarbodiimide methiodide (EDC) and imidazole and then allowing the activated nucleic acid to react with the amine substituted polymer or nanoparticle [Shabarova et al., 1983, FEBS Lett., 154:288].

In certain embodiments, covalent coupling can be made via a covalent linker. In some embodiments, the covalent linker can include an amide linker, a disulfide linker, a thioether linker, a hydrazone linker, a hydrazide linker, an imine or oxime linker, a urea or thiourea linker, an amidine linker, an amine linker, and a sulfonamide linker.

An amide linker is formed via an amide bond between an amine on one element with the carboxylic acid group of a second element such as the nanocarrier. The amide bond in the linker can be made using any of the conventional amide bond forming reactions with suitably protected amino acids or antigens or adjuvants and activated carboxylic acid such N-hydroxysuccinimide-activated ester.

A disulfide linker is made via the formation of a disulfide (S—S) bond between two sulfur atoms of the form, for instance, of R1-S—S—R2. A disulfide bond can be foamed by thiol exchange of an antigen or adjuvant containing thiol/mercaptan group (—SH) with another activated thiol group on an element containing thiol/mercaptan groups with an element containing an activated thiol group.

A triazole linker, specifically a 1,2,3-triazole of the form

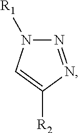

wherein R1 and R2 can be any chemical entities, is made by the 1,3-dipolar cycloaddition reaction of an azide attached to a first element with a terminal alkyne attached to a second element. The 1,3-dipolar cycloaddition reaction is performed with or without a catalyst, preferably with Cu(I)-catalyst, which links the two elements through a 1,2,3-triazole function. This chemistry is described in detail by Sharpless et al., 2002, Angew. Chem. Int. Ed. 41(14), 2596, and Meldal et al., 2008, Chem. Rev., 108(8), 2952-3015 and is often referred to as a “click reaction” or CuAAC.

A thioether linker is made by the formation of a sulfur-carbon (thioether) bond in the form, for instance, of R1-S—R2. Thioether can be made by either alkylation of a thiol/mercaptan (—SH) group on one component such as the element with an alkylating group such as halide or epoxide on a second element. Thioether linkers can also be formed by Michael addition of a thiol/mercaptan group on one element to an electron-deficient alkene group on a second element such as a polymer containing a maleimide group as the Michael acceptor. In another way, thioether linkers can be prepared by the radical thiol-ene reaction of a thiol/mercaptan group on one element with an alkene group on a second element such as a polymer or nanocarrier. A hydrazone linker is made by the reaction of a hydrazide group on one element with an aldehyde/ketone group on the second element.

A hydrazide linker is formed by the reaction of a hydrazine group on one element with a carboxylic acid group on the second element. Such reaction is generally performed using chemistry similar to the formation of amide bond where the carboxylic acid is activated with an activating reagent.

An imine or oxime linker is formed by the reaction of an amine or N-alkoxyamine (or aminooxy) group on one element with an aldehyde or ketone group on a second element.

A urea or thiourea linker is prepared by the reaction of an amine group on one element with an isocyanate or thioisocyanate group on a second element.

An amidine linker is prepared by the reaction of an amine group on one element with an imidoester group on a second element.

An amine linker is made by the alkylation reaction of an amine group on one element with an alkylating group such as halide, epoxide, or sulfonate ester group on the second element. Alternatively, an amine linker can also be made by reductive amination of an amine group on one element with an aldehyde or ketone group on the second element with a suitable reducing reagent such as sodium cyanoborohydride or sodium triacetoxyborohydride.

A sulfonamide linker is made by the reaction of an amine group on one element with a sulfonyl halide (such as sulfonyl chloride) group on a second element.

Elements can also be coupled via non-covalent coupling methods. For examples, a negative charged antigen or adjuvant can be coupled to a positively charged carrier through electrostatic adsorption. An antigen or adjuvant containing a metal ligand can also be coupled to a carrier containing a metal complex via a metal-ligand complex.

In some embodiments, an element such as the recited nucleic acids, antigen, or adjuvant is attached to a polymer, for example polylactic acid-block-polyethylene glycol, prior to the assembly of a synthetic nanocarrier or a synthetic nanocarrier is formed with reactive or activatable groups on its surface. In the latter case, the antigen or adjuvant can be prepared with a group which is compatible with the attachment chemistry that is presented by the synthetic nanocarriers' surface. In some embodiments, a peptide antigen can be attached to VLPs or liposomes using a suitable linker. A linker is a compound or reagent that is capable of coupling two molecules together. In certain embodiments, the linkers are homobifunctional or heterobifunctional reagents as described in Hermanson 2008. For example, a VLP or liposome synthetic nanocarrier containing a carboxylic group on the surface can be treated with a homobifunctional linker, adipic dihydrazide (ADH), in the presence of EDC to form the corresponding synthetic nanocarrier with the ADH linker. The resulting ADH linked synthetic nanocarrier is then conjugated with a peptide antigen containing an acid group via the other end of the ADH linker on NC to produce the corresponding VLP or liposome peptide conjugate.

Synthetic nanocarriers can be prepared using a wide variety of methods known in the art. For example, synthetic nanocarriers can be formed by methods as nanoprecipitation, flow focusing fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755, and also U.S. Pat. Nos. 5,578,325 and 6,007,845).

Various materials can be encapsulated into synthetic nanocarriers as desirable using a variety of methods including, but not limited to, C. Astete et al., “Synthesis and characterization of PLGA nanoparticles,” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006). Other methods suitable for encapsulating materials, such as nucleic acids, into synthetic nanocarriers can be used, including without limitation, the methods disclosed in U.S. Pat. No. 6,632,671; Martimprey et al., 2009, Eur. J. Pharm. Biopharm. 71:490-504; or Malyala et al., 2009, Adv. Drug Delivery Rev. 61: 218-225.

In certain embodiments, synthetic nanocarriers are prepared by a nanoprecipitation process or spray drying. Conditions used in preparing synthetic nanocarriers can be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used can depend on the materials to be coupled to the synthetic nanocarriers and/or the composition of the polymer matrix.

If particles prepared by any of the above methods have a size range outside of the desired range, particles can be sized, for example, using a sieve.

Elements of the compositions described herein (such as targeting moieties, polymeric matrices, antigens and the like) can be coupled to the overall carrier, e.g., by one or more covalent bonds, or can be coupled by means of one or more linkers. Methods of functionalizing synthetic nanocarriers can be adapted from U.S. Patent Application Publication Nos. 2006/0002852 and 2009/0028910, or WO 2008/127532 (each of which is incorporated by reference).

Alternatively or additionally, carriers can be coupled to the recited nucleic acids, targeting moieties, adjuvants, various antigens, and/or other elements directly or indirectly via non-covalent interactions. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such couplings can be arranged to be on a portion of a carrier, such as an external surface or an internal surface of a synthetic nanocarrier. In some embodiments, encapsulation and/or absorption is a form of coupling.

In some embodiments, the compositions described herein can include certain adjuvants, in addition to the immunostimulatory isolated nucleic acids, through admixing in the same vehicle or delivery system. Such adjuvants can include, but are not limited to mineral salts, such as alum, alum combined with monophosphoryl lipid (MPL) A of Enterobacteriaceae, such as Escherichia coli, Salmonella minnesota, Salmonella typhimurium, or Shigella flexneri or specifically with MPL® (AS04), MPL A of above-mentioned bacteria separately, saponins, such as QS-21, Quil-A, ISCOMs, ISCOMATRIX™, emulsions, such as MF59™, MONTANIDE® ISA 51 and ISA 720, AS02 (QS21+squalene+MPL®), liposomes and liposomal formulations such as AS01, synthesized or specifically prepared microparticles and microcarriers, such as bacteria-derived outer membrane vesicles (OMV) of Neisseria meningitidis, N. gonorrheae, Francisella novicida and others, or chitosan particles, depot-forming agents, such as PLURONIC® block co-polymers, specifically modified or prepared peptides, such as muramyl dipeptide, aminoalkyl glucosaminide 4-phosphates, such as RC529, or proteins, such as bacterial toxoids or toxin fragments. The doses of such other adjuvants can be determined using conventional dose ranging studies.

In some embodiments, the compositions described herein can be combined with an antigen. Such an antigen can be different from, or similar or identical to those coupled to a nanocarrier (with or without adjuvant, utilizing or not utilizing another delivery vehicle) administered separately at a different time-point and/or at a different body location and/or by a different immunization route or with another antigen and/or adjuvant-carrying composition administered separately at a different time-point and/or at a different body location and/or by a different immunization route.

The compositions described herein can be made using conventional pharmaceutical manufacturing and compounding techniques to arrive at useful dosage forms. Techniques suitable for use in practicing the present disclosure can be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In some embodiments, the compositions described herein are formulated in sterile saline solution for injection together with a preservative.

It is to be understood that the compositions described herein can be made in any suitable manner, and the new compositions described herein are in no way limited to compositions that can be produced using the methods described herein. Selection of an appropriate method can require attention to the properties of the particular materials and substances being utilized.

### Methods and Uses

The compositions and methods described herein can be used to induce, enhance, suppress, modulate, direct, or redirect an immune response. For example, in some embodiments the methods result in the induction or enhancement of a Th1-like immune response, a proinflammatory immune response, or an antigen-specific immune response in a subject (e.g., a human). The compositions and methods described herein can be used in the diagnosis, prophylaxis, and/or treatment of conditions such as cancers, infectious diseases, metabolic diseases, degenerative diseases, autoimmune diseases, inflammatory diseases, immunological diseases, or other disorders and/or conditions. The compositions and methods described herein can also be used for the prophylaxis and/or treatment of a condition resulting from the exposure to a toxin, hazardous substance, environmental toxin, or other harmful agent.

In some embodiments, the subject (e.g., a human) may be diagnosed with a condition (e.g., a cancer (e.g., any of the cancers described herein), a metabolic disease (e.g., Addison's disease, Hashimoto's disease, Cushing's disease, acid lipase disease, Barth syndrome, Central Pontine Myelinolysis, Farber's disease, G6PH Deficiency, Gangliosidoses, Hunter syndrome, hypophosphatasia, Lesch-Nyhan syndrome, lipid storage diseases, metabolic myopathies, mitochondrial myopathies, mucolipidoses, mucopolysaccharidoses, Pompe disease, type I glycogen storage disease, type II diabetes, and metabolic syndrome X), a degenerative disease (e.g., Alzheimer's disease, Parkinson's disease, Huntington's disease, and amyotrophic lateral sclerosis), an autoimmune disease (e.g., Sjogren syndrome, Celiac disease, dermatomyositis, Hashimoto's thyroiditis, multiple sclerosis, myasthenia gravis, pernicious anemia, reactive arthritis, rheumatoid arthritis, systemic lupus erythematosus, and type I diabetes), an inflammatory disease (e.g., irritable bowel syndrome, nephritis, ulcerative colitis, hepatitis, Crohn's disease, atherosclerosis, and asthma), or an immunological disease (e.g., allergy, immune complex diseases, and transplant rejection) or be determined to have an increased risk of developing a condition (e.g., a cancer, a metabolic disease, a degenerative disease, an autoimmune disease, an inflammatory disease, or an immunological disease), e.g., based on a genetic predisposition known to be associated with a specific disorder. In any of the methods described herein, a subject can be administered at least one dose (e.g., at least two, three, four, five, or six) doses of any of the compositions described herein.

For example, provided herein are methods of treating or reducing the risk of developing a cancer in a subject. These methods include administering to the subject at least one dose of any of the compositions described herein (e.g., any of the compositions containing at least one tumor-associated antigen) in an amount effective to treat or reduce the risk of a cancer in the subject.

Non-limiting examples of cancers that can be treated by the methods described herein include leukemia, adrenocortical carcinoma, Kaposi sarcoma, lymphoma, anal cancer, appendix cancer, astrocytomas, basal cell carcinoma, bile duct cancer, bone cancer, breast cancer, bronchial tumors, Burkitt lymphoma, carcinoid tumor, cervical cancer, chordoma, chronic lymphocytic leukemia, colon cancer, colorectal cancer, craniopharyngioma, lymphoma, endometrial cancer, ependymoblastoma, ependymoma, esophageal cancer, Ewing sarcoma, liver cancer, intraocular melanoma, retinoblastoma, kidney cancer, Kaposi sarcoma, lip and oral cavity cancer, lung cancer, mesothelioma, mouth cancer, myeloma, nasopharyngeal cancer, rectal cancer, sarcoma, skin cancer, stomach cancer, testicular cancer, throat cancer, thymic carcinoma, thyroid cancer, and uterine cancer. In some embodiments, a subject having a specific form of cancer is administered at least one dose of a composition (e.g., any of the compositions described herein) that contain at least one antigen associated with the specific form of cancer.

In some embodiments, the methods of treatment result in a decrease (e.g., a detectable or observable decrease) in the number of symptoms experienced by a subject (e.g., a human) having a cancer (e.g., as compared to a subject having the same cancer and receiving a different treatment or no treatment, or compared to the number of symptoms experienced by the subject prior to the start of the treatment) and/or a reduction in the size of a solid tumor and/or a reduction in the number of circulating tumor cells (CTCs) found in the subject's blood. In some embodiments, the methods of treatment result in a decrease in the severity, frequency, and/or duration of one or more symptoms in the subject (e.g., as compared to a subject having the same cancer and receiving a different treatment or no treatment, or compared to the symptoms experienced by the subject prior to the start of the treatment). The symptoms associated with specific forms of cancer are known by those skilled in the art.

In some embodiments, the methods of treatment result in a decrease in the risk of developing a cancer in a subject. For example, the methods of treatment can result in a decrease in the risk of developing a cancer in a subject, as compared to a control subject or a control population having the same risk factors for developing a cancer but receiving a different treatment or no treatment. A subject can be identified as having an increased risk of developing a cancer using methods known in the art. For example, a subject can be determined to have a genetic predisposition to cancer (e.g., a family history of a specific form of cancer) or can be determined to have an increased risk of developing cancer based on exposure to environmental toxins or personal behaviors (e.g., smoking)).

Some embodiments of the methods of treating or reducing the risk of developing cancer further include administering one or more additional pharmaceutical agents to the subject (e.g., pain medications and interleukin-2).

Also provided are methods of administering a composition of one or more of any of the immunostimulatory compositions described herein to a subject (e.g., a human). In some embodiments, the administering can occur in two or more doses. In some embodiments, the subject has a disease or disorder (e.g., previously diagnosed as having a specific disease or disorder). Some embodiments further include selecting a subject having a specific disease or disorder prior to administering the one or more immunostimulatory composition to the subject. In some embodiments, the disease or disorder is selected from the group of: a cancer, an infectious disease, a metabolic disease, a degenerative disease, an autoimmune disease, an inflammatory disease, or an immunological disease. In some embodiments, where the subject has a disease or disorder, the amount of composition administered is effective to reduce the number of symptoms of the disease or disorder experienced by the subject; reduce the severity, frequency, or duration of one or more symptoms of the disease or disorder in the subject; and/or improve the therapeutic outcome in the subject.

### Screening Methods

The disclosure in another aspect provides methods for screening for an antagonist of a TLR. The methods according to this aspect involve the steps of contacting a reference cell expressing a TLR with an effective amount of a composition described herein, in the absence of a candidate antagonist of the TLR, to measure a reference amount of signaling by the TLR; contacting a test cell expressing the TLR with an effective amount of the composition, in the presence of the candidate antagonist of the TLR, to measure a test amount of signaling by the TLR; and determining the candidate antagonist of the TLR is an antagonist of the TLR when the reference amount of signaling exceeds the test amount of signaling. The reference cell and the test cell can each express the TLR naturally or artificially, as described above. In one embodiment the reference cell and the test cell are each cells that are representative of a common population of cells, e.g., PBMC taken from a single donor, or 293HEK cells stably transfected with an expression vector for the TLR. In various specific embodiments the TLR can be chosen from TLR8 or TLR7.

Assays for Measuring Immunostimulatory Effects

The immunostimulatory effect of the immunostimulatory oligonucleotides described herein can be measured using any suitable method, in vitro or in vivo. A basis for such measurement can involve a measurement of cell proliferation; intracellular signaling, specifically including but not limited to TLR signaling; expression of a soluble product, such as a cytokine, chemokine, or antibody; expression of a cell surface marker, such as a cluster of differentiation (CD) antigen; or functional activity, such as apoptosis and NK cell cytotoxicity. Methods for making such types of measurements are well known in the art and can include, without limitation, tritiated thymidine incorporation, enzyme-linked immunosorbent assay (ELISA), radioimmunosassay (RIA), bioassay, fluorescence-activated cell sorting, immunoblot (Western blot) assay, Northern blot assay, terminal deoxynucleotide transferase dUTP nick end labeling (TUNEL) assay, reverse transcriptase-polymerase chain reaction (RT-PCR) assay, and chromium release assay. The measurements can be quantitative or qualitative.

In some embodiments, measurements are made specifically for Th1-like immune response. Such measurements can include measurements of specific cytokines, chemokines, antibody isotypes, and cell activity that are associated with a Th1-like immune response, as described above.

In some embodiments, measurements are made specifically for TLR signaling activity. Such measurements can be direct or indirect, and typically they involve measurement of expression or activity of a gene affected by some component of the intracellular signaling pathway mediated by a TLR.

Nucleotide and amino acid sequences of human and murine TLR8 are known. See, for example, GenBank Accession Nos. AF246971, AF245703, NM_{—}016610, XM_{—}045706, AY035890, NM_{—}133212; and AAF64061, AAF78036, NP_{—}057694, XP_{—}045706, AAK62677, and NP_{—}573475, the contents of all of which are incorporated in their entirety herein by reference.

Nucleotide and amino acid sequences of human and murine TLR7 are known. See, for example, GenBank Accession Nos. AF240467, AF245702, NM_{—}016562, AF334942, NM_{—}133211; and AAF60188, AAF78035, NP_{—}057646, AAL73191, and AAL73192, the contents of all of which are incorporated in their entirety herein by reference. Human TLR7 is reported to be 1049 amino acids long. Murine TLR7 is reported to be 1050 amino acids long. TLR7 polypeptides include an extracellular domain having a leucine-rich repeat region, a transmembrane domain, and an intracellular domain that includes a TIR domain.

### Formulations

In some embodiments, the synthetic nanocarriers can be combined with one or more other adjuvants by admixing in the same vehicle or delivery system. Such adjuvants can include, but are not limited to, mineral salts, such as alum, alum combined with monophosphoryl lipid (MPL) A of Enterobacteriaceae, such as Escherichia coli, Salmonella minnesota, Salmonella typhimurium, or Shigella flexneri or specifically with MPL® (AS04), MPL A of above-mentioned bacteria separately, saponins, such as QS-21, Quil-A, ISCOMs, ISCOMATRIX™, emulsions such as MF59™, Montanide® ISA 51 and ISA 720, AS02 (QS21+squalene+MPL®), liposomes and liposomal formulations such as AS01, synthesized or specifically prepared microparticles and microcarriers such as bacteria-derived outer membrane vesicles (OMV) of Neisseria meningitidis, N. gonorrheae, Francisella novicida and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments. The doses of such other adjuvants can be determined using conventional dose ranging studies.

In some embodiments, the synthetic nanocarriers can be combined with an antigen different, similar, or identical to those coupled to a nanocarrier (with or without adjuvant, utilizing or not utilizing another delivery vehicle) administered separately at a different time-point and/or at a different body location and/or by a different immunization route or with another antigen and/or adjuvant-carrying synthetic nanocarrier administered separately at a different time-point and/or at a different body location and/or by a different immunization route.

Populations of synthetic nanocarriers can be combined to form pharmaceutical dosage forms according to the present disclosure using traditional pharmaceutical mixing methods. These include liquid-liquid mixing in which two or more suspensions, each containing one or more subset of nanocarriers, are directly combined or are brought together via one or more vessels containing diluent. As synthetic nanocarriers can also be produced or stored in a powder form, dry powder-powder mixing could be performed as could the re-suspension of two or more powders in a common media. Depending on the properties of the nanocarriers and their interaction potentials, there can be advantages conferred to one or another route of mixing.

Typical compositions that include synthetic nanocarriers can include inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol).

Compositions according to the disclosure can include synthetic nanocarriers in combination with pharmaceutically acceptable excipients. The compositions can be made using conventional pharmaceutical manufacturing and compounding techniques to arrive at useful dosage forms. Techniques suitable for use in practicing the present disclosure can be found in Handbook of Industrial Mixing: Science and Practice, Edited by Edward L. Paul et al., 2004, John Wiley & Sons, Inc.; and Pharmaceutics: The Science of Dosage Form Design, 2nd Ed. Edited by M. E. Auten, 2001, Churchill Livingstone. In some embodiments, synthetic nanocarriers can be suspended in sterile saline solution for injection together with a preservative.

It is to be understood that the compositions described herein can be made in any suitable manner, and the invention is in no way limited to compositions that can be produced using the methods described herein. Selection of an appropriate method can require attention to the properties of the particular moieties being associated.

In some embodiments, synthetic nanocarriers can be manufactured under sterile conditions or are terminally sterilized. This can ensure that resulting composition are sterile and non-infectious, thus improving safety when compared to non-sterile compositions. This provides a valuable safety measure, especially when subjects receiving synthetic nanocarriers have immune defects, are suffering from infection, and/or are susceptible to infection. In some embodiments, synthetic nanocarriers can be lyophilized and stored in suspension or as lyophilized powder depending on the formulation strategy for extended periods without losing activity.

### Dosing and Administration

The immunostimulatory oligonucleotides described herein can be used alone, in combination with themselves, in combination with another agent, or in combination with themselves and with another agent. In addition to the conjugates described herein, the immunostimulatory oligonucleotide in combination with another agent can also be separate compositions that are used together to achieve a desired effect. For example, an immunostimulatory oligonucleotide and a second agent can be mixed together and administered to a subject or placed in contact with a cell as a combination. As another example, an immunostimulatory oligonucleotide and a second agent can be administered to a subject or placed in contact with a cell at different times. As yet another example, an immunostimulatory oligonucleotide and a second agent can be administered to a subject at different sites of administration.

The immunostimulatory oligonucleotide and/or the antigen and/or other therapeutics can be administered without a delivery vehicle, but with some type of inactive, physiologically acceptable excipient, e.g., purified saline or buffer, such as phosphate buffered saline (PBS), or using any delivery vehicle known in the art. For instance the following delivery vehicles have been described: cochleates (Gould-Fogerite et al., 1994, 1996); emulsomes (Vancott et al., 1998, Lowell et al., 1997); Immune stimulating complexes (ISCOMs) (Mowat et al., 1993, Carlsson et al., 1991, Hu et., 1998, Morein et al., 1999) ((ISCOMs are spherical open cage-like structures (e.g., 40 nm in diameter) that are spontaneously formed when mixing together cholesterol, phospholipids, and saponins under a specific stoichiometry); liposomes (Childers et al., 1999, Michalek et al., 1989, 1992, de Haan 1995a, 1995b); live bacterial vectors (e.g., Salmonella, Escherichia coli, bacille Calmette-Guerin, Shigella, Lactobacillus) (Hone et al., 1996, Pouwels et al., 1998, Chatfield et al., 1993, Stover et al., 1991, Nugent et al., 1998); live viral vectors (e.g., vaccinia, canarypox, fowlpox, adenovirus, herpes simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g., carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); Virosomes (Gluck et al., 1992, Mengiardi et al., 1995, Cryz et al., 1998); and virus-like particles (Jiang et al., 1999, Leibl et al., 1998). Other delivery vehicles are known in the art.

Doses of dosage forms contain varying amounts of populations of synthetic nanocarriers and varying amounts of antigens. The amount of synthetic nanocarriers and/or antigens present in the dosage forms can be varied according to the nature of the antigens, the therapeutic benefit to be accomplished, and other such parameters. In some embodiments, dose ranging studies can be conducted to establish useful and/or preferred therapeutic amounts of the population of synthetic nanocarriers and the amount of antigens to be present in the dosage form. In some embodiments, the synthetic nanocarriers and the antigens are present in the dosage form in an amount effective to generate an immune response to the antigens upon administration to a mammalian subject. It can be possible to determine amounts of the antigens effective to generate an immune response using conventional dose ranging studies and techniques in subjects. Dosage forms can be administered at a variety of frequencies. In a preferred embodiment, at least one administration of the dosage form is sufficient to generate a pharmacologically relevant response. In more preferred embodiments, at least two administrations, at least three administrations, or at least four administrations, of the dosage form are utilized to ensure a pharmacologically relevant response.

The term “effective amount” refers generally to an amount necessary or sufficient to bring about a desired biologic result or outcome. For example, an effective amount can be an amount sufficient to stimulate an immune response (e.g., a Th1-like immune response or a proinflammatory immune response) in a subject (e.g., a human subject). In another example, an effective amount can be an amount sufficient to mediate a decrease in the number of symptoms of a cancer in a subject, an amount sufficient to reduce the severity, frequency, or duration of one or more symptoms of a cancer in a subject, or an amount sufficient to reduce the risk of developing a cancer in a subject. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned that does not cause substantial toxicity and yet is entirely effective to treat the particular subject. The effective amount for any particular application can vary depending on such factors as the disease or condition being treated, the particular oligonucleotide being administered, the size of the subject, or the severity of the disease or condition. One of ordinary skill in the art can empirically determine the effective amount of a particular immunostimulatory oligonucleotide and/or antigen and/or other therapeutic agent without necessitating undue experimentation.

Subject doses of the compounds described herein for systemic or local delivery typically range from about 10 ng to 10 mg (e.g., 10 ng to 8 mg, 10 ng to 5 mg, 500 ng to 5 mg, 1 μg to 5 mg, 500 μg to 5 mg, or 1 mg to 5 mg) per administration, which depending on the application could be given daily, weekly, or monthly and any other amount of time therebetween or as otherwise required. More typically systemic or local doses range from about 1 microgram to 1 milligram per administration, and most typically from about 10 micrograms to 100 micrograms, with 2-4 administrations being spaced days or weeks apart. Higher doses can be required for parenteral administration. In some embodiments, however, parenteral doses for these purposes can be used in a range of 5 to 10,000 times higher than the typical doses described above.

For any compounds and compositions described herein the therapeutically effective amount can be initially determined from animal models. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

For clinical use the immunostimulatory oligonucleotide described herein can be administered alone or formulated as a delivery complex via any suitable route of administration that is effective to achieve the desired therapeutic result. Routes of administration include enteral and parenteral routes of administration. The compositions described herein can be administered by a variety of routes of administration, including but not limited to subcutaneous, intramuscular, intradermal, oral, intranasal, transmucosal, sublingual, rectal, ophthalmic, transdermal, transcutaneous or by a combination of these routes.

### Methods of Making an Immunostimulatory Composition

Also provided are methods of making immunostimulatory compositions. These methods include: (a) isolating at least one 10 to 40 nucleotide sequence from the first 80 bases from a 5′- or 3′-terminus of a positive-sense single-stranded RNA virus genome (e.g., using any of the methods and/or exemplary viral sequences described herein), and/or at least one 10 to 40 nucleotide sequence from the first 80 bases from a 5′-terminus of a negative-sense single-stranded RNA virus genome that has immunostimulatory activity (e.g., using any of the methods and/or exemplary viral sequences described herein); and (b) mixing the at least one isolated 10 to 40 nucleotide sequence from the first 80 bases from a 5′- or 3′-terminus of a positive-sense single-stranded RNA virus genome, and/or the at least one isolated 10 to 40 nucleotide sequence from the first 80 bases from a 5′-terminus of a negative-sense single-stranded RNA virus genome with a carrier and/or a pharmaceutically or physiologically acceptable excipient (e.g., phosphate buffered saline (PBS)). The isolated nucleotide sequence can also be tested using the methods described herein to confirm that it is immunostimulatory.

In some embodiments, the isolated nucleic acid contains one or more of the modifications described herein (e.g., one or more of the base modifications, sugar modifications, and/or backbone modifications described herein). In some embodiments, the nucleic acid is at least partially double-stranded or fully double-stranded. In some embodiments, the at least one isolated nucleic acid contains at least one deoxyribonucleotide. Some embodiments further include adding at least one condensing agent (e.g., a cationic lipid).

In some embodiments, the carrier is a synthetic nanocarrier (e.g., a synthetic nanocarrier that contains at least one biodegradable polymer). Non-limiting examples of synthetic nanocarriers are described herein. Some embodiments further include covalently or non-covalently coupling the at least one isolated nucleic acid to the surface of the synthetic nanocarrier. Some embodiments further include encapsulating the at least one isolated nucleic acid within the synthetic nanocarrier (e.g., using the method described in Example 8). Some embodiments further include the addition of at least one antigen.

### EXAMPLES

The following examples are not meant to limit the inventions described herein, which are defined in the claims.

### Example 1

### Terminal Oligonucleotides from the 3′-End of Chikungunya Togavirus Genome and from the 5′-End of Ebola Filovirus Genome Stimulate Human TLR8

This example demonstrates for the first time that sequences directly taken from the 3′-end of Chikungunya virus (a togavirus, positive-sense, ssRNA virus) or from the 5′-end of Ebola virus (a filovirus, negative-sense, ssRNA virus) activate human TLR8.

A 27-ribonucleotide Chikungunya virus sequence used in this and the following experiments is 5′-GAGAUGUUAUUUUGUUUUUAAUAUUUC-3′ (SEQ ID NO:1). It is designated SL-0001 and comes exactly from the 3′-terminus of virus strain TSI-GSD-218 (GenBank accession number L37661) and corresponds to genomic sequence bases 12010-12036.

The 34-ribonucleotide Ebola virus sequence is 5′-AAGAAGAAAUAGAUUUA UUUUUAAAUUUUUGUGU-3′ (SEQ ID NO:2), henceforth designated SL-0005. This sequence comes from the 5′-terminus of the Zaire strain and directly corresponds to bases 14-47 of viral RNA genome (complementary to bases 18946-18913 of cDNA sequence shown in GenBank entry NC_{—}002549).

Two control RNA sequences, R-0002 and R-0006, have been described earlier (Forsbach et al., 2008, J. Immunol., 180:3729-38). Both of them are capable of activating human TLR8 with R-0006 showing higher activity than R-0002 (Forsbach et al., 2008, J. Immunol., 180:3729-38).

Phosphorothioated SL-0001, SL-0005, R-0002 and R-0006 were synthesized (Sigma-Aldrich, USA) and assayed using human embryonic kidney 293 cell line stably transfected by human TLR8 (InvivoGen, USA). In addition, these cells contain NF-kB-inducible SEAP (secreted embryonic alkaline phosphatase) reporter gene, which enables the enzyme-based detection of human TLR8 activation and are designated HEK-Blue™ hTLR8. Briefly, 10,000-20,000 of these cells were seeded in flat-bottom 96-well plates at 200 μl volumes and 6-16 hours later treated with RNA oligonucleotides complexed with DOTAP (Sigma-Aldrich). DOTAP is N-[1-(2,3-Dioleoyloxy)propyl]-N,N,N-trimethylammonium methyl-sulfate used as a liposomal transfection reagent.

Specifically, serial oligonucleotide dilutions in HBS (HEPES buffered saline) buffer were mixed with equal volumes of 200 μg/ml DOTAP solution and incubated for 15-30 minutes. Resulting complexes were mixed 1:1 with complete RPMI (Roswell Park Memorial Institute) medium (10% FBS) and 100 μl of this mixture was used to replace the same volume of RPMI from HEK-Blue™ hTLR8-containing microplate wells.

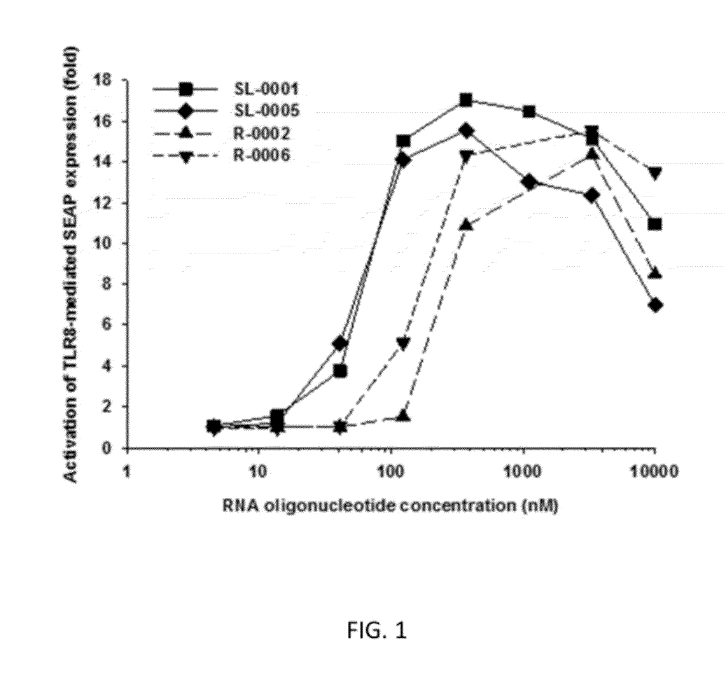

TLR8 activation was monitored at 18-42 hours post transfection using the SEAP detection media QUANTI-Blue™ (InvivoGen) with 20 μl of cell supernatant added to 200 μl of detection media and absorbance at 0.5-3 hour interval measured at 655 nm Results of such an experiment are shown in FIG. 1 with activation presented as fold increase of SEAP induction over the background (medium from cells treated with DOTAP only). Both control ribonucleotides, R-0002 and R-0006, induced TLR8-driven activation of SEAP expression at concentrations and levels similar to earlier reported (Forsbach et al., 2008, J. Immunol., 180:3729-38). At the same time, both novel oligonucleotides, SL-0001 and SL-0005, have shown stronger levels of TLR8 activation, which was detected starting at 40 nM concentrations and reached plateau at 100-300 nM range (FIG. 1). No activation of SEAP expression was seen when the parental control cell line (HEK-Blue™ Null) or similar cell lines, expressing human TLR3 and TLR9 (HEK-Blue™ hTLR3 and HEK-Blue™ hTLR9) were used.

### Example 2

### Terminal Oligonucleotides from the 3′-End of Chikungunya Togavirus Genome and from the 5′-End of Ebola Filovirus Genome Induce TNF-α in Murine Macrophages

TNF-α is an early pro-inflammatory cytokine, which is rapidly induced during the acute phase of innate immune response. Induction of TNF-α by macrophages is a hallmark of human TLR8 activation (Forsbach et al., 2008, J. Immunol., 180:3729-38; Ghosh et al., 2007, Int. Immunopharmacol., 7:1111-21; Gorden et al., 2005, J. Immunol., 174:1259-68). At the same time, TLR8 gene is inactive in mice, while TLR7 is functional (Jurk et al., 2002, Nat. Immunol., 3:499). Therefore, induction of TNF-α by ssRNA oligonucleotides in mouse cells suggests TLR7-mediated activation as demonstrated for R-0006 known to be capable of activating both TLR7 and TLR8 (Forsbach et al., 2008, J. Immunol., 180:3729-38). In the following experiment mouse macrophage cell line, J774, has been transfected by test ribonucleotides as described above for HEK-Blue™ hTLR8 cells (with the exception that 2-10×10⁴ cells/well were used) and TNF-α was measured in culture supernatants at 16-20 hours after transfection.

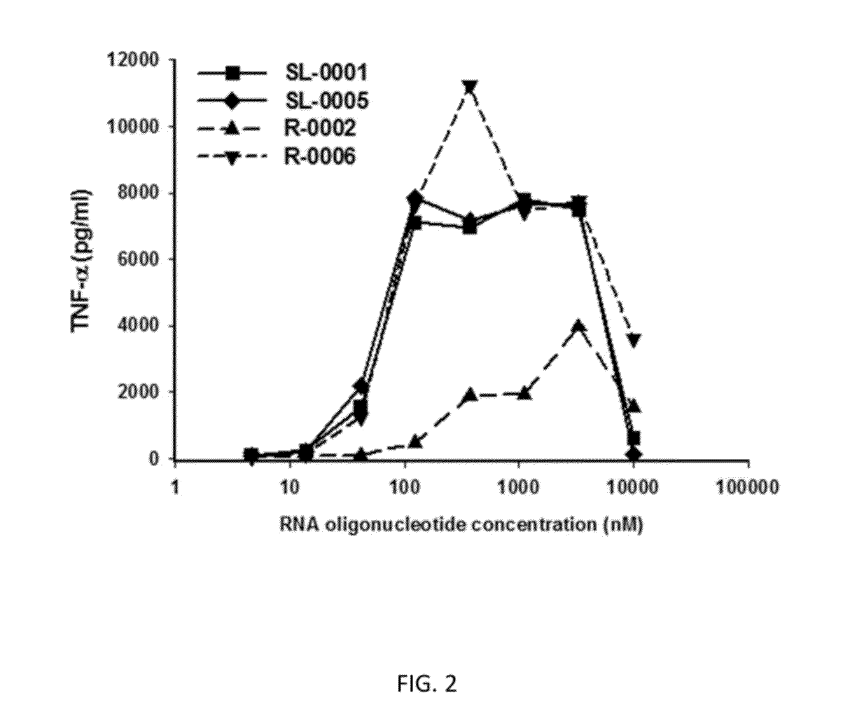

As seen in FIG. 2, both SL-0001 and SL-0005 strongly induced TNF-α expression, to the level seen when R-0006 was used (starting in 10-15 nM interval with a plateau at 100-150 nM). TNF-α induction by R-0002 was weaker starting at 10-fold higher concentrations and never reaching the same levels as SL-0001, SL-0005 and R-0006. Notably, it is known that R-0002 is an inferior TLR7 agonist compared to R-0006 (Forsbach et al., 2008, J. Immunol., 180:3729-38).

Collectively, we have shown that oligoribonucleotide sequences SL-0001 and SL-0005 directly derived from the 3′-terminus of a positive-sense single-stranded RNA virus or from the 5′-terminus of a negative-sense single-stranded RNA virus, are capable of activating TLR7 and TLR8 in mouse and human cells, respectively. Thus, both SL-0001 and SL-0005 could be used for TLR7/8-driven immune stimulation. While TLR7 and 8 are known sensors of ssRNA, no specific sequences that are capable of their activation have been described earlier for positive-sense ssRNA viruses or for 5′-termini of negative-sense ssRNA viruses.

### Example 3

### Oligonucleotides Derived from Genomic 5′-Ends of Flaviviruses, Japanese Encephalitis Virus, and of Murray Valley Encephalitis Virus Stimulate Human TLR8

Experiments were performed to determine whether sequences from the 5′-ends of Japanese encephalitis virus (a flavivirus, positive-sense, ssRNA virus) or from the 5′-end of Murray valley encephalitis virus (another flavivirus) would activate human TLR8.

The following sequences were used in these experiments. 33-ribonucleotide SL-0004, 5′-GUUUAUCUGUGUGAACGAUAGUGCAGUUUAAAC-3′ (SEQ ID NO:3), was derived from the 5′-terminus of Japanese encephalitis virus (GenBank accession number NC_{—}001437) and corresponds to nucleotides 5-20, 45-46 and 58-71. Sequences SL-0017, SL-0018, SL-0019 and SL-0020 (SEQ ID NO:4-7) were derived from SL-0004 and are referenced below according to their alignment with SL-0004. Dashes within the sequences signify deleted nucleotides.

(SEQ ID NO: 3)

| | |
|---|---|
| SL-0004 | 5′-GUUUAUCUGUGUGAACGAUAGUGCAGUUUAAAC-3′ |

(SEQ ID NO: 4)

| | |
|---|---|
| SL-0017 | 5′-GUUUAUCUGUGUGAACGAUAGU-3′ |

(SEQ ID NO: 5)

| | |
|---|---|
| SL-0018 | 5′-GUUUAUCUGUGUG-3′ |

(SEQ ID NO: 6)

| | |
|---|---|
| SL-0019 | 5′-CUGUGUGAACGAUAGUGCAG-3′ |

(SEQ ID NO: 7)

| | |
|---|---|
| SL-0020 | 5′-GUUUAUCUGUGUG----------CAGUUUAAAC-3′ |

26-ribonucleotide SL-0002, 5′-UUUUUUGGAGCUUUUGAUUUCAAAUG-3′ (SEQ ID NO:8), was from the exact 5′-terminus of Murray valley encephalitis virus (GenBank accession number NC000943), and directly corresponds to nucleotides 73-98. Sequences SL-0015 and SL-0016 (SEQ ID NO:9 and SEQ ID NO:10) are derived from SL-0002 and are referenced below according to their alignment with SL-0002. They directly correspond to nucleotides 73-92 and 76-95 of the Murray Valley encephalitis genome.

| (SEQ ID NO: 8) | |
|---|---|
| SL-0002 | 5′-UUUUUUGGAGCUUUUGAUUUCAAAUG-3′ |
| (SEQ ID NO: 9) | |
| SL-0015 | 5′-UUUUUUGGAGCUUUUGAUUU-3′ |
| (SEQ ID NO: 10) | |
| SL-0016 | 5′-UUUGGAGCUUUUGAUUUCAA-3′ |

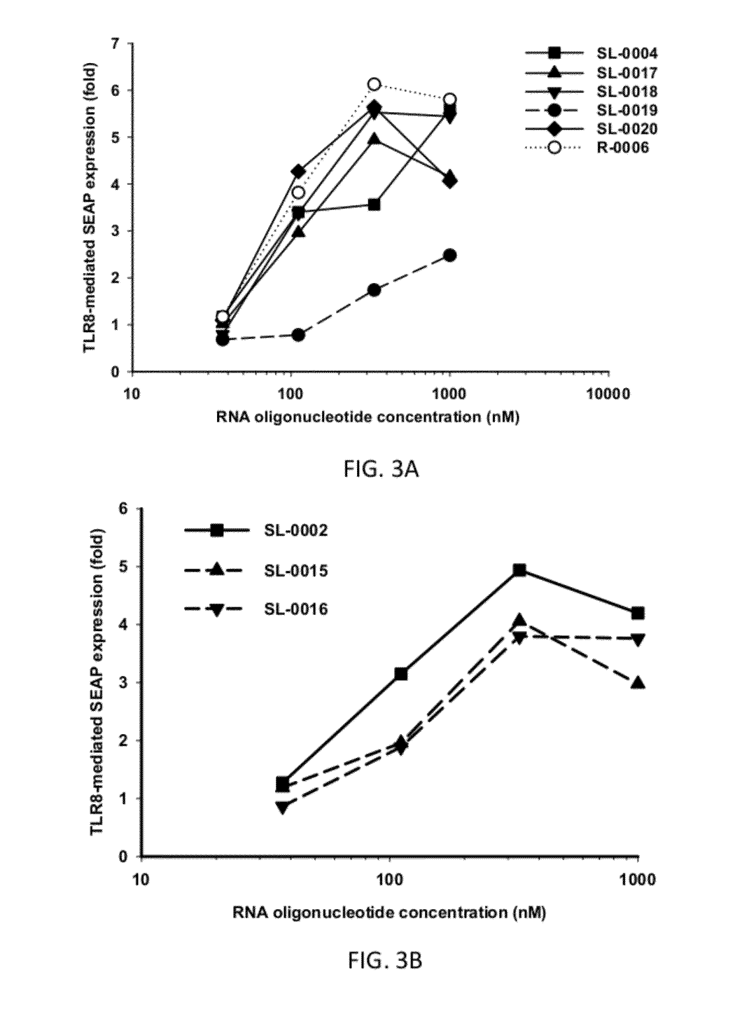

Phosphorothioated SL-0004, SL-0017, SL-00018, SL-0019, SL-0020, SL-0002, SL-0015, and SL-0016 were synthesized (Sigma-Aldrich, USA) and assayed using human embryonic kidney 293 cell line stably transfected by human TLR8 (InvivoGen, USA), as described above in Example 1. TLR8 activation was monitored at 40 hours post transfection as described in Example 1. The results for sequences SL-0004, SL-0017, SL-00018, SL-0019, and SL-0020 are shown in FIG. 3A, and the results for sequences SL-0002, SL-0015, and SL-0016 are shown in FIG. 3B. The control oligonucleotide R-0006 (described above) was also used in the assay shown in FIG. 3A.

All of the tested oligonucleotides, with the exception of SL-0019, induce strong levels of TLR8 activation that are at least comparable with R-0006. TLR8 activation was detected starting at a concentration of 40 nM, and reached plateau in the 100-300 nM range (FIGS. 3A and 3B). Moreover, the central 10 bases of SL-0004, but not its 5′-terminal six bases seem to be dispensable for this activity, since the 23-nucleotide sequence SL-0020 demonstrated activity similar or exceeding that of SL-0004, while the activity of SL-0019 was markedly weaker (FIG. 3A).

No activation of SEAP expression was seen when the parental control cell line (HEK-Blue™ Null) or similar cell lines expressing human TLR3 and TLR9 (HEK-Blue™ hTLR3 and HEK-Blue™ hTLR9) were used.

This example demonstrates that sequences from the 5′-ends of Japanese encephalitis virus or Murray valley encephalitis virus activate human TLR8.

### Example 4

### Terminal Oligonucleotides Derived from the Genomic 5′-End of Flavivirus Japanese Encephalitis Virus Genome Induce TNF-α Production in Murine Macrophages

Experiments were performed to determine whether sequences from the 5′-end of Japanese encephalitis virus (a flavivirus, positive-sense, ssRNA virus) would stimulate production of inflammatory cytokine TNF-α from mouse macrophages.

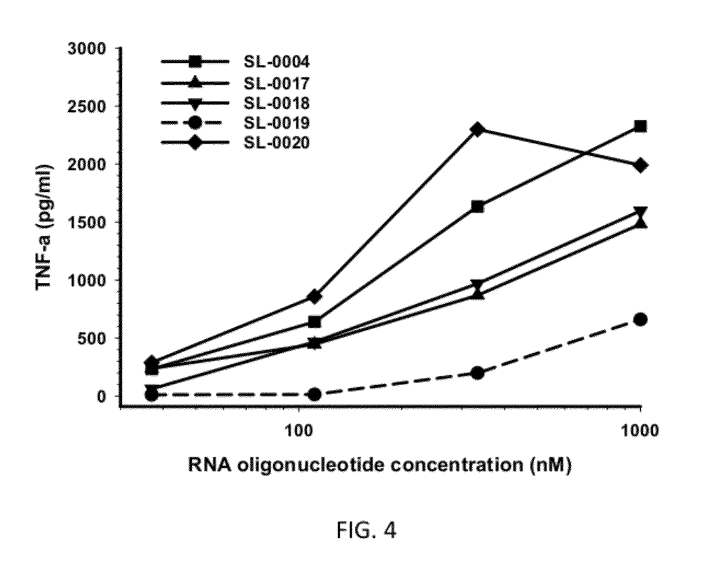

Phosphorothioated oligonucleotides SL-0004, SL-0017, SL-00018, SL-0019, and SL-0020 (described above) were tested in the mouse macrophage cell line J774. The cultured cells were transfected by the test ribonucleotides as described above in Example 2. All of the test oligonucleotides show strong induction of TNF-α expression at 24 hours post-transfection (although induction by SL-0019 was weaker than the level of induction observed for the other oligonucleotides). Induction of TNF-α was observed at nucleotide concentrations of 40 nM, and for the most potent SL-0020, reached plateau at 300 nM (FIG. 4). Notably, the relative levels of TNF-α induction corresponded to the levels of TLR8 activation observed in the human TLR8 activation assay (FIG. 3A).

This example demonstrates that sequences from the 5′-ends of Japanese encephalitis virus or Murray valley encephalitis virus induce TNF-α production in murine macrophages.

### Example 5

### Series of Related Oligonucleotides Derived from the Viral Genome Termini of Chikungunya Togavirus (3′-End) and Ebola Filovirus (5′-End) Stimulate Human TLR8

This example demonstrates that ribonucleotide sequences related to SL-0001 (SEQ ID NO:1) or to SL-0005 (SEQ ID NO:2) described earlier in Example 1, activate human TLR8 and that this activity is related to the integrity of U-rich domains contained in both of these sequences.

The SL-0001 sequence is from the Chikungunya virus genome and the SL-0005 sequence is from the Ebola virus genome (described in detail above). The sequences SL-0011, SL-0012, SL-0013, and SL-0014 (SEQ ID NO:11-14) are taken directly from within SL-0001 (and thus, from Chikungunya virus genome), and their alignment is shown below.

| (SEQ ID NO: 1) | |
|---|---|
| SL1 | 5′-GAGAUGUUAUUUUGUUUUUAAUAUUUC-3′ |
| (SEQ ID NO: 11) | |
| SL11 | 5′-UUAUUUUGUUUUUAAUAUUUC-3′ |
| (SEQ ID NO: 12) | |
| SL12 | 5′-UUAUUUUGUUUUUAAUAUUU-3′ |
| (SEQ ID NO: 13) | |
| SL13 | 5′-UGUUAUUUUGUUUUUAAUAU-3′ |
| (SEQ ID NO: 14) | |
| SL14 | 5′-AGAUGUUA--UUGUUU-AAUAUUU-3′ |

The sequences SL-0021, SL-0022, SL-0023, SL-0024, and SL-0025 (SEQ ID NO:15-19) were taken directly from within SL-0005 or from adjacent genomic sequences of Ebola virus, and their alignment is shown below. Specifically, those sequences extending beyond SL-0005 correspond to the following nucleotide positions in Ebola virus Zaire RNA genomic sequence: SL-0022 (SEQ ID NO:16), nucleotides 8-18 and 35-45 (complementary to bases 18952-18842 and 18925-18915 of cDNA sequence shown in GenBank entry NC_{—}002549); SL-0024 (SEQ ID NO:18), nucleotides 6-17 and 39-47 (complementary to bases 18954-18843 and 18921-18913 of cDNA sequence shown in GenBank entry NC_{—}002549); SL-0025 (SEQ ID NO:19), nucleotides 1-19 (exact 5′-end of virus genome, complementary to bases 18959-18941 of cDNA sequence shown in GenBank entry NC_{—}002549). The sequences SL-0021 (SEQ ID NO:15) and SL-0023 (SEQ ID NO:17) are completely contained within SL-0005. Dashes within the sequences below signify deleted nucleotides.

| SEQ ID NO: | | |
|---|---|---|
| SL-0005 | 5′-AAGAAGAAAUAGAUUUAUUUUUAAAUUUUUGUGU-3′ | (2) |
| SL-0021 | 5′-AGAAAUAGAUUUAUUUUU-3′ | (15) |
| SL-0022 | 5′-ACAAAAAAGAA----------------UAAAUUUUUGU-3′ | (16) |
| SL-0023 | 5′-AUUUAUUUUUAAAUUUUUGUGU-3′ | (17) |
| SL-0024 | 5′-ACACAAAAAAGA---------------------UUUUUGUGU-3′ | (18) |
| SL-0025 | 5′-UGGACACACAAAAAAGAAG-3′ | (19) |

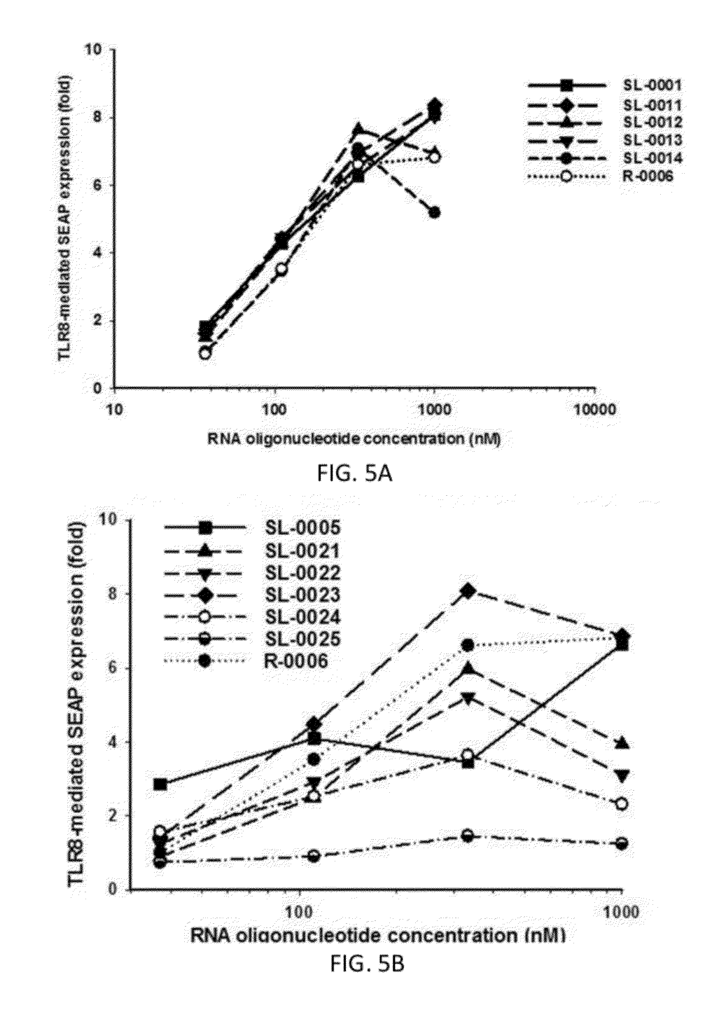

Phosphorothioated SL-0001, SL-0011, SL-0012, SL-0013, SL-0014, SL-0005, SL-0021, SL-0022, SL-0023, SL-0024, SL-0025, and control sequence R-0006 were synthesized (Sigma-Aldrich, USA) and assayed as described above using human embryonic kidney 293 cell line stably transfected by human TLR8 (InvivoGen, USA). TLR8 activation was monitored at 40 hours post-transfection. The data for SL-0001 and related sequences are shown in FIG. 5A, and the data for SL-0005 and related sequences are shown in FIG. 5B (SL-0005 and related sequences), with activation presented as fold-increase of SEAP induction over the background (medium from cells treated with DOTAP only). All of the SL-0001-related oligonucleotides, directly taken from Chikungunya virus genome, have equal or stronger levels of TLR8 activation than the control R-0006 oligonucleotide, with an effect detected at 40 nM concentrations and reaching a plateau in the 100-300 nM range (FIG. 5A).

All of the SL-0005-related oligonucleotides taken from Ebola virus genome (with the exception of SL-0025, which does not possess any U-rich domains present in other related sequences), have equal or stronger levels of TLR8 activation than the control R-0006 oligonucleotide, with an effect detected at 40 nM concentrations and reaching a plateau in the 100-300 nM range (FIG. 5B). Of these, the sequence SL-0023 exhibited even stronger activity than the parental SL-0005 sequence (and an activity higher than the control R-0006 sequence) (see, FIG. 5B).

No activation of SEAP expression was observed when the parental control cell line (HEK-Blue™ Null) or similar cell lines expressing human TLR3 and TLR9 (HEK-Blue™ hTLR3 and HEK-Blue™ hTLR9) were used.

This example demonstrates that sequences from the 3′ terminus of Chikungunya togavirus and the 5′-terminus of Ebola filovirus stimulate human TLR8.

### Example 6

### Terminal Oligonucleotides from the 3′-End of Chikungunya Togavirus Genome and from the 5′-End of Ebola Filovirus Genome Induce TNF-α, IL-6, IFN-γ, and IL-12 (p40) in Murine Splenocytes and Induce Activation of Diverse Immune Cells

This example demonstrates that ribonucleotide sequences from the 3′-end of Chikungunya togavirus genome and from 5′-end of Ebola filovirus genome induce immune cell activation and production of pro-inflammatory and immune cytokines in primary mouse splenocyte cultures in vitro.

Primary murine splenocyte cultures are known to exhibit characteristics similar to cellular reactions in vivo, especially if assayed immediately after in vitro culture. The SL-0001, SL-0011, SL-0012, and SL-0014 oligonucleotides (derived directly from Chikungunya virus genome 3′-end) (described above), and the SL-0005, SL-0021, and SL-0023 oligonucleotides (derived from Ebola virus genome 5′-end) (described above), were complexed with DOTAP (as described above) and used to treat fresh (overnight) murine splenocytes (2 separate cultures from individual animals; in RPMI with 10% FBS), which were meshed, counted, and plated at 10⁶ cells/well. The following controls were used: intact cells, DOTAP-treated (mock) cells, and cells treated with TLR7/8 agonist R848 (1 μM). All of the oligonucleotides were used at 200 nM. Cytokine production in the culture supernatants was measured after overnight incubation by ELISA (BD Biosciences) according to manufacturer's recommendation.

In addition, upon removal of the supernatant at 20 hours post-incubation, the mouse cells were stained for the following cell markers: GR1, F4/80, CD11c, CD220, CD3, Ly49b, and CD69, and analyzed by FACS. Different immune cell populations were distinguished as follows: macrophages (F4/80⁺/GR1⁻); plasmacytoid dendritic cells or pDC (CD11c⁺/CD220⁺); B cells (CD220⁺/CD11c⁻); NK (CD3⁻/Ly49b⁺); myeloid DC or mDC (CD11c⁺/CD220⁺); granulocytes (eosinophils) (GR1^{+high}/F4/80⁻); T cells (CD3⁺); and NKT cells (CD3⁺/Ly49b⁺). All immune cell populations were quantified by the expression of CD69 (am early activation marker).

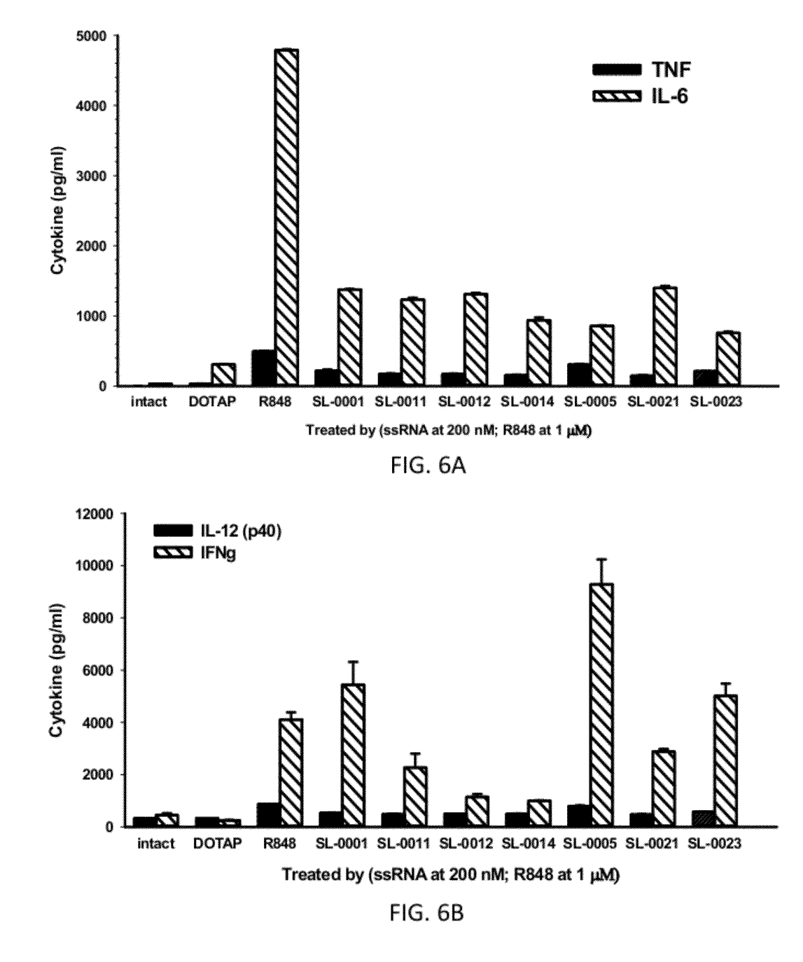

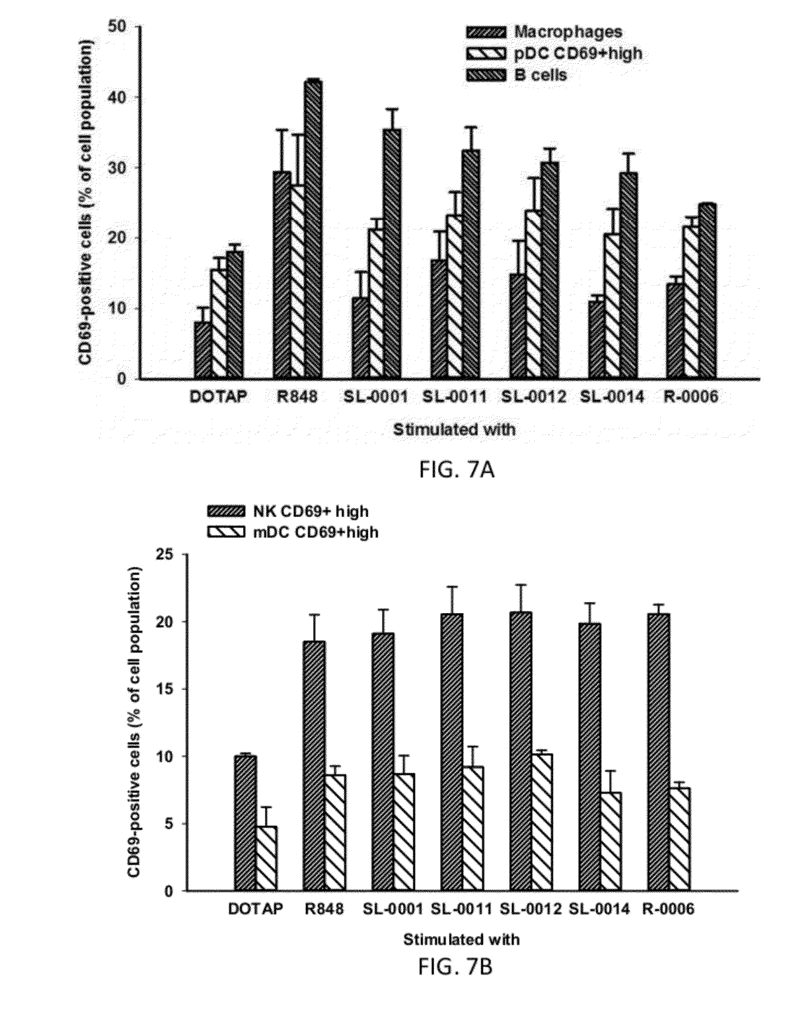

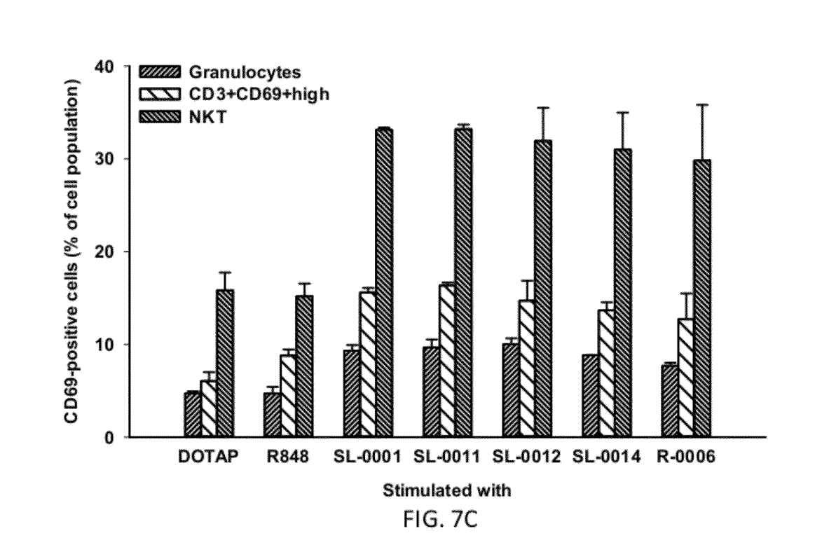

The cytokine expression/secretion induced by SL-0001, SL-0011, SL-0012, SL-0014, SL-0005, SL-0021, and SL-0023 are shown in FIGS. 6A and 6B. Notably, while all of the tested oligonucleotides induced substantial levels of inflammatory cytokines TNF-α and IL-6, these were lower than the levels induced by the TLR7/8 agonist, R848 (FIG. 6A). While known to be important markers of early immune responses, pro-inflammatory cytokines are sometimes associated with induction of flu-like symptoms and other similar systemic side-effects. At the same time, several of the oligonucleotides tested (SL-0001, SL-0005, and SL-0023) induced similar or higher levels (compared with higher concentrations of R848) of immune cytokines IFN-γ and IL-12, known to be instrumental in the generation of specific immune responses (FIG. 6B). Similarly, all of sequences derived from the 3′-end of Chikungunya virus (SL-0001, SL-0011, SL-0012, and SL-0014) induced activation in most of the tested immune cell populations (FIG. 7A-C). This activation was at least comparable to that generated by treatment with 1 μM of R848, and usually exceeded the effects exhibited by the control oligonucleotide R-0006 (FIG. 7A-C). Macrophage activation by SL-0001, SL-0011, SL-0012, or SL-0014 and B-cell activation by SL-011 was especially higher than the activation observed for R-0006 (FIG. 7A), while granulocyte, T cell, and NKT cell activation by SL-0001, SL-0011, SL-0012, or SL-0014 exceeded the activation observed for R848 (FIG. 7C).

This example demonstrates that sequences from the 3′-terminus of Chikungunya togavirus genome and the 5′-terminus of Ebola filovirus genome induce production of TNF-α, IL-6, IFN-γ, and IL-12 (p40) in murine splenocytes, and induce activation of diverse immune cell populations, including macrophages, B-cells, granulocytes, T cells, and NKT cells.

### Example 7

### Terminal Oligonucleotides from 3′-End of Chikungunya Togavirus Genome and from 5′-End of Ebola Filovirus Genome Induce Inflammatory and Immune Cytokines in Human Primary Lymphocyte Cultures

This example demonstrates that ribonucleotide sequences from the 3′-end of Chikungunya togavirus genome and from the 5′-end of Ebola filovirus genome induce the production of pro-inflammatory and immune cytokines in primary human donor lymphocyte cultures in vitro.

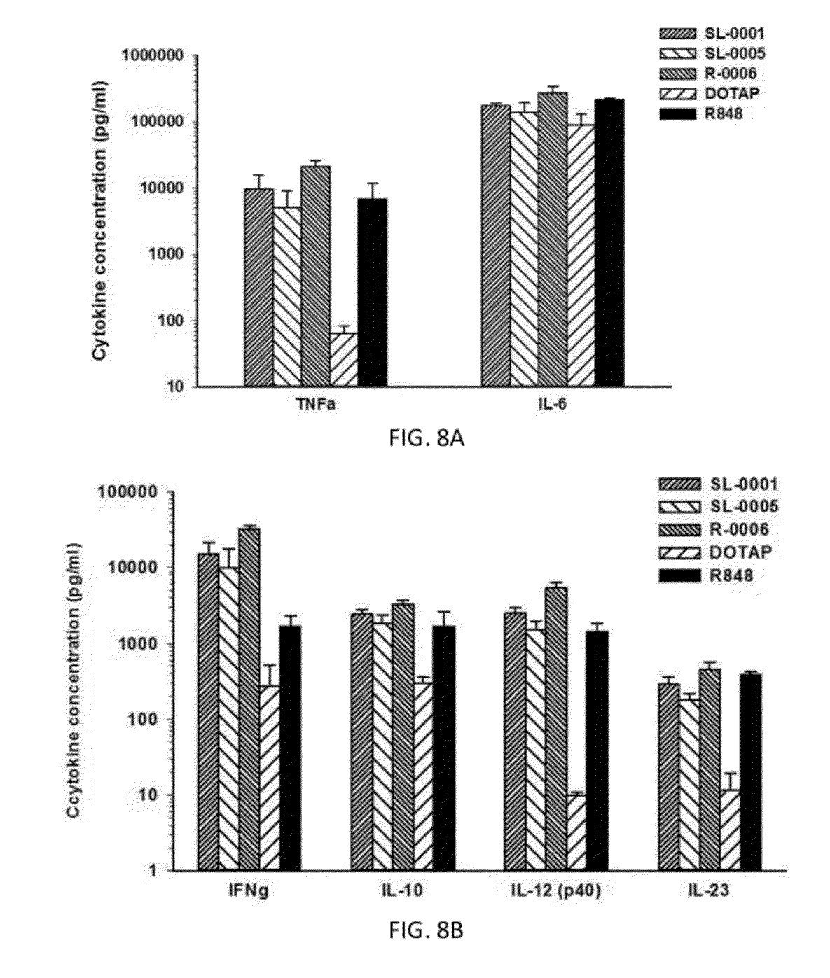

TLR7 and 8 expression and function are partially different in mice and humans (Demaria et al., 2010, J. Clin. Invest., 120(10):3651-3662). Experiments were performed to test cytokine induction by the selected viral oligonucleotides (earlier determined to be immunostimulatory in mouse systems) in cultures from human donors. Lymphocytes from two donors were Ficoll-purified, plated (5×10⁶ cells/well; RPMI, 10% FBS), and treated in duplicate with DOTAP only, R848 (1 μM), or 200 nM of DOTAP-complexed oligoribonucleotides SL-0001 (Chikungunya virus genome 3′-end), SL-0005 (Ebola virus genome 5′-end), or R-0006 (control) (as described in Example 6). Supernatants were analyzed for cytokine expression at 20 hours after incubation by Luminex assays (Aushon BioSystems, Billerica, Mass.). Both SL-0001 and SL-0005 induced significant levels of the pro-inflammatory cytokines TNFα and IL-6 (FIG. 8A), as well as Th1 immune cytokines IFNγ, IL-10, 11-12(p40), and IL-23 (FIG. 8B). The induction of Th2-type cytokines (IL-4 and IL-5) was insignificant. Therefore, both SL-0001 and SL-0005 exhibit significant immunostimulatory activity in humans.

### Example 8

### Immunostimulatory Composition Containing a Synthetic Nanocarrier

Materials

ssRNA: RPS-SEL-PI01, an oligodeoxynucleotide with a phosphorothioate backbone and a nucleotide sequence of 5′-GAGAUGUUAUUUUGUUUUUAAU AUUUC-3′ (SEQ ID NO: 1)(3′-terminus of Chikungunya virus strain TSI-GSD-218 (corresponding to genomic sequence bases 12010-12036)), with a sodium counter-ion, is purchased from Oligo Factory (Holliston, Mass.).

Poly(lactic-co-glycolic acid) (PLGA) with a lactide:glycolide ratio of 54:56, a molecular weight of 25 kDa, a polydispersity index (PDI) of 1.8, and an inherent viscosity of 0.24 dL/g is purchased from SurModics Pharmaceuticals (Product Code 5050 DLG 2.5A) (Birmingham, Ala.).

Poly(lactic acid)-polyethylene glycol (PLA-PEG) block co-polymer with a monomethyl ether PEG block of approximately 5,000 Da and a PLA block of approximately 17,000 Da is synthesized by Selecta Biosciences (Watertown, Mass.).

PLGA with a lactide:glycolide ratio of 76:24, a molecular weight of 106 kDa, a PDI 1.6, and an inherent viscosity of 0.69 dL/g is purchased from SurModics Pharmaceuticals (Product Code 7525 DLG 7A) (Birmingham, Ala.).

Polyvinyl alcohol (MW of 11,000-31,000; 87-89% hydrolyzed) is purchased from J. T. Baker (Part Number U232-08).

Solutions

The following solutions are prepared as described below.

Solution 1 (RPS-SEL-PI01): The oligodeoxynucleotide is prepared at room temperature by dissolving it in a 150 mM KCl/distilled water to achieve a final concentration of 40 mg/mL.

Solution 2: A 100 mg/mL solution of PLGA 5050 DLG 2.5A (described above) in methylene chloride. The solution is prepared by dissolving PLGA 5050 DLG 2.5A in pure methylene chloride.

Solution 3: A 100 mg/mL solution of PLGA 7525 DLG 7A (described above) in methylene chloride. The solution is prepared by dissolving PLGA in pure methylene chloride.

Solution 4: A 100 mg/mL solution of PLA-PEG (described above) in methylene chloride. The solution is prepared by dissolving PLA-PEG in pure methylene chloride.

Solution 5: A 50 mg/mL solution of polyvinyl alcohol (described above) in 100 mM phosphate buffer, pH 8.0.

Steps

A primary water-in-oil emulsion is prepared first. A primary emulsion (W1/O1) is prepared by combining Solution 1 (0.25 mL) (described above), Solution 2 (0.25 mL) (described above), Solution 3 (0.50 mL) (described above), and Solution 4 (0.25 mL) (described above) in a small pressure tube, and sonicating the mixture at 50% amplitude for 40 seconds using a Branson Digital Sonifier® 250. A secondary emulsion (W1/O1/W2) is then prepared by combining solution 5 (2.0 mL) (described above) with the primary W1/O1 emulsion (described above), vortexing for 10 s, and sonicating at 30% amplitude for 60 seconds using the Branson Digital Sonifier® 250.

The W1/O1/W2 emulsion is then added to a beaker containing 70 mM phosphate buffer solution, pH 8.0 (30 mL), and stirred at room temperature for 2 hours to allow the methylene chloride to evaporate and the nanocarriers to form. A portion of the nanocarriers is washed by: transferring the nanocarrier suspension to a centrifuge tube, performing centrifugation at 75,600 rcf at 4° C. for 35 minutes, removing the supernatant, and re-suspending the pellet in phosphate buffered saline. The washing procedure is repeated, and the pellet then re-suspended in phosphate buffered saline for a final nanocarrier dispersion of about 10 mg/mL.

### Example 9

### Nanocarrier-Complexed Terminal Oligonucleotide from the 3′-End of Chikungunya Togavirus Genome in a Mouse Tumor Model

Several immune stimulators (also known as adjuvants) demonstrate the potency to augment immune responses in vivo. One of the most demonstrative correlates of such a response in the area of tumor immunology is the ability to induce anti-cancer immune activity and suppression of the growth of tumor cells in a therapeutic or preventative setting. For this activity to be specific, an immune modulator needs to be co-delivered with the tumor-associated antigen (e.g., an antigen encapsulated into a nanocarrier (NC)).

Thus a nanocarrier-complexed ribonucleotide sequence from 3′-end of Chikungunya togavirus genome is tested to determine whether it augments anti-tumor responses in a mouse cancer model to delay and/or suppress tumor progression. Experiments are performed using a standard mouse tumorigenic model cell line EG.7-OVA. This cell line is syngeneic with C57BL/6 mice with the exception of highly immunogenic ovalbumin protein, which is engineered to be expressed by this cell line. Despite this expression (and the resulting immune response to ovalbumin upon EG.7-OVA cell injection), intact animals usually exhibit tumor progression in ≧90% cases and eventually succumb to the tumor within 3-4 weeks. This progression may be delayed by immunization with ovalbumin, especially in combination with potent adjuvants.

In these experiments, C57BL/6 mice (5/group; 2 separate experiments) are inoculated (s.c., subscapular) with 0.2×10⁶ EG.7-OVA cells. Therapeutic treatment begins at day 3 after the cancer cell inoculation and includes five injections (at days 3, 5, 7, 14, and 21) of NC-encapsulated ovalbumin (OVA) (100 μg NC with OVA load of 4.1%), either in combination with free TLR7/8 agonist R848 or with the NC-complexed oligonucleotide SL-0001 (300 μg of NC-complexed oligonucleotide composition described in Example 8 (60 μL of a 5 mg/mL solution). Injections of phosphate buffered saline (PBS) serve as controls.

The animals are examined over a time of 3 to 10 weeks to determine whether, and if so, when, tumors develop. Animals injected with the compositions described herein should have a significantly delayed onset of tumor development compared to animals injected with PBS. In addition, animals with an existing tumor can show remission of the tumor.

### Example 10

### Nanocarrier-Complexed Immunostimulatory Nucleic Acid in a Ferret Influenza Infection Model

A nanocarrier-complexed ribonucleotide sequence from 3′-end of Chikungunya togavirus genome is tested to determine whether it will afford improved protection against influenza infection in a ferret influenza infection model.

These experiments are performed on outbred, 6- to 10-month old ferrets. During the experiment, the ferrets are housed in a class II isolation facility with free access to food and water. Prior to vaccinations, the animals are confirmed to be seronegative for circulating influenza A (H1N1 and H3N2), and influenza B viruses by heamagglutination inhibition assay (HAI) and enzyme-linked immunosorbent assay (ELISA).

The animals are intramuscularly administered (in the hind leg) two doses (at week 0 and week 2) of: Vaxigrip (80 μL, containing 2.5 μg of hemagglutinin (HA) from each of A/New Calcdonia/20/99 (H1N1), A/New York/55/2004 (H3N2), B/Jiangsu/10/2003, A/Brisbane/59/2007 (H1N1), A/Brisbane/10/2007 (H3N2), and B/Florida/4/2006; Sanofi-Pasteur) in combination with NC-complexed oligonucleotide SL-0001 (300 μg of NC-complexed oligonucleotide composition described in Example 8 (60 μL, of a 5 mg/mL solution), a dose of the Vaxigrip (80 μL, containing 2.5 μg of each HA, described earlier), or a similar volume of PBS.

Six weeks after the first vaccination, all of the animals are inoculated intranasally with 10⁷ TCID₅₀ of A/New Calcdonia/20/99 (H1N1) produced in eggs. During the challenge, nasal washes are taken daily from day 0 to day 6 post-infection. Blood samples are taken from the cranial vena cava at day 3, 5, 7, and 10 post-infection.

Viral secretions in the challenged ferrets are studied by performing nasal washes. These washes are performed using a pipette to apply 1 mL of PBS into the nostrils of each ferret. Subsequently, the animals sneeze and the expelled material (nasal wash sample) is collected and stored at −80° C.

The concentration of viral RNA is measured by real time RT-PCR using primers and probe from the matrix gene of the virus used to challenge the ferrets. RNA is extracted from the samples using the total nucleic acid kit on the semi-automatic Magnapure extraction machine from Roche (Hvidovre, Denmark). The eluted RNA is analyzed in one-step RT-PCR using the RT-PCR One-step kit from Qiagen (Copenhagen, Denmark). The real-time PCR assays are performed on an MX3005 thermocycler from Stratagene (LaJolla, Calif.). For every assay, a standard curve with titrated A/New Calcdonia/99 (H1N1) or A/Brisbane/59/2007 (H1N1) influenza virus are used calculate the relative amount of viral RNA present in the sample.

Influenza-specific IgG and IgA are also assessed in the challenged ferrets. For IgG titration, Maxisorp plates (NUNC, Roskilde, Denmark) are coated at 4° C. overnight with Vaxigrip (1 μg hemagglutinin from H1N1 per mL) in carbonate buffer, pH 9.6. The plates are washed three times with PBS containing 0.05% Tween-20 and blocked with PBS with 1% BSA for two hours. The plates are then washed and 100 μL of serial dilutions of serum sample are tested in duplicate. After a one-hour incubation period followed by washes, 150 μL of biotinylated polyclonal rabbit anti-mink IgG antibody, diluted 1:500 are added and the plates incubated for one hour. After thorough washing, 100 μl of HRP-streptavidin (Dako, Denmark) are added followed by incubation for 30 minutes at room temperature and subsequent development of the reaction with OPD tablets (1,2-phenylendiamin-dihydrochlorid, Dako, Denmark) following the manufacturer's instructions. IgA levels in nasal washes are investigated in a similar fashion as for the above mentioned IgG ELISA, except that a HRP-conjugated anti-dog IgA (AbD-Serotec, Denmark) polyclonal antibody are used instead of the anti-IgG.

The activity of peripheral blood leukocytes from the challenged ferrets are also analyzed in the challenged ferrets. In these experiments, staining is performed as described in Pedersen et al., Veterinary Immunol. Immunopathol. 88:111-122, 2002, with the following modifications. Approximately 2 million peripheral blood leucocytes (PBLs) prepared after hypotonic lysis of erythrocytes with 0.15 M NH₄Cl are cultured in 1 mL of modified RPMI containing 20 mM Hepes and L-Glutamine (Sigma, St. Louis, USA), 10% FCS (fetal calf serum), 100 IU/mL penicillin, and 100 μg/mL streptomycin. For non-specific stimulation of lymphocytes, the cultures are incubated for 4 hours with a medium containing brefeldin A (Sigma, St. Louis, USA) to a final concentration of 10 μg/mL culture, ionomycin (Sigma, St. Louis, USA) to a final concentration of 1 μg/mL, and phorbol-12-myristate-13-acetate (PMA, Sigma, St. Louis, USA) to a final concentration of 20 ng/mL. For the antigen-specific stimulation, the PBLs are cultured 24 hours with medium containing 1 μg/mL of recombinant H1 hemagglutinin from A/New Calcdonia/20/99 (Protein Sciences Corporation, CT, USA).

After culture, the PBLs are fixed in 4% paraformaldehyde and permeabilized with 0.1% saponin (Sigma, St. Louis, USA) and stained with 15 μL of PE-conjugated ferret cross-reactive mouse monoclonal antibody to bovine IFN-γ (clone CC302, AbD-Serotec, Denmark) (Martel et al., Vet. Imuunol. Immunopathol. 132:109-115, 2009). Finally, the cells are analyzed with a flow cytometer. Gating for lymphocyte populations is done as previously described (Aasted et al., Vet. Immunol. Immunopathol. 119:27-37, 2007).

Finally, hemagglutination inhibition assays are performed according to the standard WHO protocol WHO/CDS/CSR/NCS 2002.5 Rev.1 (WHO, 2002, WHO Manual on Animal Influenza Diagnosis and Surveillance, In: Response, CDSam editor). Hemagglutination is measured by the viral agglutination of 0.4% (vol/vol) guinea pig red blood cells (Statens Serum Institut, Denmark) Serum samples are incubated overnight at 37° C. with 4 parts receptor destroying enzyme (RDE) to destroy nonspecific inhibitors of hemagglutination. The reaction is stopped by denaturing the enzyme at 56° C. for 30 minutes. RDE-treated sera is two-fold serially diluted in 96-well v-bottomed microtiter plates (Nunc, Roskilde, Denmark), and an equal volume of virus adjusted to 8 hemagglutination units is added.

Collectively, the data described in the Examples show that multiple oligoribonucleotide sequences derived from the 3′-termini of positive-sense single-stranded RNA viruses or from the 5′-terminus of a negative-sense single RNA virus are capable of activating TLR7 and TLR8 in mouse and human cells, and can induce immune cell activation and strong cytokine production from mouse and human cell cultures. Thus, these nucleic acids can be used for TLR7/8-driven immune stimulation.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. An composition comprising an isolated nucleic acid molecule 10 to 200 nucleotides in length comprising:
(i) a 10 to 40 nucleotide sequence from the first 80 bases from a 5′- or 3′-terminus of a positive-sense single-stranded RNA virus genome; or
(ii) a 10 to 40 nucleotide sequence from the first 80 bases from a 5′-terminus of a negative-sense single-stranded RNA virus genome.

2. The composition of claim 1, wherein the isolated nucleic acid molecule comprises the sequence of any one of SEQ ID NOs: 1-19.

3. The composition of claim 1, wherein the nucleic acid molecule is 10 to 100 nucleotides in length.

4. The composition of claim 1, wherein the nucleic acid molecule is at least partially double-stranded.

5. The composition of claim 1, wherein the nucleic acid molecule has a stabilized backbone.

6. The composition of claim 5, wherein the stabilized backbone:
comprises at least one phosphorothioate internucleoside linkage or is a phosphorothioate backbone; or
comprises at least one pyrophosphate internucleoside linkage or is a pyrophosphate backbone.

7. The composition of claim 1, wherein the nucleic acid molecule comprises at least one deoxyribonucleotide.

8. The composition of claim 1, wherein the nucleic acid molecule is a ribonucleic acid (RNA).

9. The composition of claim 1, wherein the nucleic acid molecule is a Toll-like receptor (TLR) agonist.

10. The composition of claim 9, wherein the TLR agonist is an agonist of TLR8 or TLR7.

11. The composition of claim 1, wherein the positive-sense single-stranded RNA virus is a member of the family Flaviviridae or the family Togaviridae.

12. The composition of claim 11, wherein the positive-sense single-stranded RNA virus is a flavivirus or Chikungunya virus.

13. The composition of claim 12, wherein the flavivirus is a Japanese encephalitis or Murray Valley encephalitis virus.

14. The composition of claim 1, wherein the negative-sense single-stranded RNA virus is a member of the family Filoviridae.

15. The composition of claim 14, wherein the negative-sense single-stranded RNA virus is an Ebola virus.

16. The composition of claim 1, wherein the composition further comprises a condensing agent.

17. The composition of claim 16, wherein the condensing agent is a cationic lipid.

18. The composition of claim 1, further comprising an antigen and/or a carrier.

19. The composition of claim 18, wherein the carrier is a synthetic nanocarrier.

20. The composition of claim 19, wherein the synthetic nanocarrier comprises a biodegradable polymer.

21. The composition of claim 19, wherein the nucleic acid molecule is coupled, covalently or noncovalently, to the surface of the synthetic nanocarrier.

22. The composition of claim 19, wherein the nucleic acid molecule is encapsulated within the synthetic nanocarrier.

23. The composition of claim 19, wherein the synthetic nanocarrier comprises an antigen.

24. An immunostimulatory method, the method comprising
obtaining a composition of claim 1; and
contacting an immune cell with the composition in an amount effective to immuno stimulate the cell.

25. The method of claim 24, wherein the immunostimulated immune cell expresses a type 1 interferon, interferon-γ, tumor necrosis factor α, interleukin-6, interleukin-12, interleukin-10, or interleukin-23.

26. A method for stimulating toll-like receptor (TLR) signaling, the method comprising contacting a cell expressing a TLR with a composition of claim 1 in an amount effective to stimulate signaling by the TLR.

27. The method of claim 26, wherein the TLR is TLR8 or TLR7.

28. A method for stimulating an immune response in a subject, the method comprising administering to a subject a composition of claim 1 in an amount effective to stimulate an immune response in the subject.

29. A method for stimulating an antigen-specific immune response in a subject, the method comprising administering to a subject a composition of claim 1 comprising an antigen in an amount effective to stimulate an antigen-specific immune response in the subject.

30. The method of claim 29, wherein the antigen comprises an allergen, a viral antigen, a bacterial antigen, a hapten, or an antigen that is autologous to the subject or allogeneic to the subject.

31. A method for screening for an antagonist of a toll-like receptor (TLR), the method comprising
contacting a reference cell expressing a TLR with an amount of a composition of claim 1, in the absence of a candidate antagonist of the TLR, and measuring a reference amount of signaling by the TLR;
contacting a test cell expressing the TLR with an amount of the composition, in presence of the candidate antagonist of the TLR, and measuring a test amount of signaling by the TLR; and
identifying the candidate antagonist of the TLR as an antagonist of the TLR when the reference amount of signaling exceeds the test amount of signaling.

32. A method of administering a composition of claim 1 to a subject.

33. The method of claim 32, wherein the subject has a disease or disorder.

34. The method of claim 33, wherein the disease or disorder is selected from the group consisting of: a cancer, an infectious disease, a metabolic disease, a degenerative disease, an autoimmune disease, an inflammatory disease, or an immunological disease.
